(19) United States
(12) Patent Application Publication
(10) Pub. No.: US 2022/0218700 A1
(43) Pub. Date: Jul. 14, 2022

(54) COMBINATION OF AN ?2-ADRENOCEPTOR SUBTYPE C (ALPHA-2C) ANTAGONISTS WITH A TASK1/3 CHANNEL BLOCKER FOR THE TREATMENT OF SLEEP APNEA

(71) Applicant: Bayer Aktiengesellschaft, Leverkusen (DE)

(72) Inventors: Martina DELBECK, Berlin (DE); Michael HAHN, Langenfeld (DE)

(73) Assignee: Bayer Aktiengesellschaft, Leverkusen (DE)

(21) Appl. No.: 17/595,136

(22) PCT Filed: May 4, 2020
(86) PCT No.: PCT/EP2020/062262

§ 371 Date: Nov. 9, 2021

(30) Foreign Application Priority Data
May 9, 2019 (EP) 19173592.7

(51) Int. Cl. A61K 31/496; A61K 9/00; A61P 11/02

(52) U.S. Cl. CPC A61K 31/496; A61P 11/02; A61K 9/0043

(57) ABSTRACT

The present invention relates to a combination of selective blockers of TASK-1 and TASK-3 channels, in particular diazabicyclically substituted imidazo[1,2-a]pyrimidine derivatives and α2-Adrenoceptor subtype C (alpha-2C) antagonists, in particular aryl piperazines of formula (I) for the treatment and/or prophylaxis of sleep-related breathing disorders, preferably obstructive and central sleep apneas and snoring.

The present invention relates to a combination of selective blockers of TASK-1 and TASK-3 channels, in particular diazabicyclically substituted imidazo[1,2-a]pyrimidine derivatives and α2-Adrenoceptor subtype C (alpha-2C) antagonists, in particular aryl piperazines of formula (1) for the treatment and/or prophylaxis of sleep-related breathing disorders, preferably obstructive and central sleep apneas and snoring.

### BACKGROUND OF THE INVENTION

Obstructive sleep apnoea (OSA) is a sleep-related respiratory disorder which is characterized by repetitive episodes of obstruction of the upper airways. When breathing in, the patency of the upper airways is ensured by the interaction of two opposite forces. The dilative effects of the musculature of the upper airways counteract the negative intraluminal pressure, which constricts the lumen. The active contraction of the diaphragm and the other auxiliary respiratory muscles generates a negative pressure in the airways, thus constituting the driving force for breathing. The stability of the upper respiratory tract is substantially determined by the coordination and contraction property of the dilating muscles of the upper airways.

Upper airway collapse in OSA is thought to occur at sleep onset because of the reduction of activity of several upper airway dilator muscles, which as a consequence are unable to maintain the anatomically vulnerable airway open. However, some upper airway dilator muscles, including the genioglossus muscle, which is the most important of the dilating muscles of the upper respiratory airway and which is innervated by the hypoglossal nerve, can increase activity during sleep in response to respiratory stimuli, potentially counteracting some of these changes at sleep onset. It was observed that OSA patients have apnea free intervals in which the genioglossus muscle activity is only 25-40% higher compared with sleep phases with frequent obstructive apneas (Jordan A S, White D P, Lo Y L et al., Airway dilator muscle activity and lung volume during stable breathing in obstructive sleep apnea. Sleep 2009, 32(3): 361-8). Noradrenaline is one of the most potent neuromodulators of hypoglossal motoneuron activity (Horner R. L. Neuromodulation of hypoglossal motoneurons during sleep. Respir Physiol Neurobiol 2008, 164 (1-2): 179-196). It is thought, that decreased noradrenergic drive leads to sleep-dependent decline of hypoglossal motoneuron excitability resulting in reduced upper airway dilator muscle activity, especially reduced genioglossus muscle activity.

Alpha2C adrenoceptors regulate the release of noradrenaline from central noradrenergic neurons, they are autoreceptors involved in presynaptic feedback inhibition of noradrenaline (Hein L. et al, Two functionally distinct alpha2-adrenergic receptors regulate sympathetic neurotransmission Nature 1999, 402(6758): 181-184). An increase in the activity of the motoneurons of the hypoglossal nerve through Alpha2c adrenoceptor antagonism can stabilize the upper airways and protect them from collapse and occlusion. Moreover, also snoring can be inhibited through the mechanism of stabilization of the upper respiratory airways.

For simple snoring, there is no obstruction of the upper airways. By the narrowing of the upper airways, the flow velocity of the inhaled and exhaled air increases. This together with the relaxed muscles causes fluttering of the soft tissues of the mouth and throat in the airflow. This slight vibration generated the typical snoring sounds.

Obstructive snoring (upper airway resistance syndrome, heavy snoring, hypopnea syndrome) is caused by a recurrent partial obstruction of the upper airway during sleep. This results in an increase in airway resistance and thus to an increase in work of breathing with significant intrathoracic pressure fluctuations. The negative intrathoracic pressure development during inspiration can thereby reach values as they occur as a result of a complete airway obstruction in OSA. The pathophysiological effects on the heart, circulation and sleep quality are the same as in obstructive sleep apnea. The pathogenesis is likely the same as in OSA. Obstructive snoring often provides the precursor for OSA (Hollandt J. H. et al., Upper airway resistance syndrome (UARS)-obstructive snoring. HNO 2000, 48(8): 628-634).

Central sleep apnea (CSA) occurs as a result of disturbed brain function or impaired respiratory regulation. CSA is characterized by a lack of drive to breathe during sleep, resulting in repetitive periods of insufficient or absent ventilation and compromised gas exchange. There are several manifestations of CSA. These include high altitude-induced periodic breathing, idiopathic CSA (ICSA), narcotic-induced central apnea, obesity hypoventilation syndrome (OHS), and Cheyne-Stokes breathing (CSB). While the precise precipitating mechanisms involved in the various types of CSA may vary considerably, unstable ventilatory drive during sleep is a principal underlying feature (Eckert D. J. et al., Central sleep apnea: Pathophysiology and treatment. Chest 2007, 131(2): 595-607).

US 2018/0235934 A1 describes methods for treating disorders such as obstructive sleep apnea using agents for promoting hypoglossal motoneuron excitability. As agents for promoting hypoglossal motoneuron excitability a disinhibtor and/or stimulant of central noradrenic neurons is described. In some embodiments the disinhibitor of central noradrenergic neurons is an alpha2-adrenoceptor antagonist such as yohimbine or an alpha2-adrenoceptor subtype A (alpha-2A) antagonists or alpha2-adrenoceptor subtype C (alpha-2C) antagonist. The alpha2-adrenoceptor antagonist are selected from the group consisting of Atipamezole. MK-912, RS-79948, RX 821002, [3H]2-methoxy-idazoxan and JP-1302.

Alpha2C adrenoceptors belong to the family of G-protein coupled receptors. Beside the different Alpha 1-adrenoceptors three different Alpha2-adrenoceptor subtypes exist (Alpha2A, Alpha2B and Alpha2C). They are involved in the mediation of several diverse physiologic effects in different tissues upon stimulation by endogeneous catecholamines (epinephrine, norepinephrine), either derived from synapses or via the blood. Alpha2 adrenoceptors plays an important physiological role, mainly in the cardiovascular system and in the central nervous system. Alpha2A- and Alpha2C-adrenoceptors are the main autoreceptors involved in presynaptic feedback inhibition of noradrenaline in the central nervous system. The potency and affinity of noradrenaline at the Alpha2C-adrenoceptor is higher than that for the Alpha2A-adrenoceptor. The Alpha2C-adrenoceptor inhibits noradrenaline release at low endogenous concentrations of noradrenaline, while Alpha2A -adrenoceptors inhibit noradrenaline release at high endogenous noradrenaline concentrations (Uys M. M. et al. Therapeutic Potential of Selectively Targeting the α2C-Adrenoceptor in Cognition, Depression, and Schizophrenia—New Developments and Future Perspective. Frontiers in Psychiatry 2017, Aug. 14;8:144. doi: 10.3389/fpsyt.2017.00144. eCollection 2017).

A further mechanism to maintain airway patency relies on negative pressure-sensitive nerve endings/mechanoreceptors located in the pharyngeal mucosa. Upon detection of small negative pressures during the respiratory cycle these receptors generate excitatory motor nerve output to the genioglossus muscle via the negative pressure reflex.

The genioglossus muscle plays a decisive role in the pathogenesis of obstructive sleep apnoea. The activity of this muscle increases with decreasing pressure in the pharynx in the sense of a dilative compensation mechanism. Innervated by the Nervus hypoglossus, it drives the tongue forward and downward, thus widening the pharyngeal airway [Verse et al., Somnologie 3, 14-20 (1999)]. Tensioning of the dilating muscles of the upper airways is modulated inter alia via mechanoreceptors/stretch receptors in the nasal cavity/pharynx 1Bouillette et al., J. Appl. Physiol. Respir. Environ. Exerc. Physiol. 46, 772-779 (1979)1. In sleeping patients suffering from serious sleep apnoea, under local anaesthesia of the upper airway an additional reduction of the activity of the genioglossus muscle can be observed [Berry et al., Am. J. Respir. Crit. Care Med. 156, 127-132 (1997)].

In a sleep apnoea model in the anaesthetized pig, intranasal administration of a potassium channel blocker which blocks the TASK-1 channel in the nanomolar range led to inhibition of collapsibility of the pharyngeal respiratory musculature and sensibilization of the negative pressure reflex of the upper airways. It is assumed that intranasal administration of the potassium channel blocker depolarizes mechanoreceptors in the upper airways and, via activation of the negative pressure reflex, leads to increased activity of the musculature of the upper airways, thus stabilizing the upper airways and preventing collapse. By virtue of this stabilization of the upper airways, the TASK channel blockade may be of great importance for obstructive sleep apnoea and also for snoring [Wirth et al., Sleep 36, 699-708 (2013); Kiper et al., Pflugers Arch. 467, 1081-1090 (2015)].

Of particular interest are TASK-1 (KCNK3 or K2P3.1) and TASK-3 (KCNK9 or K2P9.1) of the TASK (TWIK-related acid-sensitive K+channel) subfamily. Functionally, these channels are characterized in that, during maintenance of voltage-independent kinetics, they have “leak” or “background” streams flowing through them, and they respond to numerous physiological and pathological influences by increasing or decreasing their activity. A characteristic feature of TASK channels is the sensitive reaction to a change of the extracellular pH: at acidic pH the channels are inhibited, and at alkaline pH they are activated.

TASK-1 and TASK-3 channels play also a role in respiratory regulation. Both channels are expressed in the respiratory neurons of the respiratory centre in the brain stem, inter alia in neurons which generate the respiratory rhythm (ventral respiratory group with pre-Botzinger complex), and in the noradrenergic Locus caeruleus, and also in serotonergic neurons of the raphe nuclei. Owing to the pH dependency, here the TASK channels have the function of a sensor which translates changes in extracellular pH into corresponding cellular signals [Bayliss et al. Pflugers Arch. 467, 917-929 (2015)]. TASK-1 and TASK-3 are also expressed in the Glomus caroticum, a peripheral chemoreceptor which measures pH, O₂ and CO₂ content of the blood and transmits signals to the respiratory centre in the brain stem to regulate respiration. It was shown that TASK-I knock-out mice have a reduced ventilatory response (increase of respiratory rate and tidal volume) to hypoxia and normoxic hypercapnia [Trapp et al., J. Neurosci. 28, 8844-885(1 (2008)]. Furthermore, TASK-1 and TASK-3 channels were demonstrated in motoneurons of the Nervus hypoglossus. the XIIth cranial nerve, which has an important role in keeping the upper airways open [Berg et al., J. Neurosci. 24_{—} 6693-6702 (2004)].

Aryl piperazines as α2-Adrenoceptor subtype C (alpha-2C) antagonists as well as their preparation and the use thereof as a medicament arc known from WO 2010/058060 A1 where the compounds are disclosed as useful for the treatment for disorders such as disorder propagated by stress, Parkinson's disease, depression, schizophrenia, attention deficit hyperactivity disorder, post-traumatic stress disorder, obsessive compulsive disorder, Tourette's syndrome, blepharospasm or other focal dystonias, temporal lobe epilepsy with psychosis, a drug-induced psychosis, Huntington's disease, a disorder caused by fluctuation of the levels of sex hormones, panic disorder, Alzheimer's disease or mild cognitive impairment. There is nothing disclosed about the use of these compounds in the treatment of sleep-related breathing disorders, preferably obstructive and central sleep apneas and snoring.

The current gold standard treatment for patients with OSA is continuous positive airway pressure

(CPAP). The positive airflow pressure that is generated by an airflow turbine pump splints open the upper airway, reversing all potential causes of pharyngeal collapse, thereby preventing hypopneas, apneas and sleep fragmentation. Unfortunately, up to 50% of all patients with OSA do not tolerate CPAP in the long-term (M. Kohler, D. Smith, V. Tippett et al., Thorax 2010 65(9):829-32: Predictors of long-term compliance with continuous positive airway pressure). Therefore, there is still the need to find effective therapeutic agents for the treatment and/or prophalxis of sleep-related breathing disorders such as obstructive sleep apnea. Therefore the object of the present invention is to provide an effective therapeutic agent for the treatment and/or prophalxis of sleep-related breathing disorders, for example of obstructive sleep apnea. central sleep apnea and snoring.

Surprisingly, it has now been found that the combination of an a2-Adrenoceptor subtype C (alpha-2C) antagonists with a TASK1/3 channel blocker inhibit upper airway collapsibility with improved efficacy compared to each treatment alone and is thus suitable for the production of medicaments for the use in the treatment and/or prophylaxis of sleep-related breathing disorders, preferably obstructive and central sleep apneas and snoring. It was found that a synergism of the combination of an a2-Adrenoceptor subtype C (alpha-2C) antagonists with a TASK1/3 channel blocker allows lower doses of each treatment compared to each treatment alone.

The present invention relates to combinations of compounds of formula (I)

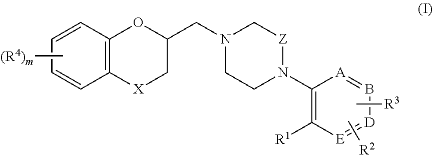

wherein
X is O, S or CH₂,
Z is —[CH₂]ₙ—.
A, B, D and E are independently C or N, provided that at least three of A, B, D and E are C.
R¹ is H, halogen, hydroxy, (C₁-C₆)alkyl, (C₁-C₆)alkoxy, hydroxy(C₁-C₆)alkyl, (C₁-C₆)alkoxy(C₁-C₆)alkyl, halo(C₁-C₆)alkoxy, halo(C₁-C₆)alkoxy(C₁-C₆)alkyl, hydroxy(C₁-C₆)alkoxy(C₁-C₆)alkyl, (C₁-C₆)alkoxy(C₁-C₆)alkoxy(C₁-C₆)alkyl, (C₁-C₆)alkoxy-(C═O)—, CN, (R₅)₂N—, (R₅)₂N—(C₁-C₆)alkyl, (R₅)₂N—(C═O)—, SH—(C₁-C₆)alkyl, hydroxy(C₁-C₆)alkyl-S—(C₁-C₆)alkyl, (C₁-C₆)alkoxy(C₁-C₆)alkyl-S—(C₁-C₆)alkyl, hydroxy(C₁-C₆)alkyl-S(Op)-(C₁-C₆)alkyl, (C₁-C₆)alkoxy(C₁-C₆)alkyl-S(Op)-(C₁-C₆)alkyl or furyl;
R² is H, halogen, (C₁-C₆)alkyl, (C₁-C₆)alkoxy or hydroxy(C₁-C₆)alkyl;
R³ is H, halogen, (C₁-C₆)alkyl or phenyl.
R⁴ is halogen, hydroxy, (C₁-C₆)alkyl, (C₁-C₆)alkoxy, CN or (R⁵)₂N—;
R⁵ is, independently at each occurence, H, (C₁-C₆)alkyl or (C₁-C₆)alkoxy(C₁-C₆)alkyl;
m is 0, 1 or 2;
n is 1 or 2; and
p is 1, or 2,
with compounds of the formula (II)

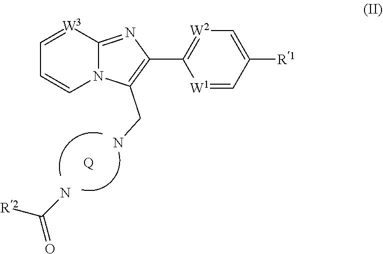

in which
the ring Q represents a piperazine or a diazaheterobicyclic system of the formula

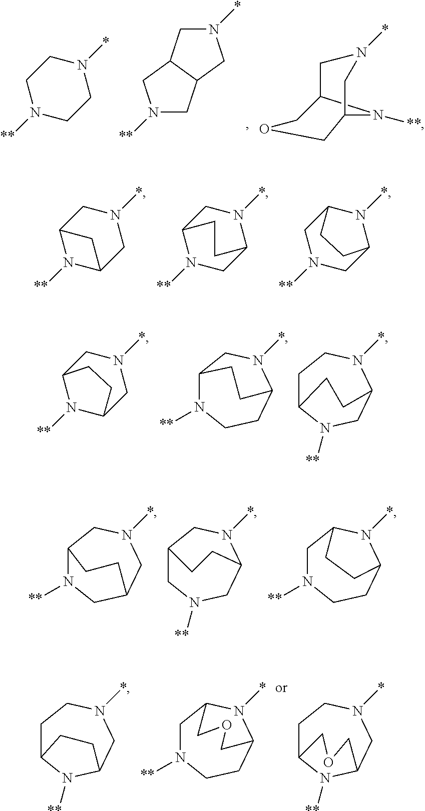

in which * denotes the bond to the adjacent CHR′² group and ** the bond to the carbonyl group,
W¹, W² or W³ represents CH or N,
R′¹ represents halogen, cyano, (C₁-C₄)-alkyl, cyclopropyl or cyclobutyl
where (C₁-C₄)-alkyl may be up to trisubstituted by fluorine and cyclopropyl and cyclobutyl may be up to disubstituted by fluorine,
and
R′² represents (C₄-C₆)-cycloalkyl in which a ring CH₂ group may be replaced by —O—,
or
R′² represents a phenyl group of the formula (a), a pyridyl group of the formula (b) or (c) or an azole group of the formula (d), (e), (f) or (g),

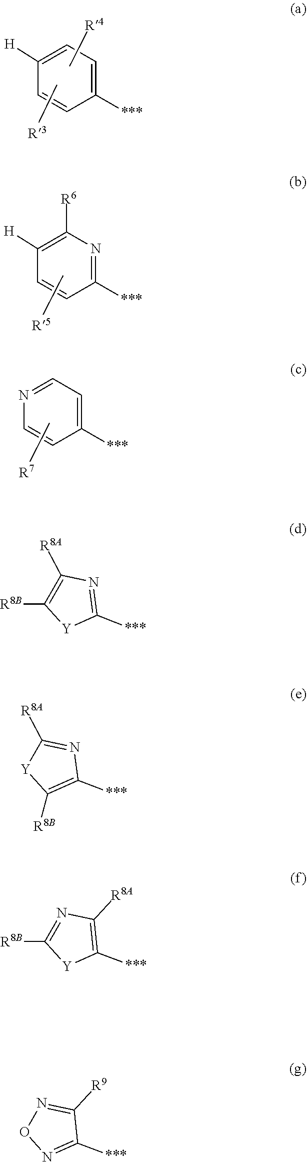

in which *** marks the bond to the adjacent carbonyl group and
R′³ represents hydrogen, fluorine, chlorine, bromine or methyl,
R′⁴ represents hydrogen, fluorine, chlorine, bromine, cyano, (C₁-C₃)-alkyl or (C₁-C₃)-alkoxy, where (C₁-C₃)-alkyl and (C₁-C₃)-alkoxy may each be up to trisubstituted by fluorine.
R′⁵ represents hydrogen, fluorine, chlorine, bromine or methyl,
R⁶ represents hydrogen, (C₁-C₃)-alkoxy, cyclobutyloxy, oxetan-3-yloxy, tetrahydrofuran-3-yloxy, tetrahydro-2H-pyran-4-yloxy, mono-(C₁-C₃)-alkylamino, di-(C₁-C₃)-alkylamino or (C₁-C₃)-alkylsulfanyl,
where (C₁-C₃)-alkoxy may be up to trisubstituted by fluorine,
R⁷ represents hydrogen, fluorine, chlorine, bromine, (C₁-C₃)-alkyl or (C₁-C₃)-alkoxy,
R^{8A} and R^{8B} are identical or different and independently of one another represent hydrogen, fluorine, chlorine, bromine, (C₁-C₃)-alkyl, cyclopropyl or (C₁-C₃)-alkoxy
where (C₁-C₃)-alkyl and (C₁-C₃)-alkoxy may each be up to trisubstituted by fluorine,
R⁹ represents hydrogen, (C₁-C₃)-alkyl or amino
and
wherein in subformula (d)
   Y represents O, S or N(CH₃),
wherein in subformula (e) and (f)
   Y represents O or S.
or
R′² represents an —OR¹⁰ or —NR¹¹R¹² group in which
   R¹⁰ represents (C₁-C₆)-alkyl, (C₄-C₆)-cycloalkyl or [(C₃-C₆)-cycloalkyl]methyl,
   R¹¹ represents hydrogen or (C₁-C₃)-alkyl
   and
   R¹² represents (C₁-C₆)-alkyl, (C₃-C₆)-cycloalkyl, phenyl or benzyl, 1-phenylethyl or 2-phenylethyl,
      where (C₁-C₆)-alkyl may be up to trisubstituted by fluorine,
      and
      where phenyl and the phenyl group in benzyl, 1-phenylethyl and 2-phenylethyl may be up to trisubstituted by identical or different radicals selected from the group consisting of fluorine, chlorine, methyl, ethyl, trifluoromethyl, methoxy, ethoxy, trifluoromethoxy and (trifluoromethyl)sulfanyl,
   or
   R¹¹ and R¹² are attached to one another and, together with the nitrogen atom to which they are bonded, form a pyrrolidine, piperidine, morpholine or thiomorpholine ring, or

R¹¹ and R¹² are attached to one another and, together with the nitrogen atom to which they are bonded, form a tetrahydroquinoline ring of the formula (c) or a tetrahydroisoquinoline ring of the formula (d),

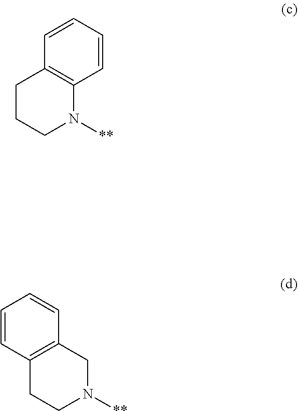

in which ** marks the bond to the carbonyl group,
and the salts, solvates and solvates of the salts thereof.

In a further embodiment of the present invention relates to combinations of compounds of formula (I)
wherein
X is O;
Z is —[CH₂]ₙ—,
A is N;
B, D and E are C;
R¹ is halogen, (C₁-C₆)alkyl, (C₁-C₆)alkoxy, hydroxy(C₁-C₆)alkyl, (C₁-C₆)alkoxy(C₁-C₆)alkyl, halo(C₁-C₆)alkoxy, halo(C₁-C₆)alkoxy(C₁-C₆)alkyl, (C₁-C₆)alkoxy-(C═O)—, CN, (R₅)₂N-(C₁-C₆)alkyl, (R₅)₂N—(C═O)— or furyl;
R² is H, halogen, (C₁-C₆)alkyl or hydroxy(C₁-C₆)alkyl;
R³ is H, (C₁-C₆)alkyl or phenyl;
R⁵ is, independently at each occurence, H or (C₁-C₆)alkyl;
m is 0; and
n is 1
and compounds of formula (III)
wherein
the ring Q represents a piperazine or a diazaheterobicyclic system of the formula

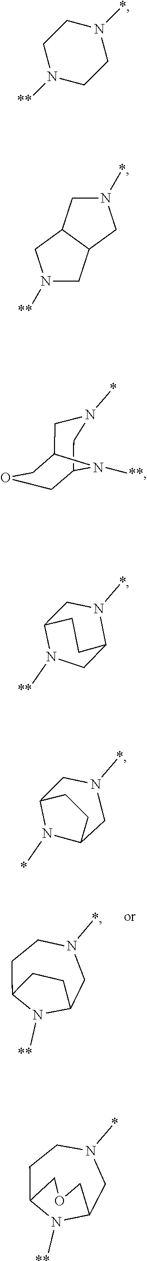

in which * denotes the bond to the adjacent CHR² group and ** the bond to the carbonyl group,
W² represents CH,
W¹, W³ represents CH or N,
R′¹ represents fluorine, chlorine, bromine, methyl, tert.-butyl, isopropyl, cyclopropyl or cyclobutyl,
and
R′² represents cyclobutyl, cyclopentyl or cyclohexyl,
or
R′² represents a phenyl group of the formula (a), a pyridyl group of the formula (b) or an azole group of the formula (d) or formula (g)

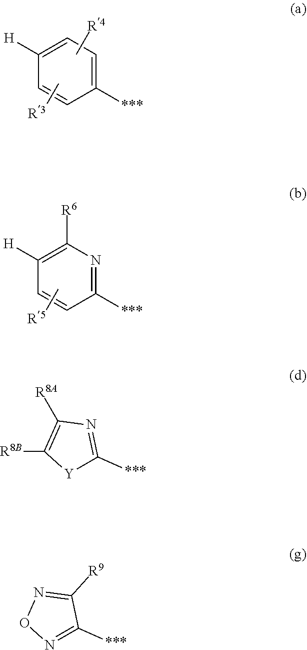

in which *** marks the bond to the adjacent carbonyl group and
R′³ represents hydrogen, fluorine or chlorine,
R′⁴ represents fluorine, chlorine, methyl, isopropyl, methoxy or ethoxy,
R′⁵ represents hydrogen, fluorine, chlorine, bromine or methyl,
R⁶ represents methoxy, difluoromethoxy, trifluoromethoxy, isopropoxy, cyclobutyloxy or methylsulfanyl,
R^{8A} and R^{8B} are identical or different and independently of one another represent hydrogen, methyl, trifluoromethyl, ethyl, isopropyl or cyclopropyl,
and
R⁹ represents methyl or amino
Y represents O or S or N(CH₃)
and the salts, solvates and solvates of the salts thereof.

In a preferred embodiment of the present invention is directed to combinations of compounds of formula (I) which are selected from the group consisting of:

methyl 2-(4-((2,3-dihydrobenzo [b][1,4]dioxin-2-yl)methyl)piperazin-1-yl)benzoate, (2-(4-((2,3-dihydrobenzo[b][1,4]dioxin-2-yl)methyl)piperazin-1-yl)phenyl)methanol,1-((2,3-dihydrobenzo[1)][1,4]dioxin-2-yl)methyl)-4-(2-(methoxymethyl)phenyl)piperazine, 2-(4-((2,3-dihydrobenzo[b] [1,4]dioxin-2-yl)methyl)piperazin-1-yl)benzonitrile, (2-(4-((2,3-dihydrobenzo[b][1,4]dioxin-2-yl)methyl)piperazin-1-yl)phenyl)methanamine, 1-(2-(4-((2,3-dihydrobenzo[b][14]dioxin-2-yl)methyl)piperazin-1-yl)phenyl)-N-methylmethanamine, 1-((2,3-dihydrobenzo[b][1,4]dioxin-2-yl)methyl)-4-(2-(ethoxymethyl)phenyl)piperazine, 2-(2-(4-((2,3-dihydrobenzo[b][1,4]dioxin-2-yl)methyl)piperazin-1-yl)phenyl)propan-2-ol, 1-((2,3-dihydrobenzo[b][1,4]dioxin-2-yl)methyl)-4-(3-(methoxymethyl)pyridin-2-yl)piperazine, (S)-(2-(4-((7-fluoro-2,3-dihydrobenzo[b][1,4]dioxin-2-yl)methyl)-piperazin-1-yl)pyridin-3-yl)methanol, (5)-(2-(4-((7-fluoro-2,3-dihydrobenzo[b][1,4]dioxin-2-yl)methyl)-piperazin-1-yl)pyridin-3-yl)methanol·HCl, (S)-1-((7-fluoro-2,3-dihydrobenzo[b][1,4]dioxin-2-yl)methyl)-4-(3-(methoxymethyl)pyridin-2-yl)piperazine·HCl, (S)-1-((2,3-dihydrobenzo[b][1,4]dioxin-2- yl)methyl)-4-(3-((2-fluoroethoxy)methyl)pyridin-2-yl)piperazine, 1-(2,3-dichlorophenyl)-4-((2,3-dihydrobenzo[b][1,4]dioxin-2-yl)methyl)piperazine, (2-(4-((2,3-dihydrobenzo[b][1,4]dioxin-2-yl)methyl)piperazin-1-yl)pyridin-3-yl)methanol, (S)-(2-(4-((2,3- dihydrobenzo[b][1,4]dioxin-2-yl)methyl)piperazin-1-yl)pyridin-3-yl)methanol, (S)-1((2,3-dihydrobenzo[b][1,4]dioxin-2-yl)methyl)-4-(2-(methoxymethyl)phenyl)piperazine, (R)-1-((2,3-dihydrobenzo [b][1,4]dioxin-2-yl)methyl)-4-(2-(methoxymethyl)phenyl)piperazine, (S)-(2-(4-((2,3- dihydrobenzo[b][1,4]dioxin-2-yl)methyl)piperazin-1-yl)phenyl)methanol, (S)-1-((2,3-dihydrobenzo[b][1,4]dioxin-2-yl)methyl)-4-(3-(methoxymethyl)pyridin-2-yl)piperazine (1-((2,3 -dihydrobenzo[b][1,4 ]oxathiin-2-yl)methyl)-4-(2-(methoxymethyl)phenyl)piperazine, 1-(chroman-2-yl)methyl)-4-(2-(methoxymethyl)phenyl)piperazine, (2-(4-((2,3-dihydrobenzo[b ][1,4]dioxin-2-yl)methyl)piperazin-1-yl)-6-fluorophenyl)methanol, (2-(4-((2,3-dihydrobenzo[b ][1,4]dioxin-2-yl)methyl)piperazin-1-yl)-3-fluorophenyl)methanol, (2-(4-((2,3-dihydrobenzo[b][1,4]dioxin-2-yl)methyl)piperazin-1-yl)-5-fluorophenyl)methanol, (S)-1-((2,3-dihydrobenzo[b][1,4]dioxin-2-yl)methyl)-4-(2-propylphenyl)piperazine, (S)-1-((2,3-dihydrobenzo[b][1,4]dioxin-2-yl)methyl)-4-(2-(trifluoromethoxy)phenyl)piperazine, (S)-1-(biphenyl-3-yl)-4-((2,3-dihydrobenzo[b][1,4]dioxin-2-yl)methyl)piperazine, (S)-1-((2,3-dihydrobenzo[b][1,4]dioxin-2 -yl)methyl)-4-(2-(furan-2-yl)phenyl)piperazine, (S)-ethyl-2-(4-((2,3-dihydrobenzo[b][1,4]dioxin-2-yl)methyl)piperazin-1-yl)benzoate, (S)-1-((2,3- dihydrobenzo[b][1,4]dioxin-2-yl)methyl)-4-o-tolylpiperazine, (S)-1-((2,3-dihydrobenzo[b][1,4]dioxin-2-yl)methyl)-4-m-tolylpiperazine, (S)-(3-(4-((2,3-dihydrobenzo[b][1,4]dioxin-2-yl)methyl)piperazin-1-yl)-4-methylphenyl)methanol, (S)-(3-(4-((2,3-dihydrobenzo[b][1,4]dioxin-2-yl)methyl)piperazin-1-yl)phenyl)methanol, (S)-2-(2-(4-((2,3-dihydrobenzo[b][1,4]dioxin-2-yl)methyl)piperazin-1-yl)phenyl)ethanol, methyl 2-(4-((2,3-dihydrobenzo[b][1,4]dioxin-2-yl)methyl)-1,4-diazepan-1-yl)benzoate, (2(4-((2,3-dihydrobenzo[b][1,4]dioxin-2-yl)methyl)-1,4-diazepan-1-yl)phenyl)methanol, 2-(4-((2,3-dihydrobenzo[b][1,4]dioxin-2-methyl)-1,4-diazepan-1-yl)nicotinonitrile, 2-(4-(2,3-dihydrobenzo[b][1,4]dioxin-2-yl)methyl)-1,4-diazepan-1-yl)nicotinamide, (2-(4-((2,3-dihydrobenzo[b][1,4]dioxin-2-yl)methyl)-1,4-diazepan-1-yl)pyridin-3-yl)methanol or (S)-(2-(4-((2,3-dihydrobenzo[b][1,4]dioxin-2-yl)methyl)-1,4-diazepan-1-yl)pyridin-3-yl)methanol

and compounds of formula (H) which are selected from the group consisting of:

(4-{[2-(4-Bromophenyl)imidazo[1,2-a]pyridin-3-yl]methyl}piperazin-1-yl)(cyclopentyl)methanone, (4-{[2-(4-Chlorophenyl)imidazo[1,2-a]pypiperazin-1-yl](cyclopentyl)methanone, (4-{[2 -(4-Chlorophenyl)imidazo[1,2-a]pyridin-3 -yl]methyl}piperazin-1-yl)(6-methoxypyridin-2-yl) methanone, (4 -{[2-(4-Bromophenyl)imidazo[1,2 -a]pyridin-3-yl]methyl}piperazin-1-yl)(2-fluorophenyl)methanone, (4-{[1 -(4-Bromophenyl)imidazo[1,2a]pyridin-3-yl]methyl}piperazin-1-yl)(3-methoxyphenyl)methanone, (4-{[1-(4-Bromophenyl)imidazo[1,2-a]pyridin-3-yl]methyl}piperazin-1-yl)(2-chloro-5-fluorophenyl)methanone, (4-{[1(4-Chlorophenyl)imidazo[1,2-a]pyridin-3-yl]methyl}piperazin-1-yl)(2-fluorophenyl)methanone, (4-{[2-(4-Fluorophenyl)imidazo[1,2-a]pyridin-3-yl]methyl}piperazin-1-yl)(cyclohexyl)methanone, (4-{[1-(4-bromophenyl)imidazo[1,2-a]pyridin-3-yl]methyl}piperazin-1-yl)(cyclohexyl)methanone, (4-{[1-(4-bromophenyl)imidazo[1,2-a]pyridin-3-yl]methyl}piperazin-1-yl)(tetrahydrofuran-3-yl)methanone, (4-{[1-(4-bromophenyl)imidazo[1,2-a]pyridin-3-yl]methyl}piperazin-1-yl)(cyclobutyl)methanone, (4-{[1-(4-bromophenyl)imidazo[1,2-a]pyridin-3-yl]methyl}piperazin-1-yl)(2-methoxyphenyl)methanone, (4-{[1-(4-bromophenyl)imidazo[1,2-a]pyridin-3-yl]methyl}piperazin-1-yl)(5-fluoro-2-methoxyphenyl)methanone, (4-{[1-(4-bromophenyl)imidazo[1,2-a]pyridin-3-yl]-methyl}piperazin-1-yl)(2 -methylphenyl)methanone, (4-{[1-(4-bromophenyl)imidazo[1,2-a]pyridin-3-yl]-methyl}piperazin-1-yl)(5-fluoro-2-methylphenyl)methanone, (2-chloro-5-fluorophenyl)(4-{[2-(4-chlorophenyl)imidazo[1,2-a]pyridin-3-yl]methyl}piperazin-1-yl)methanone, (4-{[1-(4-chlorophenyl)imidazo[1,2-a]pyridin-3-yl]methyl}piperazin-1-yl)(cyclohexyl)methanone ((4-{[1-(4-chlorophenyl)imidazo[1,2-a]pyridin-3-yl]methyl}piperazin-1-yl)(cyclobutyl)methanone, (4-{[1-(4-chlorophenyl)imidazo[1,2-a]pyridin-3-yl]methyl}piperazin-1-yl)(3-methoxyphenyl)methanone, (4-{[2-(4-chlorophenyl)imidazo[1,2-a]pyridin-3-yl]methyl}piperazin-1-yl)(2-methoxyphenyl)methanone (4-{[1-(4-chlorophenyl)imidazo[1,2-a]pyridin-3-yl]methyl}piperazin-1-yl)(5-fluoro-2-methoxyphenyl)methanone, (4-{[2-(4-chlorophenyl)imidazo[1,2-a]pyridin-3 -yl]methyl}piperazin-1-yl)(2-methylphenyl)methanone, (4-{[1-(4-chlorophenyl)imidazo[1,2-a]pyridin-3 methyl}piperazin-1-yl)(5-fluoro-2-methylphenyl)methanone, (4-{[2-(4-chlorophenyl)imidazo[1,2-a]pyridin-3-yl]methyl}piperazin-1-yl)[3-(trifluoromethoxy)phenyl]methanone, (4-{[1-(4-chlorophenyl)imidazo[1,2-a]pyridin-3-yl]methyl}piperazin-1-yl)[3-(trifluoromethyl)phenyl]methanone, ((4-{[1-(4-chlorophenyl)imidazo[1,2-a]pyridin-3-yl]methyl}piperazin-1-yl)(pyridin-2 -yl)methanone (4-{[1-(4-chlorophenyl)imidazo[1,2-a]pyridin-3-yl]methyl}piperazin-1-yl)(2-fluoro-5-methoxyphenyl)methanone, (4-{[1-(4-chlorophenyl)imidazo[1,2-a]pyridin-3-yl]methyl}piperazin-1-yl)(2-ethoxyphenyl)methanone, (2-chloro-5-methoxyphenyl)(4-{[1-(4-chlorophenyl)imidazo[1,2-a]pyridin-3-yl]methyl}piperazin-1-yl)methanone, (4-{[2-(4-chlorophenyl)imidazo[1,2a]pyridin-3-yl]methyl}piperazin-1-yl)(tetrahydro-2H-pyran-2-methanone, (4-{[1-(4-chlorophenyl)imidazo[1,2-a]pyridin-3-yl]methyl}piperazin-1-yl)(3-isopropoxyphenyl)methanone, 2-1(4-{[1-(4-chlorophenyl)imidazo[1,2-a]pyridin-3-yl]methyl}piperazin-1-yl)carbonyl]benzonitrile, (4- {[2-(4-chlorophenyl)imidazo[1,2-a]pyridin-3-yl]methyl}piperazin-1-yl)(3-isopropylphenyl)methanone, (4-{[1-(4-chlorophenyl)imidazo[1,2-a]pyridin-3-yl]methyl}piperazin-1-yl)(2-isopropylphenyl)methanone, (4-{[1-(4-chlorophenyl)imidazo[1,2-a]pyridin-3-yl]methyl}piperazin-1-yl)(tetrahydrofuran-2-yl)methanone, (3-chlorophenyl)(4-{[2-(4-chlorophenyl)imidazo[1,2-a]pyridin-3-yl]methyl}piperazin-1-yl)methanone, (2-chlorophenyl)(4-{[1-(4-chlorophenyl)imidazo[1,2-a]pyridin-3-yl]methyl}piperazin-1-yl)methanone, (4-{[1-(4-chlorophenyl)imidazo[1,2-a]pyridin- 3-yl]methyl}piperazin-1-yl)[6-(2,2,2-trifluoroethoxy)pyridin-2-yl]methanone, (4-{[2-(4-chlorophenyl)imidazo[1,2 -a]pyridin-3-yl]methyl}piperazin-1-yl)(6-isopropoxypyridin-2-yl)methanone, (4-{[1-(4-chlorophenyl)imidazo[1,2-a]pyridin-3-yl]methyl}piperazin-1-yl)(6-methoxy-4-methylpyridin-2-yl)methasone, [2-(4-chlorophenyl)imidazo[1,2-a]pyridin-3-yl]methyl}piperazin-1-yl)[6- (cyclobutyloxy)pyridin-2-yl]methanone, (3-bromo-6-methoxypyridin-2-yl)(4-{[1-(4-chlorophenyl)imidazo[1,2-a]pyridin-3-yl]methyl}piperazin-1-yl)methanone, (3-chloro-6-methoxypyridin-2-yl)(4-{[1-(4-chlorophenyl)imidazo[1,2-a]pyridin-3-yl]methyl}piperazin-1-yl)methanone, (4-{[1-(4-chlorophenyl)imidazo[1,2-a]pyridin-3-yl]methyl}piperazin-1-yl)[6-(difluoromethoxy)pyridin-2-yl]methanone, (4-{[1-(4-chlorophenyl)imidazo[1,2-a]pyridin-3-yl]methyl}piperazin-1-yl)(6-ethoxypyridin-2-yl)methanone, (4-{[1-(4-chlorophenyl)imidazo[1,2-a]pyridin-3-yl]methyl}piperazin-1-yl)[6-(tetrahydro-2H-pyran-4-yloxy)pyridin-2-yl]methanone, (4-{[1-(4-bromophenyl)imidazo[1,2-a]pyridin-3-yl]methyl}perazin-1-yl)(6-methoxypyridin-2-yl)methanone, (4-{[1-(4-fluorophenyl)imidazo[1,2-a]pyridin-3-yl]methyl}piperazin-1- yl)(cyclopentyl)methanone, (4-{[2-(4-fluorophenyl)imidazo[1,2-a]pyridin-3-yl]methyl}piperazin-1-yl)(cyclobutyl)methanone, (5-fluoro-2-methoxyphenyl)(4-{[1-(4-fluorophenyl)imidazo[1,2-a]pyridin-3-yl]methyl}piperazin-1-yl)methanone, (2-chloro-5-fluorophenyl)(4-{[1-(4-fluorophenyl)imidazo[1,2-a]pyridin-3-yl]methyl}piperazin-1-yl)methanone, (4-{[1-(4-fluorophenyl)imidazo[1,2-a]pyridin-3-yl]methyl}piperazin-1-yl)(2-methoxyphenyl)methanone, (2-fluorophenyl)(4-{[1-(4-isopropylphenyl)imidazo[1,2-a]pyridin-3-yl]methyl}piperazin-1-yl)methanone, cyclopentyl(4-{[2-(4-isopropylphenyl)imidazo[1,2-a]pyridin-3-yl]methyl}piperazin-1-yl)methanone (4-{[1-(4-isopropylphenyl)imidazo[1,2-a]pyridin-3-yl]methyl}piperazin-1-yl)(6-methoxypyridin-2-yl)methanone, cyclopentyl(4-{[1-(4-methylphenyl)imidazo[1,2-a]pyridin-3-yl]methyl}piperazin-1-yl)methanone, cyclohexyl(4-{[2-(4-methylphenyl)imidazo[1,2-a]pyridin-3-yl]methyl}piperazin-1-yl)methanone, (2- methoxyphenyl)(4-{[2-(4-methylphenyl)imidazo[1,2-a]pyridin-3-yl]methyl}piperazin-1-yl)methanone, (6-methoxypyridin-2-yl)(4-{[2-4-methylphenyl)imidazo[1,2-a]pyridin-3-yl]methyl}piperazin-1-yl)methanone, (4-(3-{[4-(2-fluorobenzoyl)piperazin-1-yl]methyl}imidazo[1,2-a]pyridin-2-yl)benzonitrile, 4-[3-({4-[(6-methoxypyridin-2-yl)carbonyl]piperazin-1yl}methyl)imidazo[1,2 -a]pyridin-2-yl]benzonitrile, 4-(3-{[4-(cyclopentylcarbonyl)piperazin-1-yl]methyl}imidazo[1,2-a]pyridin-2-yl)benzonitrile, 4-(3-{[4-(cyclohexylcarbonyl)piperazin-1-yl]methyl}imidazo[1,2-a]pyridin-2-yl)benzonitrile, (4-{[2-(4-tert-butylphenyl)imidazo[1,2-a]pyridin-3 -yl]methyl}piperazin-1-yl)(6-methoxypyridin-2-yl)methanone, (4-{[2-(4-tert-butylphenyl)imidazo[1,2-a]pyridin-3-yl]methyl}piperazin-1-yl)(2-fluorophenyl)methanone, (4-{[2-(4-tert-butylphenyl)imidazo[1,2-a]pyridin-3-yl]methyl}piperazin-1-yl)(cyclopentyl)methanone, (4-{[2-(4-Chlorophenyl)imidazo[1,2-a]pyridin-3-yl]methyl}piperazin-1-yl)[6-(trifluoromethoxy)pyridin-2-yl]methanone, (4-{[2-(4- Chlorophenyl)imidazo[1,2-a]pyridin-3-yl]methyl}piperazin-1-yl)(3-fluoro-6-methoxypyridin-2-yl)methanone, (4-{[2-(4-Cyclopropylphenyl)imidazo[1,2-a]pyridin-3-yl]methyl}piperazin-1-yl)(2- fluorophenyl)methanone, 4-(3-{[4-(2-fluoro-5-methoxybenzoyl)piperazin-1-yl]methyl}imidazo[1-2-a]pyridin-2-yl)benzo-nitrile, 4-[3-({4-[(6-methoxy-3-methylpyridin-2-yl)carbonyl]piperazin-1-yl]methyl}imidazo[1,2-a]pyridin-2-yl)benzonitrile, (4-{[2-(4-chlorophenyl)imidazo[1,2- a]pyridin-3-yl]-methyl}piperazin-1-yl)(6-methoxy-3-methylpyridin-2-yl)methanone, (4-{[2-(4-tert-butylphenyl)imidazo[1,2-a]-pyridin-3-yl]methyl}piperazin-1-yl)(6-methoxy-3-methylpyridin-2-yl)methanone, (4-{[2-(4-bromophenyl)imidazo[1,2-a]pyridin-3-yl]-methyl}piperazin-1-yl)(6- methoxy-3-methyl-pyridin-2-yl)methanone; tert-butyl 5-{[2-(4-chlorophenyl)imidazo[1,2-a]pyridin-3-yl]methyl}hexahydropyrrolo[3,4-c]pyrrole-2(1H)-carboxylate, tert-butyl 5-{[2-(4-bromophenyl)imidazo[1,2-a]pyridin-3-yl]methyl}hexahydropyrrolo[3,4-c]pyrrole-2(1H)-carboxylate, tert-butyl 5 -{[2-(4-isopropylphenyl)imidazo[1,2-a]pyridin-3-yl]methyl}hexahydropyrrolo[3,4-c]pyrrole-2(1H)-carboxylate, [5-{[2-(4-Chlorophenyl)imidazo[1,2-a]pyridin-3-yl]methyl}hexahydropyrrolo[3,4-c]pyrrol-2(1H)-yl](6-methoxypyridin-2-yl)methanone, [5-{[2-(4-Bromophenyl)imidazo[1,2-a]pyridin-3-yl]methyl}hexahydropyrrolo[3,4-c]pyrrol-2(1H)-yl](cyclopentyl)methanone, [5-{[2-(4-Bromophenyl)imidazo[1,2-a]pyridin-3-yl]methyl}hexahydropyrrolo[3,4- c]pyrrol-2(1H)-yl](2-fluorophenyl)methanone, [5-{[2-(4-Bromophenyl)imidazo[1,2-a]pyridin-3-yl]methyl}hexahydropyrrolo[3,4-c]pyrrol-2(1H)-yl](2-chloro-5-fluorophenyl)methanone, [5-{[2-(4-Bromophenyl)imidazo[1,2-a]pyridin-3-yl]methyl}hexahydropyrrolo[3,4-c]pyrrol-2(1H)-yl](cyclohexyl)methanone, [5-{[2-(4-Bromophenyl)imidazo[1,2-a]pyridin-3-yl]methyl}hexahydropyrrolo[3,4-c]pyrrol-2(1H)-yl](cyclobutyl)methanone, [5-{[2-(4-Bromophenyl)imidazo[1,2-a]pyridin-3-yl]methyl}hexahydropyrrolo[3,4- c]pyrrol-2(1H)-yl](3-methoxyphenyl)methanone, [5-{[2-(4-Bromophenyl)imidazo[1,2-a]pyridin-3-yl]methyl}hexahydropyrrolo[3,4-c]pyrrol-2(1H)-yl](2-methoxyphenyl)methanone, [5-{[2-(4-Bromophenyl)imidazo[1,2-a]pyridin-3-yl]methyl}hexahydropyrrolo[3,4-c]pyrrol-2(1H)-yl](5-fluoro-2-methoxyphenyl)methanone, [5-{[2(4-Bromophenyl)imidazo[1,2-a]pyridin-3-yl]methyl}[3,4-c]pyrrol-2(1H)-yl](2-methylphenyl)methanone, [5-{[2-(4-Chlorophenyl)imidazo[1,2-a]pyridin-3-yl]methyl}hexahydropyrrolo[3,4-c]pyrrol-2(1H)-yl](2-fluorophenyl)methanone, (2-Chloro-5-fluorophenyl)[5-{[2-(4-chlorophenyl)imidazo[1,2-a]pyridin-3-yl]methyl}hexahydropyrrolo[3,4-c]pyrrol-2(1H)-yl]methanone, [5-{[2-(4-Chlorophenyl)imidazo[1,2-a]pyridin-3-yl]methyl}hexahydropyrrolo[3,4-c]pyrrol-2(1H)-yl](cyclohexyl)methanone, [5-{[2-(4-Chlorophenyl)imidazo[1,2-a]pyridin-3-yl]methyl}hexahydropyrrolo[3,4-c]pyrrol-2(1H)-yl](3-methoxyphenyl)methanone, [5-{[2-(4-Chlorophenyl)imidazo[1,2-a]pyridin-3-yl]methyl}hexahydropyrrolo[3,4 -c]pyrrol-2(1H)-yl](2-methoxyphenyl)methanone, [5-{[2-(4-Chlorophenyl)imidazo[1,2-a]pyridin-3-yl]methyl}hexahydropyrrolo[3,4-c]pyrrol-2(1H)-yl](5-fluoro-2-methoxyphenyl)methanone, [5-{[2-(4-Chlorophenyl)imidazo[1,2-a]pyridin-3-yl]methyl}hexahydropyrrolo[3,4 -c]pyrrol-2(1H)-yl](2-methylphenyl)methanone, [5-{[2-(4-Chlorophenyl)imidazo[1,2-a]pyridin-3-yl]methyl}hexahydropyrrolo[3,4-c]pyrrol-2(1H)-yl](5-fluoro-2-methylphenyl)methanone, [5-{[2-(4-Chlorophenyl)imidazo[1,2-a]pyridin-3-yl]methyl}hexahydropyrrolo[3,4-c]pyrrol-2([3-(trifluoromethoxy)phenyl]methanone, [5-{[2-(4-Chlorophenyl)imidazo[1,2-a]pyridin-3 hexahydropyrrolo[3,4-c]pyrrol-2(1H)-yl][3-(trifluoromethyl)phenyl]methanone, [5-{[2-(4-Bromophenyl)imidazo[1,2-a]pyridin-3-yl]methyl}hexahydropyrrolo[3,4-c]pyrrol-2(1H)-yl](6-methoxypyridin-2-yl)methanone, [5-{[2-(4-Isopropylphenyl)imidazo[1,2-a]pyridin-3-yl]methyl}hexahydropyrrolo[3,4-c]pyrrol-2 (1H)-yl](6-methoxypyridin-2-yl)methanone, (2-Fluorophenyl)[5-{[2-(4-isopropylphenyl)imidazo[1,2a]pyridin-3-yl]methyl}hexahydropyrrolo[3,4-c]pyrrol-2(1H)-yl]methanone, [5-{[2-(4-Isopropylphenyl)imidazo[1,2-a]pyridin-3-yl]methyl}hexahydropyrrolo [3,4-c]pyrrol-2(1H)-yl](3-methoxyphenyl)methanone, Cyclopentyl[5-{8 2-(4-isopropylphenyl)imidazo[1,2-a]pyridin-3-yl]methyl}hexahydropyrrolo [3,4-c]pyrrol-2(1H)-yl]methanone, 5-{[2-(4-Bromophenyl)imidazo[1,2-a]pyridin-3-yl]methyl}-N-methyl-N-phenylhexahydropyrrolo[3,4-c]pyrrole-2(1H)-carboxamide, {[2-(4-Bromophenyl)imidazo[1,2-a]pyridin-3 -yl]methyl}hexahydropyrrolo[3,4-c]pyrrol-2(1H)-yl](3,4-dihydroquinoline-1(2H)-yl)methanone, [5-{[2-(4-Bromophenyl)imidazo[1,2-a]pyridin-3-yl]methyl}hexahydropyrrolo[3,4-c]pyrrol-2(1H)-yl](3,4-dihydroisoquinoline-2(1H)-yl)methanone, Isobutyl 5-{[2-(4-bromophenyl)imidazo[1,2-a]pyridin-3-yl]methyl}hexahydropyrrolo[3,4-c]pyrrole-2(1H)-carboxylate, Benzyl 5-{[2(4-bromophenyl)imidazo[1,2 -a]pyridin-3-yl]methyl}hexahydropyrrolo[3,4-c]pyrrole-2(1H)-carboxylate, Cyclopentyl 5-{[2-(4-bromophenyl)imidazo[1,2-a]pyridin-3-yl]methyl}hexahydropyrrolo [3,4-c]pyrrole-2(1H)-carboxylate, Isopropyl 5-{[2-(4-bromophenyl)imidazo[1,2-a]pyridin-3-yl]methyl}hexahydropyrrolo[3,4-c]pyrrole-2(1H)-carboxylate, 3-(Trifluoromethyl)phenyl 5-{[2-(4-bromophenyl)imidazo[1,2-a]pyridin-3-yl]methyl}hexahydropyrrolo [3,4-c]pyrrole-2(1H)-carboxylate, Fluoroethyl 5-{[2-(4-bromophenyl)imidazo[1,2-a]pyridin-3-yl]methyl}hexahydropyrrolo[3,4-c]pyrrole-2(1H)-carboxylate, 5-{[2-(4-Bromophenyl)imidazo[1,2-a]pyridin-3-yl]methyl}-N-(2,4-difluorophenyl)hexahydropyrrolo[3,4-c]pyrrole-2(1H)-carboxamide, 5-{[2-(4-Bromophenyl)imidazo[1,2-a]pyridin-3-yl]methyl}-N-(2,6-difluorobenzyl)hexahydropyrrolo[3,4-c]pyrrole-2(1H)- carboxamide, 5-{[2-(4-Bromophenyl)imidazo[1,2-a]pyridin-3-yl]methyl}-N-(2,6-dimethylphenyl)hexahydropyrrolo[3,4-c]pyrrole-2(1H)-carboxamide, 5-{[2-(4-Bromophenyl)imidazo[1,2-a]pyridin-3-yl]methyl}-N-(2-fluorophenyl)hexahydropyrrolo[3,4-c]pyrrole-2(1H)- carboxamide, 5-{[2-(4-Bromophenyl)imidazo[1,2-a]pyridin-3-]methyl}-N-(2-ethoxyphenyl)hexahydropyrrolo[3,4-c]pyrrole-2(1H)-carboxamide, 5-{[2-(4-Bromophenyl)imidazo[1,2-a]pyridin-3-yl]methyl}-N-[4-chloro-3-(trifluoromethyl)phenyl]hexahydropyrrolo[3,4-c]pyrrole-2(1H)-carboxamide, 5-{[2-4-Bromophenyl)imidazo[1,2-a]pyridin-3-yl]methyl}-N-[2-chloro-5-(trifluoromethyl)phenyl]hexahydropyrrolo[3,4-c]pyrrole-2(1H)-carboxamide, 5-{[2-(4- Bromophenyl)imidazo[1,2-a]pyridin-3-yl]methyl}-N-(cyclohexyl)hexahydropyrrolo[3,4-c]pyrrole-2(1H)-carboxamide, rac-5-{[2-(4-Bromophenyl)imidazo[1,2-a]pyridin-3-yl]methyl}-N-(1-phenylethyl)hexahydropyrrolo[3,4-c]pyrrole-2(1H)-carboxamide, 5-{[2-(4-Bromophenyl)imidazo[1,2-a]pyridin-3-yl]methyl}-N-(4-fluorophenyl)hexahydropyrrolo[3,4-c]pyrrole-2(1H)-carboxamide, (3-Fluoro-6-methoxypyridin-2-yl)[5-[2-(4-isopropylphenyl)imidazo[1,2-a]pyridin-3-yl]methyl}hexahydropyrrolo[3,4-c]pyrrol-2(1H)-yl]methanone, [5-{[2-(4-Chlorophenyl)imidazo[1,2-a]pyridin-3-yl]methyl}hexahydropyrrolo[3,4-c]pyrrol-2(1H)-yl](6-methoxy-3-methylpyridin-2-yl)methanone, [5-{[2-(4-Chlorophenyl)imidazo[1,2-a]pyridin-3-yl]methyl}hexahydropyrrolo[3,4-c]pyrrol-2(1H)-yl](3-fluoro-6-methoxypyridin-2-yl)methanone, 3-Chloro-6-methoxypyridin-2-yl)[5-{[2-(4-chlorophenyl)imidazo[1,2-a]pyridin-3-yl]methyl}hexahydropyrrolo[3,4-c]pyrrol-2(1H)-yl]methanone, tert-Butyl 5- {[2-(4-chlorophenyl)imidazo[1,2-a]pyridin-3-yl]methyl}-2,5-diazabicyclo[2.2.2]octane-2-carboxylate, tert-Butyl 5-{[2-(5-chloropyridin-2-yl)imidazo[1,2-a]pyridin-3-yl]methyl}-2,5-diazabicyclo[2.2.2]octane-2-carboxylate, tert-Butyl 7-{[2-(4-chlorophenyl)imidazo[1,2-a]pyridin-3-yl]methyl}-3-oxa-7,9- diazabicyclo[3.3.1]nonane-9-carboxylate, tert-Butyl 8-{[2-(4-bromophenyl)imidazo[1,2-a]pyridin-3-yl]methyl-3,8-diazabicyclo[3.2.1]octane-3-carboxylate, tert-Butyl 8-{[2-(4-chlorophenyl)imidazo[1,2-a]pyridin-3-yl]methyl }-3,8-diazabicyclo[3.2.1]octane-3-carboxylate, tert-Butyl 8-[2-(4-isopropylphenyl)imidazo[1,2-a]pyridin-3-yl]methyl}-3,8-diazabicyclo[3.2.1]octane-3-carboxylate, tert-Butyl 3-{[2-(4-chlorophenyl)imidazo[1,2-a]pyridin-3-yl]methyl}-3,6-diazabicyclo[3.1.1]heptane-6-carboxylate, tert-Butyl 3-{[2-(4-isopropylphenyl)imidazo[1,2-a]pyridin-3-yl]methyl}-3,6- diazabicyclo[3.1.1]heptane-6-carboxylate, tert-Butyl 3-{[2-(4-chlorophenyl)imidazo[1,2-a]pyridin-3-yl]methyl}-3,8-diazabicyclo[3.2.1]octane-8-carboxylate, tert-Butyl 3-{[2-(4-bromophenyl)imidazo[1,2-a]pyridine-3-yl]methyl}-3,8-diazabicyclo[3.2.1]octane-8-carboxylate, tert-Butyl 3-{[2-(4-isopropylphenyl)imidazo[1,2-a]pyridin-3-yl]methyl}-3,8-diazabicyclo[3.2.1]octane-8-carboxylate, tert-Butyl 5-{[2-(4-isopropylphenyl)imidazo [1,2-a]pyridin-3-yl]methyl}-2,5-diazabicyclo[2.2.2]octane-2-carboxylate, tert-Butyl 3-{[2-(5-chloropyridin-2-yl)imidazo[1,2-a]pyridin-3-yl]methyl}-3,8-diazabicyclo [3.2.1]octane-8-carboxylate, tert-Butyl 5-{[2-(4-chlorophenyl)imidazo[1,2-a]pyridin-3-yl]methyl}-2,5-diazabicyclo[2.2.2]octane-2-carboxylate, tert-Butyl 5-{[2-(4-isopropylphenyl)imidazo[1,2-a]pyridin-3-yl]methyl}-2,5-diazabicyclo[2.2.2]octane-2-carboxylate, tert-Butyl 5-{[2-(5-chloropyridin-2-yl)imidazo[1,2-a]pyridin-3-yl]methyl}-2,5-diazabicyclo[2.2.2]octane-2-carboxylate, (−)-[(1S,4S)-5-{[2-(4-Chlorophenyl)imidazo[1,2-a]pyridin-3-yl]methyl}-2,5-diazabicyclo[2.2.2]oct-2-yl](6-methoxypyridin-2-yl)methanone, (−)-(3-Chloro-6-methoxypyridin-2-yl)[(1S,4S)-5-{[2-(4-chlorophenyl)imidazo[1,2-a]pyridin-3-yl]methyl}-2,5 -diazabicyclo[2.2.2]oct-2-yl]methanone, (−)-[(1S,4S)-5-{[2-(4-Chlorophenyl)imidazo[1,2-a]pyridin-3-yl]methyl}-2,5-diazabicyclo[2.2.2]oct-2-yl](3-fluoro-6-methoxypyridin-2-yl)methanone, (5-{[2-(5-Chloropyridin-2-yl)imidazo[1,2-a]pyridin-3-yl]methyl}-2,5-diazabicyclo[2.2.2]oct-2-yl)(3-fluoro-6-methoxypyridin-2-yl)methanone, (3-Chloro-6-methoxypyridin-2-yl)(5{[2-(5-chloropyridin-2-yl)imidazo[1,2-a]pyridin-3-yl]methyl}-2,5-diazabicyclo[2.2.2]oct-2-yl)methanone, (−)-(5-{[2-(5-Chloropyridin-2-yl)imidazo[1,2-a]pyridin-3-yl]methyl}-2,5 -diazabicyclo[2.2.2]oct-2-yl)(6-methoxypyridin-2-yl)methanone, (5-{[2-(6-Isopropylpyridin-3-yl)imidazo[1,2 -a]pyridin-3-yl]methyl}-2,5-diazabicyclo[2.2.2]oct-2-yl)(6-methoxypyridin-2-yl)methanone, (3-Fluoro-6-methoxypyridin-2-yl)(5-{[2-(6-isopropylpyridin-3-yl)imidazo[1,2-a]pyridin-3-yl]methyl}-2,5-diazabicyclo[2.2.2]oct- 2-yl)methanone, (7-{[2-(4-Chlorophenyl)imidazo[1,2-a]pyridin-3-yl]methyl}-3-oxa-7,9-diazabicyclo[3.3.1]non-9-yl)(6-methoxypyridin-2-yl)methanone, (7-{[2-(4-Chlorophenyl)imidazo[1,2-a]pyridin-3-yl]methyl}-3-oxa-7,9-diazabicyclo[3.3.1]non-9-yl)(3-fluoro-6-methoxypyridin-2-yl)methanone, (7-{[2-(4-Chlorophenyl)imidazopyridin-3-yl]methyl}-3-oxa-7,9- diazabicyclo[3.3.1]non-9-yl)[6-(cyclobutyloxy)pyridin-2-yl]methanone, (3-Chloro-6-methoxypyridin-2-yl)(7-{[2-(4-chlorophenyl)imidazo[1,2-a]pyridin-3yl]methyl}-oxa-7,9-diazabicyclo[3.3.1]non-9-yl)methanone, (3-{[2-(5-Chloropyridin-2-yl)imidazo[1,2-a]pyridin-3-yl]methyl}-3,8-diazabicyclo[3.2.1]oct-8-yl)(6-methoxypyridin-2-yl)methanone (+)-[(1R,4R)-5-{[2-(4-Chlorophenyl)imidazo[1,2-a]pyridin-3-yl]methyl}-2,5 -diazabicyclo[2.2.2]oct-2-yl](6-methoxypyridin-2-yl)methanone, (−)-(5-{[2-(4-Chlorophenyl)imidazo[1,2-a]pyridin-3-yl]methyl}-2,5-diazabicyclo[2.2.2]oct-2-yl)(2-fluorophenyl)methanone, (+)-(5-{[2-(4-Chlorophenyl)imidazo[1,2-a]pyridin-3-yl]methyl}-2,5-diazabicyclo[2.2.2]oct-2-yl)(2-fluorophenyl)methanone, 5 -{[2-(4-Chlorophenyl)imidazo[1,2-a]pyridin-3-yl]methyl}-2,5-diazabicyclo[2.2.2]oct-2-yl)(cyclopentyl)methanone, (5-{[2-(4-Chlorophenyl)imidazo[1,2-a]pyridin-3-yl]methyl}-2,5-diazabicyclo[2.2.2]oct-2-yl)(cyclopentyl)methanone, (−)-(5-{[2-(4 -Chlorophenyl)imidazo[1,2-a]pyridin-3-yl]methyl}-2,5-diazabicyclo[2.2.2]oct-2-yl)(3-methoxyphenyl)methanone, (+)-(5-{[2-(4-Chlorophenyl)imidazo[1,2-a]pyridin-3-yl]methyl}-2,5-diazabicyclo[2.2.2]oct-2-yl)(3-methoxyphenyl)methanone, (2-Chloro-5-fluorophenyl)(5-{[2-(4-chlorophenyl)imidazo[1,2-a]pyridin-3-yl]methyl -2,5-diazabicyclo[2.2.2]oct-2-yl)methanone, (5-{[2-(4-Chlorophenyl)imidazo[1,2-a]pyridin-3-yl]methyl}-2,5-diazabicyclo[2.2.2]oct-2-yl)(cyclohexyl)methanone, (5-{[2-(4-Chlorophenyl)imidazo[1,2-a]pyridin-3-yl]methyl}-2,5-diazabicyclo[2.2.2]oct-2-yl)(cyclobutyl)methanone, (5-{[2-(4-Chlorophenyl)imidazo[1,2-a]pyridin-3-yl]methyl}-2,5-diazabicyclo[2.2.2]oct-2-yl)(2-methoxyphenyl)methanone, (5-{[2-(4-Chlorophenyl)imidazo[1,2-a]pyridin-3-yl]methyl}-2,5-diazabicyclo[2.2.2]oct-2-yl)(5-fluoro-2-methoxyphenyl)methanone, (5-{[2-(4-Chlorophenyl)imidazo[1,2-a]pyridin-3-yl]methyl}-2,5-diazabicyclo[2.2.2]oct-2-yl)(2-methylphenyl)methanone, (5-{[2-(4-Chlorophenyl)imidazo[1,2-a]pyridin-3-yl]methyl}-2,5-diazabicyclo[2.2.2]oct-2-yl)(5-fluoro-2-methylphenyl)methanone, (5-{[2-(4-Chlorophenyl)imidazo[1,2-a]pyridin-3-2,5-diazabicyclo[2.2.2]oct-2-yl)[3-(trifluoromethoxy)phenyl]methanone, (3-Chlorophenyl)(5-{[2-(4-chlorophenyl)imidazo[1,2-a]pyridin-3-yl]methyl}-2,5-diazabicyclo[2.2.2]oct-2-yl)methanone, (5-{[2-(4-Chlorophenyl)imidazo[1,2-a]pyridin-3 -yl]methyl}-2,5-diazabicyclo[2.2.2]oct-2 -yl)[{4-trifluoromethyl)phenyl]methanone, (5-{[2-(4-Chlorophenyl)imidazo[1,2-a]pyridin-3-yl]methyl}-25-diazabicyclo[2.2.2]oct-2-yl)(pyridin-2-yl]methanone, (5-{[2-(4-Chlorophenyl)imidazo[1,2-a]pyridin-3-yl]methyl}-2,5-diazabicyclo[2.2.2]oct-2-yl)(1-methyl-1H-imidazol-2-yl)methanone, (5-{[2-(4-Chlorophenyl)imidazo[1,2-a]pyridin-3-yl]methyl}-2,5-diazabicyclo[2.2.2]oct-2-yl)(3-methylphenyl)methanone, (5-{[2-(4-Chlorophenyl)imidazo[1,2-a]pyridin-3-yl]methyl}-2,5-diazabicyclo[2.2.2]oct-2-yl)(3-ethoxyphenyl)methanone, (5-{[2-(4-Chlorophenyl)imidazo[1,2-a]pyridin-3-yl]methyl}-2,5-diazabicyclo[2.2.2]oct-2-yl)(pyridin-4-yl)methanone, (−)-(2-Fluorophenyl)(5-{[2-(4-isopropylphenyl)imidazo[1,2-a]pyridin-3-yl]methyl}-2,5-diazabicyclo[2.2.2]oct-2-yl)methanone, (+)-(2-Fluorophenyl)(5-{[2-(4 -isopropylphenyl)imidazo[1,2-a]pyridin-3-yl]methyl}-2,5-diazabicyclo[2.2.2]oct-2-yl)methanone, (−)-(5-[2-(4-Isopropylphenyl)imidazo[1,2-a]pyridin-3-yl]methyl}-2,5-diazabicyclo[2.2.2]oct-2-yl)(3-methoxyphenyl)methanone (+)-(5-{[2-(4-Isopropylphenyl)imidazo[1,2 -a]pyridin-3-yl]methyl}-2,5- diazabicyclo[2.2.2]oct-2-yl)(3-methoxyphenyl)methanone, (−)-(5-{[2-(4-Isopropylphenyl)imidazo[1,2-a]pyridin-3-yl]methyl}-2,5-diazabicyclo[2.2.2]oct-2-yl)(6-methoxypyridin-2-yl)methanone (+)-(5-{[2-(4-Isopropylphenyl)imidazo[1,2-a]pyridin-3-yl]methyl}-2,5-diazabicyclo[2.2.2]oct- 2-yl)(6-methoxypyridin-2-yl)methanone, Cyclopentyl(5-{[2-(4-isopropylphenyl)imidazo[1,2-a]pyridin-3-yl]methyl}-2,5-diazabicyclo[2.2.2]oct-2-yl)methanone, Cyclopentyl(5-{[2-(4-isopropylphenyl)imidazo[1,2-a]pyridin-3-yl]methyl}-2,5-diazabicyclo[2.2.2]oct-2-yl)methanone, (−)-(5-[2-(5-Chloropyridin-2-yl)imidazo[1,2-a]pyridin-3-yl]methyl}-2,5-diazabicyclo[2.2.2]oct-2-yl)(2-fluorophenyl)methanone, (+)-(5-{[2-(5- Chloropyridin-2-yl)imidazo[1,2-a]pyridin-3-yl]methyl}-2,5-diazabicyclo[2.2.2]oct-2-yl)(6-methoxypyridin-2-yl)methanone, (5-{[2-(5-Chloropyridin-2-yl)imidazo[1,2-a]pyridin-3 -yl]methyl}-2,5 -diazabicyclo[2.2.2]oct-2-yl)(3-fluoro-6-methoxypyridin-2-yl)methanone, (3-Chloro-6-methoxypyridin-2-yl)(5-{[2-(5-chloropyridin-2-yl)imidazo[1,2-a]pyridin-3-yl]methyl}-2,5-diazabicyclo[2.2.2]oct-2-yl)methanone, (2-Fluorophenyl)(5-{[2-(6-isopropylpyridin-3-yl)imidazo[1,2-a]pyridin-3-yl]methyl}-2,5- diazabicyclo[2.2.2]oct-2-yl)methanone, (7-{[2-(4-Chlorophenyl)imidazo[1,2-a]pyridin-3-yl]methyl}-3-oxa-7,9-diazabicyclo[3.3.1]non-9-yl)(2-fluorophenyl)methanone, (7-{[2-(4-Chlorophenyl)imidazo[1,2-a]pyridin-3-yl]methyl}-3-oxa-7,9-diazabicyclo[3.3.1]non-9-yl)(3-methoxyphenyl)methanone, (7-{[2-(4-Chlorophenyl)imidazo[1,2-a]pyridin-3-yl]methyl}-3-oxa-7,9-diazabicyclo[3.3.1]non-9-yl)(cyclopentyl)methanone, (7-{[2-(4-Chlorophenyl)imidazo[1,2-a]pyridin-3-yl]methyl}-3-oxa-7,9-diazabicyclo[3.3.1]non-9 -yl)[3-(trifluoromethoxy)phenyl]methanone, (7-{[2-(4-Chlorophenyl)imidazo[1,2-a]pyridin-3-yl]methyl}-3-oxa-7,9-diazabicyclo[3.3.1 ]non-9-yl)(2-isopropylphenyl)methanone, (2-Chloro-5-methoxyphenyl)(7-[2-(4-chlorophenyl)imidazo[1,2-a]pyridin-3-yl]methyl}-3-oxa-7,9-diazabicyclo[3.3.1]non-9-yl)methanone, (7-{[2-(4-Chlorophenyl)imidazo[1,2- a]pyridin-3-yl]methyl}-3-oxa-7,9-diazabicyclo[3.3.1]non-9-yl)(5-fluoro-2-methoxyphenyl)methanone, (7-{[2-(4-Chlorophenyl)imidazo[1,2-a]pyridin-3-yl]methyl}-3-oxa-7,9-diazabicyclo[3.3.1]non-9-yl)(3-isopropylphenyl)methanone, (7-{[2-(4-Chlorophenyl)imidazo[1,2-a]pyridin-3-yl]methyl}-3-oxa-7,9-diazabicyclo[3.3.1]non-9-yl)[6-(2,2,2-trifluoroethoxy)pyridin-2-yl]methanone, (7-{[2-(4-Chlorophenyl)imidazo[1,2-a]pyridin-3-yl]methyl}-3-oxa-7,9- diazabicyclo[3.3.1]non-9-yl)(tetrahydrofuran-3-yl)methanone, (3-Chlorophenyl)(7-{[2-(4-chlorophenyl)imidazo[1,2-a]pyridin-3-yl]methyl}-3-oxa-7,9-diazabicyclo[3.3.1]non-9-yl)methanone, (7-{[2-(4-Chlorophenyl)imidazo[1,2-a]pyridin-3-yl]methyl}-3-oxa-7,9-diazabicyclo[3.3.1]non-9-yl)[6-(trifluoromethoxy)pyridin-2-yl]methanone, (7-{[2-(4-Chlorophenyl)imidazo[1,2-a]pyridin-3-yl]methyl}-3-oxa-7,9-diazabicyclo[3.3.1]non-9-yl)(6-methoxy-3-methylpyridin-2-yl)methanone, (8-{[2-(4-Bromophenyl)imidazo[1,2-a]pyridin-3-yl]methyl}-3,8-diazabicyclo[3.2.1]oct-3-yl)(2-fluorophenyl)methanone, (8-{[2-(4-Bromophenyl)imidazo[1,2-a]pyridin-3-yl]methyl}-3,8-diazabicyclo[3.2.1]oct-3-yl)(6-methoxypyridin-2-yl)methanone, (8-{[2-(4-Bromophenyl)imidazo[1,2-a]pyridin-3-yl]methyl}-3,8-diazabicyclo[3.2.1]oct-3-yl)(3-methoxyphenyl)methanone, (8-{[2-(4-Bromophenyl)imidazo[1,2-a]pyridin-3-yl]methyl}-3,8-diazabicyclo[3.2.1]oct-3-yl)(cyclopentyl)methanone, (8-{[2-(4-Chlorophenyl)imidazo[1,2-a]pyridin-3-yl]methyl}-3,8-diazabicyclo[3.2.1]oct-3-yl)(cyclopentyl)methanone, (8-{[2-(4-Chlorophenyl)imidazo[1,2-a]pyridin-3-yl]methyl}-3,8-diazabicyclo[3.2.1]oct-3-yl)(2-fluorophenyl)methanone, (8-{[2-(4-Chlorophenyl)imidazo[1,2-a]pyridin-3-yl]methyl}-3,8-diazabicyclo[3.2.1]oct-3-yl)(5-fluoro-2-methylphenyl)methanone, (8-{[2-(4-Chlorophenyl)imidazo[1,2-a]pyridin-3-yl]methyl}-3,8-diazabicyclo[3.2.1]oct-3-yl)(5-fluoro-2-methoxyphenyl)methanone, (8-[{2-(4-Chlorophenyl)imidazo[1,2-a]pyridin-3-yl]methyl}-3,8-diazabicyclo[3.2.1]oct-3-yl)(5-fluoro-2-methylphenyl)methanone, (8-{[2-(4-Chlorophenyl)imidazo[1,2-a]pyridin-3-yl]methyl}-3,8-diazabicyclo[3.2.1]oct-3-yl)(2-methoxyphenyl)methanone, (8-{[2-(4-Chlorophenyl)imidazo[1,2-a]pyridin-3-yl]methyl}-3,8-diazabicyclo[3.2.1]oct-3-yl)(6-methoxypyridin-2-yl)methanone, (8-{[2-(4-Chlorophenyl)imidazo[1,2-a]pyridin-3-yl]methyl}-3,8-diazabicyclo[3.2.1]oct-3-yl)(cyclohexyl)methanone, (2-Fluorophenyl)(8-{[2-(4-isopropylphenyl)imidazo[1,2-a]pyridin-3-yl]methyl}-3,8-diazabicyclo[3.2.1]oct-3-yl)methanone, (8-{[2-(4-isopropylphenyl)imidazo[1,2-a]pyridin-3-yl]methyl}-3,8-diazabicyclo[3.2.1]oct-3-yl)(6-methoxypyridin-2-yl)methanone, (3-{[2-(4-Bromophenyl)imidazo[1,2-a]pyridin-3-yl]methyl}-3,8-diazabicyclo[3.2.1]oct-8-yl)(cyclopentyl)methanone, (3-{[2-(4-Bromophenyl)imidazo[1,2-a]pyridin-3-yl]methyl}-3,8-diazabicyclo[3.2.1]oct-8-yl)(2-fluorophenyl)methanone, (3-{[2-(4-Bromophenyl)imidazo[1,2-a]pyridin-3-yl]methyl}-3,8-diazabicyclo[3.2.1]oct-8-yl)(6-methoxypyridin-2-yl)methanone, (3-{[2-(4-Bromophenyl)imidazo[1,2-a]pyridin-3-yl]methyl}-3,8-diazabicyclo[3.2.1]oct-8-yl)(3-methoxyphenyl)methanone, (3-{[2-(4-Chlorophenyl)imidazo[1,2-a]pyridin-3-yl]methyl}-3,8-diazabicyclo[3.2.1 ]oct-8-yl)(2-methylphenyl)methanone, (3-{[2-(4-Chlorophenyl)imidazo[1,2-a]pyridin-3-yl]methyl}-3,8-diazabicyclo[3.2.1]oct-8-yl)(cyclobutyl)methanone, (3-{[2-(4-Chlorophenyl)imidazo[1,2-a]pyridin-3-yl]methyl}-3,8- diazabicyclo[3.2.1]oct-8-yl)(2-fluorophenyl)methanone, (3-{[2(4-Chlorophenyl)imidazo[1,2-a]pyridin-3-yl]methyl}-3,8-diazabicyclo[3.2.1]oct-8-yl)(5-fluoro-2-methoxyphenyl)methanone, (3-{[2-(4-Chlorophenyl)imidazo[1,2-a]pyridin-3-yl]methyl}-3,8-diazabicyclo[3.2.1]oct-8-yl)(6-methoxypyridin-2-yl)methanone, (3-{[2-(4-Chlorophenyl)imidazo[1,2-a]pyridin-3-yl]methyl}-3,8-diazabicyclo[3.2.1]oct-8-yl)(cyclohexyl)methanone, (2-Chloro-5-fluorophenyl)(3-{[2-(4-chlorophenyl)imidazo-3,8-diazabicyclo[3.2.1]oct-8-yl)methanone, (3-{[2-(4-Chlorophenyl)imidazo[1,2-a]pyridin-3-yl]methyl}-3,8-diazabicyclo[3.2.1]oct-8-yl)(5-fluoro-2-methylphenyl)methanone, (3-{[2-(4- Chlorophenyl)imidazo[1,2-4]pyridin-3-yl]methyl}-3,8-diazabicyclo[3.2.1]oct-8-yl)(3-methoxyphenyl)methanone, (3-{[2-(4-Chlorophenyl)imidazo[1,2-a]pyridin-3-yl]methyl}-3,8-diazabicyclo[3.2.1]oct-8-yl)(2-methoxyphenyl)methanone, (2-Fluorophenyl)(3-[2-(4-isopropylphenyl)imidazo[1,2-a]pyridin-3-yl]methyl}-3,8-diazabicyclo[3.2.1]oct-8-yl)methanone, (3-{[2-(5-Chloropyridin-2-yl)imidazo[1,2-a]pyridin-3-yl]methyl}-3,8-diazabicyclo[3.2.1]oct-8-yl)(2-fluorophenyl)methanone, (3-{[2-(5-Chloropyridin-2-yl)imidazo[1,2-a]pyridin-3-yl]methyl}-3,8- diazabicyclo[3.2.1]oct-8-yl)(3-methoxyphenyl)methanone, (3-{[2-(5-Chloropyridin-2-yl)imidazo[1,2-a]pyridin-3-yl]methyl}-3,8-diazabicyclo[3.2.1]oct-8-yl)(cyclopentyl)methanone, (3-Chlorophenyl)(3-{[2-(4-chlorophenyl)imidazo[1,2-a]pyridin-3-yl]methyl}-3,6-diazabicyclo[3.1.1]hept-6-yl)methanone, (3-{[2-(4-Chlorophenyl)imidazo[1,2-a]pyridin-3-yl]methyl}-3,6-diazabicyclo[3.1.1]hept-6-yl)(tetrahydrofuran-2-yl)methanone, (3-{[2-(4-Chlorophenyl)imidazo[1,2-a]pyridin-3-yl]methyl}-3,6- diazabicyclo[3.1.1]hept-6-yl)(cyclopentyl)methanone, (3-{[2-(4-Chlorophenyl)imidazo[1,2-a]pyridin-3-yl]methyl}-3,6-diazabicyclo[3.1.1]hept-6-yl)(2-fluorophenyl)methanone, (3-{[2-(4-Chlorophenyl)imidazo[1,2-a]pyridin-3-yl]methyl}-3,6-diazabicyclo[3.1.1]hept-6-yl)(cyclohexyl)methanone, (2-Chloro-5-fluorophenyl)(3-{[2-(4-chlorophenyl)imidazo[1,2-a]pyridin-3-yl]methyl}-3,6-diazabicyclo[3.1.1]hept-6-yl)methanone, (3-{[2-(4-Chlorophenyl)imidazo[1,2-a]pyridin-3-yl]methyl}-3,6-diazabicyclo[3.1.1]-hept-6-yl)[3-(trifluoromethoxy)phenyl]methanone, (3-{[2-(4-Chlorophenyl)imidazo[1,2-a]pyridin-3-yl]methyl}-3,6-diazabicyclo[3.1.1]hept-6-yl)(cyclobutyl)methanone, (3{[2-(4-Chlorophenyl)imidazo[1,2-a]pyridin-3-yl]methyl}-3,6-diazabicyclo[3.1.1]hept-6-yl)(3-ethoxyphenyl)methanone, Cyclopentyl(3-{[2-(4-isopropylphenyl)imidazo[1,2-a]pyridin-3-yl]methyl}-3,6-diazabicyclo[3.1.1]hept-6-yl)methanone, (5-{[2-(6-isopropylpyridin-3-yl)imidazo[1,2-a]pyridin-3-yl]methyl}-2,5-diazabicyclo[2.2.2]oct-2-yl)(6-methoxypyridin-2-yl)methanone, (3-Fluoro-6-methoxypyridin-2-yl)(5-{[2-(6-isopropylpyridin-3-yl)imidazo[1,2-a]pyridin-3-yl]methyl}-2,5-diazabicyclo[2.2.2]oct-2-yl)methanone, (2-Fluorophenyl)(5-{[2-(6-isopropylpyridin-3-yl)imidazo[1,2-a]pyridin-3-yl]methyl}-2,5-diazabicyclo[2.2.2]oct-2-yl)methanone, tert-Butyl 7-{[2-(5-chloropyridin-2-yl)imidazo[1,2-a]pyridine-3-yl]methyl}-3-oxa-7,9-diazabicyclo[3.3.1 nonane-9-carboxylate, tert-Butyl 3-{[2-(6-isopropylpyridin-3-yl)imidazo[1,2-a]pyridine-3-yl]methyl}-3,8-diazabicyclo[3.2.1]octane-8-carboxylate, tert-Butyl 5-{[2-(4-bromophenyl)imidazo[1,2-a]pyridin-3-yl]methyl}-2,5-diazabicyclo[2.2.2]octane-2-carboxylate, tert-Butyl 5-{[2-(6-isopropylpyridin-3-yl)imidazo[1,2-a]pyridin-3-yl]methyl}-2,5-diazabicyclo[2.2.2]octane-2-carboxylate, (7-{[2-(5-Chloropyridin-2-yl)imidazo[1,2-a]pyridin-3-yl]methyl}-3-oxa-7,9-diazabicyclo[3.3.1]non-9-yl)[6-(trifluoromethoxy)pyridin-2-yl]methanone, (3-Chloro-6-methoxypyridin-2-yl)(7-{[2-(5-chloropyridin-2-yl)imidazo[1,2-a]pyridin-3-yl]methyl}-3-oxa-7,9-diazabicyclo[3.3.1]non-9-yl)methanone, 5-{[2-(4-Isopropylphenyl)imidazo[1,2-a]pyridin-3-yl]methyl}-2,5-diazabicyclo[2.2.2]oct-2-yl](6-methoxy-3-methylpyridin-2 -yl)methanone, 5-{[2-(4-Chlorophenyl)imidazo[1,2-a]pyridin-3-yl]methyl}-2,5-diazabicyclo[2.2.2]oct-2-yl](6-methoxy-3-methylpyridin-2-yl)methanone, (3-{[2-(4-Bromophenyl)imidazo[1,2-a]pyridin-3-yl]methyl}-3,8-diazabicyclo[3.2.1]oct-8-yl)(3-chloro-6-methoxypyridin-2-yl)methanone, (3-{[2-(4-Bromophenyl)imidazo[1,2-a]pyridin-3-yl]methyl}-3,8-diazabicyclo[3.2.1]oct-8-yl)(3-fluoro-6-methoxypyridin-2-yl)methanone, (3-{[2-(4-Bromophenyl)imidazo[1,2-a]pyridin-3-yl]methyl}-3,8-diazabicyclo[3.2.1oct8-yl)(2- isopropylphenyl)methanone, (3-{[2-(4-Chlorophenyl)imidazo[1,2-a]pyridin-3-yl]methyl}-3,6-diazabicyclo[3.1.1.]hept-6-yl)(6-methoxy-3-methylpyridin-2-yl)methanone, (8-{[2-(4-Chlorophenyl)imidazo[1,2-a]pyridin-3-yl]methyl}-3,8-diazabicyclo[3.2.1]oct-3-yl)(6-methoxy-3-methylpyridin-2-yl)methanone, (8-{[2-(4-Isopropylphenyl)imidazo[1,2-a]pyridin-3-yl]methyl}-3,8-diazabicyclo[3.2.1]oct-3-yl)(6-methoxy-3-methylpyridin-2-yl)methanone, (3-{[2-(4-Isopropylphenyl)imidazo[1,2-a]pyridin-3-yl]methyl}-3,8-diazabicyclo[3.2.1]oct-8-yl)(6-methoxy-3-methylpyridin-2-yl)methanone, (3-{[2-(4-Chlorophenyl)imidazo[1,2-a]pyridin-3-yl]methyl}-3,8-diazabicyclo[3.2.1]oct-8-yl)(4-isopropyl-1,3-thiazol-2-yl)methanone, (3-{[2-(4-Chlorophenyl)imidazo[1,2-a]pyridin-3-yl]methyl}-3,8-diazabicyclo[3.2.1]oct-8-yl)(1,3-thiazol-2-yl)methanone, (3-{[2-(4-Chlorophenyl)imidazo[1,2-a]pyridin-3-yl]methyl}-3,8-diazabicyclo[3.2.1]oct-8-yl)(4-methyl-1,3-thiazol-2-yl)methanone, (3-{[2-(4-Chlorophenyl)imidazo[1,2-a]pyridin-3-yl]methyl}-3,8-diazabicyclo[3.2.1]oct-8-yl)(5-methyl-1,3-thiazol-2-yl)methanone, (3{[2-(4-Chlorophenyl)imidazo[1,2-a]pyridin-3-yl]methyl}-3,8-diazabicyclo[3.21]oct-8-yl)(4,5-dimethyl-1,3-thiazol-2-yl)methanone, (5-{[2(4-Bromophenyl)imidazo[1,2-a]pyridin-3-yl]methyl}-2,5-diazabicyclo[2.2.2]oct-2-yl)(6-methoxypyridin-2-yl)methanone, (5-{[2-(4-Bromophenyl)imidazo[1,2-a]pyridin-3-yl]methyl}-2,5-diazabicyclo[2.2.2]oct-2-yl)(2-fluorophenyl)methanone, (5-{[2-(4-Bromophenyl)imidazo[1,2-a]pyridin-3-yl]methyl}-2,5-diazabicyclo[2.2.2 ]oct-2 -yl)(3-fluoro-6-methoxypyridin-2 -yl)methanone, 3- {[2-(4-Chlorophenyl)imidazo[1,2-a]pyridin-3-yl]methyl}-N-isopropyl-3,8-diazabicyclo[3.2.1]octane-8-carboxamide, 3-{[2-(4-Chlorophenyl)imidazo[1,2-a-]pyridin-3-yl]methyl}-N-(2-fluorophenyl)-3,8-diazabicyclo[3.2.1 ]octane-8-carboxamide, 3-{[2-(4-Chlorophenyl)imidazo[1,2-a]pyridin-3-yl]methyl -N-(2,6- dichlorophenyl)-3,8-diazabicyclo[3.2.1 ]octane-8-carboxamide, 3{[2-(4-Chlorophenyl)imidazo[1,2-a]pyridin-3-yl]methyl}-N-(2,6-dimethylphenyl)-3,8-diazabicyclo[3.2.1]octane-8-carboxamide, 3-{[2-(4-Chlorophenyl)imidazo[1,2-a]pyridin-3-yl]methyl}-N-pentyl-3,8-diazabicyclo[3.2.1]octane-8-carboxamide, 3{[2-(4-Chlorophenyl)imidazo[1,2-a]pyridin-3-yl]methyl}-N-(2-methylphenyl)-3,8-diazabicyclo[3.2.1]octane-8-carboxamide, 3-{[2-(4-Chlorophenyl)imidazo[1,2-a]pyridin-3-yl]methyl}-N-(2-chloro-5-(trifluoromethyl)phenyl]-3,8-diazabicyclo[3.2.1]octane-8-carboxamide, N-(4-Chlorophenyl)-3{[2-(4-chlorophenyl)imidazo[1,2-a]pyridin-3-yl]methyl}-3,8-diazabicyclo[3.2.1]octane-8-carboxamide, 3-{[2-(4-Chlorophenyl)imidazo[1,2-a]pyridin-3-yl]methyl}-N-(2-ethyl-6-methylphenyl)-3,8-diazabicyclo[3.2.1]octane-8-carboxamide, 3-{[2-(4-Chlorophenyl)imidazo[1,2-a]pyridin-3-yl]methyl}-N-(2,5- dimethylphenyl)-3,8-diazabicyclo[3.2.1]octane-8-carboxamide, 3{[2-(4-Chlorophenyl)imidazo[1,2-a]pyridin-3-yl]methyl}-N-cyclohexyl-3,8-diazabicyclo[3.2.1]octane-8-carboxamide, N-(2-Chloro-6-methylphenyl)-3-{[2-(4-chlorophenyl)imidazo[1,2-a]pyridin-3-yl]methyl}-3,8-diazabicyclo[3.2.1]octane-8-carboxamide, 3-{[2-(4-Chlorophenyl)imidazo[1,2-a]pyridin-3-yl]methyl}-N-(2,6-difluorophenyl)-3,8-diazabicyclo[3.2.1]octane-8-carboxamide, 3-{[2-(4-Chlorophenyl)imidazo[1,2-a]pyridin-3-yl]methyl}-N-(2,4-dimethylphenyl)-3,8-diazabicyclo[3.2.1]octane-8-carboxamide, 7-{[2-(4-Chlorophenyl)imidazo[1,2-a]pyridin-3-yl]methyl}-N-isopropyl-3-oxa-7,9- diazabicyclo[3.3.1]nonane-9-carboxamide, N-(2-Chloro-6-methylphenyl)-7-[2-(4-chlorophenyl)imidazo[1,2-a]pyridin-3-yl]methyl}-3-oxa-7,9-diazabicyclo[3.3.1]nonane-9-carboxamide, 3{[2-(4-Chlorophenyl)imidazo[1,2-a]pyridin-3-yl]methyl}-N-cyclopropyl-3,8-diazabicyclo[3.2.1]octane-8-carboxamide, N-(2-Chlorophenyl)-3{[2-(4-chlorophenyl)imidazo[1,2-a]pyridin-3-yl]methyl}-3,8- diazabicyclo[3.2.1]octane-8-carboxamide, 3-{[2-(4-Chlorophenyl)imidazo[1,2-a]pyridin-3-yl]methyl}-N-methyl-N-phenyl-3,8-diazabicyclo[3.2.1]octane-8-carboxamide, (3{[2-(4-Chlorophenyl)imidazo[1,2-a]pyridin-3-yl]methyl}-3,8-diazabicyclo[3.2.1]oct-8-yl)(morpholin-4-yl)methanone, 3-{[2-(4-Chlorophenyl)imidazo[1,2-a]pyridin-3-yl]methyl}-N,N-diisopropyl-3,8-diazabicyclo[3.2 ]octane-8-carboxamide, 3-{[2-(4-Chlorophenyl)imidazo[1,2-a]pyridin-3-yl]methyl}-N-cyclohexyl- N-ethyl-3,8-diazabicyclo[3.2.1]octane-8-carboxamide, (3-{[2-(4-Chlorophenyl)imidazo[1,2-a]pyridin-3-yl]methyl}-3,8-diazabicyclo[3.2.1]oct-8-yl)(pyrrolidin-1-yl)methanone, 3-{[2-(4-Chlorophenyl)imidazo[1,2-a]pyridin-3-yl]methyl}-N-ethyl-N-phenyl-3,8-diazabicyclo[3.2.1]octane-8-carboxamide, 3-{[2-(4-Chlorophenyl)imidazo[1,2-a]pyridin-3-yl]methyl}-N-isopropyl-N-methyl-3,8-diazabicyclo[3.2.1]octane-8-carboxamide, (3-{[2-(4-Chlorophenyl)imidazo[1,2-a]pyridin-3-yl]methyl}-3,8-diazabicyclo[3.2.1]oct-8-yl)(piperidin-1-yl)methanone, 3-{[2-(4-Chlorophenyl)imidazo[1,2-a]pyridin-3-yl]methyl}N-dimethyl-3,8-diazabicyclo[3.2.1]octane-8-carboxamide, 3-{[2-(4-Chlorophenyl)imidazo[1,2-a]pyridin-3-yl]methyl}-N-ethyl-N-(4-methylphenyl)-3,8-diazabicyclo[3.2.1]octane-8-carboxamide, N-(4-Chlorophenyl)-3-{[2-(4-chlorophenyl)imidazo[1,2-a]pyridin-3-yl]methyl}-N-isopropyl-3,8-diazabicyclo[3.2.1]octane-8-carboxamide, (3-{[2-(4-Chlorophenyl)imidazo[1,2-a]pyridin-3-yl]methyl}-3,8-diazabicyclo[3.2.1]oct-8-yl)(thiomorpholin-4-yl)methanone, Methyl 3-{[2-(4-chlorophenyl)imidazo[1,2-a]pyridin-3-yl]methyl}-3,8-diazabicyclo[3.2.1]octane-8-carboxylate, Ethyl 3-{[2-(4-chlorophenyl)imidazo[1,2-a]pyridin-3-yl]methyl}-3,8-diazabicyclo[3.2.1]octane-8-carboxylate, Cyclopentyl 3-{[2-(4-chlorophenyl)imidazo[1,2-a]pyridin-3-yl]methyl}-3,8-diazabicyclo[3.2.1]octane-8-carboxylate, Cyclohexyl 3-{[2-(4-chlorophenyl)imidazo[1,2-a]pyridin-3-yl]methyl}-3,8-diazabicyclo[3.2.1]octane-8-carboxylate, 7-{[2-(4-Chlorophenyl)imidazo[1,2-a]pyridin-3-yl]methyl}N-diethyl-3-oxa-7,9-diazabicyclo[3.3.1]nonane-9-carboxamide, (7-{[2-(4-Chlorophenyl)imidazo[1,2-a]pyridin-3-yl]methyl}-3-oxa-7,9-diazabicyclo[3.3.1]non-9-yl)(morpholin-4-yl)methanone, 7-{[2-(4-Chlorophenyl)imidazo[1,2-a]pyridin-3-yl]methyl}-N,N-diisopropyl-3-oxa-7,9-diazabicyclo[3.3.1]nonane-9-carboxamide 7-{[2-(4-Chlorophenyl)imidazo[1,2-a]pyridin-3-yl]methyl}-N- cyclohexyl-N-ethyl-3-oxa-7,9-diazabicyclo[3.3.1]nonane-9-carboxamide, (7-{[2-(4-Chlorophenyl)imidazo[1,2-a]pyridin-3-yl]methyl}-3-oxa-7,9-diazabicyclo[3.3.1]non-9-yl)(pyrrolidin-1-yl)methanone, 7-{[2-(4-Chlorophenyl)imidazo[1,2-a]pyridin-3-yl]methyl}-N-ethyl-N-phenyl-3-oxa-7,9-diazabicyclo[3.3.1]nonane-9-carboxamide, 7-{[2-(4-Chlorophenyl)imidazo[1,2-a]pyridin-3-yl]methyl}-N-isopropyl-N-methyl-3-oxa-7,9-diazabicyclo[3.3.1]nonane-9-carboxamide, Ethyl 7-{[2-(4-chlorophenyl)imidazo[1,2-a]pyridin-3-yl]methyl}-3-oxa-7,9-diazabicyclo[3.3.1]nonane-9-carboxylate, Cyclopentyl 7-{[2-(4-chlorophenyl)imidazo[1,2-a]pyridin-3-yl]methyl}-3-oxa-7,9- diazabicyclo[3.3.1]nonane-9-carboxylate, Propyl 7-{[2-(4-chlorophenyl)imidazo[1,2-a]pyridin-3-yl]methyl}-3-oxa-7,9-diazabicyclo[3.3.1]nonane-9-carboxylate, (7-{[2-(4-Chlorophenyl)imidazo[1,2-a]pyridin-3-yl]methyl}-3-oxa-7,9-diazabicyclo[3.3.1]non-9-yl)(piperidin-1-yl)methanone, (5-{[2-(4-Bromophenyl)imidazo[1,2-a]pyridin-yl]methyl}-2,5-diazabicyclo[2.2.2]oct-2-yl)(3-chlor-6-methoxypyridin-2-yl)methanone, (5-{[2-(5-Chloropyridin-2-yl)imidazo[1,2-a]pyridin-3-yl]methyl}-2,5-diazabicyclo[2.2.2.]oct-2-yl)[6-(difluoromethoxy)pyridin-2-yl]methanone, tert-Butyl 7-{[2-(4- chlorophenyl)imidazo[1,2-a]pyrimidin-3-yl]methyl}-3-oxa-7,9-diazabicyclo[3.3.1]nonane-9-carboxylate, tert-Butyl 7-[{2-(4-isopropylphenyl)imidazo[1,2-a]pyrimidin-3-yl]methyl}-3-oxa-7,9- diazabicyclo[3.3.1]nonane-9-carboxylate, tert-Butyl 5-{[1-(4-chlorophenyl)imidazo[1,2-a]pyrimidin-3-yl]methyl}-2,5-diazabicyclo[2.2.2]octane-2-carboxylate, tert-Butyl 5-{[2-(4-chlorophenyl)imidazo[1,2-a]pyrimidin-3-yl]methyl}-2,5-diazabicyclo[2.2.2]octane-2-carboxylate, tert-Butyl 3-{[2-(4-chlorophenyl)imidazo[1,2-a]pyridin-3-yl]methyl}-3,8-diazabicyclo[3.2.1]octane-8-carboxylate, tert-Butyl 5-{[2-(4-isopropylphenyl)imidazo[1,2-a]pyrimidine-3-yl]methyl}-2,5- diazabicyclo[2.2.2]octane-2-carboxylate, tert-Butyl 5-{[2(4-isopropylphenyl)imidazo[1,2-a]pyrimidin-3-yl]methyl}-2,5-diazabicyclo[2.2.2]octane-2-carboxylate, tert-Butyl 3-{1-[2-(4-chlorophenyl)imidazo[1,2-a]pyrimidin-3-yl]ethyl}-3,8-diazabicyclo[3.2.1]octane-8-carboxylate, tert-butyl 5-{[2-(4-bromophenyl)imidazo[1,2-a]pyrimidin-3-yl]methyl-2,5-diazabicyclo[2.2.2]octane-2-carboxylate, tert-butyl 3-{[2-(4-bromophenyl)imidazo[1,2-a]pyrimidin-3-yl]methyl}-3,8- diazabicyclo[3.2.1]octane-8-carboxylate, (7-[2-(4-Chlorophenyl)imidazo[1,2-a]pyrimidin-3-yl]methyl}-3-oxa-7,9-diazabicyclo[3.3.1]non-9-yl)(6-methoxypyridin-2-yl)methanone, (3-Chloro-6-methoxypyridin-2-yl)(7{[2-(4-chlorophenyl)imidazo[1,2-a]pyrimidin-3-yl]methyl}-3-oxa-7,9-diazabicyclo[3.3.1]non-9-yl)methanone, (7-{[2-(4-Chlorophenyl)imidazo[1,2-a]pyrimidin-3-yl]methyl}-3-oxa-7,9-diazabicyclo[3.3.1]non-9-yl)(3-fluoro-6-methoxypyridin-2-yl)methanone, (7-{[2-(4-Chlorophenyl)imidazo[1,2-a]pyrimidin-3-yl]methyl}-3-oxa-7,9-diazabicyclo[3.3.1]non-9-yl)[6-(methylsulfanyl)pyridin-2-yl]methanone, (7-{[2-(4-Chlorophenyl)imidazo[1,2-a]pyrimidin-3-yl]methyl}-3-oxa-7,9-diazabicyclo[3.3.1]non-9-yl)(cyclopentyl)methanone, (3-Fluoro-6-methoxypyridin-2-yl)(7-{[2-(4-isopropylphenyl)imidazo[1,2-a]pyrimidin-3-yl]methyl}-3-oxa-7,9-diazabicyclo[3.3.1]non-9-yl)methanone,[6-(Difluoromethoxy)pyridin-2-yl](7-{[2-(4-isopropylphenyl)imidazo[1,2- a]pyrimidin-3-yl]methyl}-3-oxa-7,9-diazabicyclo[3.3.1]non-9-yl)methanone, (3-{[2-(4-Chlorophenyl)imidazo[1,2-a]pyrimidin-3-yl]methyl}-3,8-diazabicyclo[3.2.1]oct-8-yl)(6-methoxypyridin-2-yl)methanone, (3-Chloro-6-methoxypyridin-2 -yl)(3{[2-(4-chlorophenyl)imidazo[1,2-a]pyrimidin-3-yl]methyl}-3,8-diazabicyclo[3.2.1]oct-8-yl)methanone, (3-{[2-(4-Chlorophenyl)imidazo[1,2-a]pyrimidin-3-yl]methyl}-3,8-diazabicycl[3.2.1]oct-8-yl)(3-fluoro-6-methoxypyridin-2-yl)methanone (3-Chloro-6-methoxypyridin-2-yl)(5-{[2-(4-chlorophenyl)imidazo[1,2-a]pyrimidin-3-yl]methyl}-2,5-diazabicyclo[2.2.1]oct-2-yl)methanone, (5-{[2-(4-Chlorophenyl)imidazo[1,2-a]pyrimidin-3-yl]methyl}-2,5-diazabicyclo[2.2.2]oct-2-yl)(3-fluoro-6-methoxypyridin-2-yl)methanone (5-{[2-(4-Chlorophenyl)imidazo[1,2-a]pyrimidin-3-yl]methyl}-2,5- diazabicyclo[2.2.2]oct-2-yl)(6-methoxy-3-methylpyridin-2-yl)methanone, (3-Chloro-6-methoxypyridin-2-yl)(5-{[2-(4-isopropylphenyl)imidazo[1,2-a]pyrimidin-3-yl]methyl}-2,5-diazabicyclo[2.2.2]oct-2- yl)methanone, (5-Cyclopropyl-1,3-oxazol-4-yl)(5-{[2-(4-isopropylphenyl)imidazo[1,2-a]pyrimidin-3-yl]methyl}-2,5-diazabicyclo[2.2.2]oct-2-yl)methanone, (3-Fluoro-6-methoxypyridin-2-yl)(3-{[2-(4-isopropylphenyl)imidazo[1,2-a]pyrimidin-3-yl]methyl}-3,8-diazabicyclo[3.2.1]oct-8-yl)methanone, (3-Chloro-6-methoxypyridin-2-yl)(3-{[2-(4-isopropylphenyl)imidazo[1,2 -a]pyrimidin-3-yl]methyl}-3,8-diazabicyclo[3.2.1]oct-8-yl)methanone, (7-{[2-(4-chlorophenyl)imidazo[1,2-a]pyrimidin-3-yl]methyl}-3-oxa-7,9-diazabicyclo[3.2.1]non-9-yl)(2-fluorophenyl)methanone, (7-{[2-(4-chlorophenyl)imidazo[1,2-a]pyrimidin-3-yl]methyl}-3-oxa-7,9-diazabicyclo[3.3.1]non-9-yl)(3-methoxyphenyl)methanone, (3-{[2-(4-chlorophenyl)imidazo[1,2-a]pyrimidin-3-yl]methyl}-3,8-diazabicyclo[3.2.1]oct-8-yl)(2-fluorophenyl)methanone, (3-{[2-(4-chlorophenyl)imidazo[1,2- a]pyrimidin-3-yl]methyl}-3,8-diazabicyclo[3.2.1 ]oct-8-yl)[6-(methylsulfanyl)pyridin-2-yl]methanone, (3-{[2-(4-chlorophenyl)imidazo[1,2a]pyrimidin-3-yl]methyl}-3,8-diazabicyclo[3.2.1]oct-8-yl)(cyclopentyl)methanone, (3-{[2-(4-chlorophenyl)imidazo[1,2-a]pyrimidin-3-yl]-methyl}-3,8-diazabicyclo[3.2.1]oct-8-yl)[6-(methylamino)pyridin-2-yl]methanone, (3-{[2-(4-chlorophenyl)imidazo[1,2-a]pyrimidin-3-yl]methyl}-3,8-diazabicyclo[3.2.1]oct-8-yl)(3-methoxyphenyl)methanone, (5-{[2-(4-chlorophenyl)imidazo[1,2-a]pyrimidin-3-yl]methyl}-2,5-diazabicyclo[2.2.2]oct-2-yl)(cyclopentyl)methanone, (5-{[2(4-chlorophenyl)imidazo[1,2-a]pyrimidin-3-yl]methyl}-2,5-diazabicyclo[2.2.2]oct-2-yl)(3-methoxyphenyl)methanone, (5-{[2-(4-chlorophenyl)imidazo[1,2-a]pyrimidin-3-yl]methyl}-2,5-diazabicyclo[2.2.2]oct-2-yl)(2-fluorophenyl)methanone, (5-{[2-(4-chlorophenyl)imidazo[1,2-a]pyrimidin-3-yl]methyl}-2,5-diazabicyclo[2.2.2]oct-2-yl)(6-methoxypyridin-2-yl)methanone, (5-{[2-(4-chlorophenyl)imidazo[1,2-a]pyrimidin-3-yl]methyl}-2,5-diazabicyclo[2.2.2]oct-2-yl)(3-fluoro-6-methoxypyridin-2-yl)methanone, (5-{[2-(4-chlorophenyl)imidazo[1,2-a]pyrimidin-3-yl]methyl}-2,5-diazabicyclo[2.2.2]oct-2-yl)(2-fluorophenyl)methanone, (5-{[2-(4- chlorophenyl)imidazo[1,2-a]pyrimidin-3-yl]methyl}-2,5-diazabicyclo[2.2.2]oct-2-yl)(3-methoxyphenyl)methanone, (5-{[2-(4-chlorophenyl)imidazo[1,2-a]pyrimidin-3-yl]methyl}-2,5-diazabicyclo[2.2.2]oct-2-yl)(6-methoxypyridin-2-yl)methanone, (3- chloro-6-methoxypyridin-2-yl)(5-{[2-(4-chlorophenyl)imidazo[1,2-a]pyrimidin-3-yl]methyl}-2,5-diazabicyclo[2.2.2]oct-2-yl)methanone, (7-{[2-(4-chlorophenyl)imidazo[1,2-a]pyrimidin-3-yl]methyl}-3-oxa-7,9-diazabicyclo[3.3.1]non-9-yl)(5-cyclopropyl-1,3-oxazol-4-yl)methanone, (3-{[2-(4-chlorophenyl)imidazo[1,2-a]pyrimidin-3-yl]methyl}-3,8-diazabicyclo[3.2.1]oct-8-yl)(2-cyclopropyl-1,3-oxazol-4-yl)methanone, (3-{[2-(4-chlorophenyl)imidazo[1,2-a]pyrimidin-3-yl]methyl}-3,8-diazabicyclo[3.2.1]oct-8-yl)(5-methyl-1,3-oxazol-4-yl)methanone, (3-{[2-(4-chlorophenyl)imidazo[1,2-a]pyrimidin-3-yl]methyl}-3,8-diazabicyclo[3.2.1]oct-8-yl)(5-isopropyl-1,3-oxazol-4-yl)methanone, (3-{[2-(4-chlorophenyl)imidazo[1,2-a]pyrimidin-3-yl]methyl}-3,8-diazabicyclo[3.2.1]oct-8-yl)(2,4-dimethyl-1,3-oxazol-5-yl)methanone, (3-{[2-(4-chlorophenyl)imidazo[1,2-a]pyrimidin-3-yl]methyl}-3,8-diazabicyclo[3.2.1]oct-8-yl)(5-ethyl-1,3-oxazol-4-yl)methanone, (4-bromo-5-methyl-1,3-thiazol-2-yl)(3-{[2-(4-chlorphenyl)imidazo[1,2-a]pyrimidin-3-yl]methyl}-3,8-diazabicyclo[3.2.1]oct-8-yl)methanone, (3-{[2-(4-chlorophenyl)imidazo[1,2-a]pyrimidin-3-yl]methyl}-3,8-diazabicyclo[3.2.1]oct-8-yl)(5-cyclopropyl-1,3-oxazol-4-yl)methanone, (3-{[2-(4-chlorophenyl)imidazo[1,2-a]pyrimidin-3-yl]methyl}-3,8-diazabicyclo[3.2.1]oct-8-yl)(2-isopropyl-1,3-thiazol-4-yl)methanone, (3-{[2-(4-chlorophenyl)imidazo[1,2-a]pyrimidin-3-yl]methyl}-3,8-diazabicyclo[3.2.1]oct-8-yl)(1,3-thiazol-5-yl)methanone, (3-{[2-(4-chlorophenyl)imidazo[1,2-a]pyrimidin-3 -yl]methyl}-3,8-diazabicyclo[3.2.1]oct-8-yl)(2,5-dimethyl-1,3-oxazol-4-yl)methanone, (3-{[2-(4-chlorophenyl)imidazo[1,2-a]pyrimidin-3-yl]methyl}-3,8-diazabicyclo[3.2.1]oct-8-yl)[2-methoxy-4-(trifluoromethyl)-1,3-thiazol-5-yl)methanone, (3-{[2-(4-chlorophenyl)imidazo[1,2-a]pyrimidin-3-yl]methyl}-3,8-diazabicyclo[3.2.1]oct-8-yl)[2-(trifluoromethyl)-1,3-thiazol-4-yl]methanone, (3-{[2-(4-chlorophenyl)imidazo[1,2-a]pyrimidin-3-yl]methyl}-3,8-diazabicyclo[3.2.1]oct-8-yl)(5-methyl-1,3-thiazol-4-yl)methanone, (3-{[2-(4-chlorophenyl)imidazo[1,2-a]pyrimidin-3-yl]methyl}-3,8-diazabicyclo[3.2.1]oct-8-yl)[4-(trifluoromethyl)-1,3-thiazol-2-yl]methanone, (3-{[1-(4-chlorophenyl)imidazo[1,2a]pyrimidin-3-yl]methyl}-3,8-diazabicyclo[3.2.1]oct-8-yl)(1,3-thiazol-4-yl)methanone, (3-{[2-(4-isopropylphenyl)imidazo[1,2-a]pyrimidin-3-yl]methyl}-3,8-diazabicyclo[3.2.1]oct-8-yl)[6-(methylamino)pyridin-2-yl]methanone, (3-{[2-(4-isopropylphenyl)imidazo[1,2-a]pyrimidin-3-yl]methyl}-3,8-diazabicyclo[3.2.1]oct-8-yl)(6-methoxypyridin-2-yl)methanone, (2-fluorophenyl)(3-{[2-(4-isopropylphenyl)imidazo[1,2-a]pyrimidin-3-yl]methyl}-3,8-diazabicyclo[3.2.1]oct-8-yl)methanone, (3-{1-[2-(4-chlorophenyl)imidazo[1,2-a]pyrimidin-3-yl]ethyl}-3,8-diazabicyclo[3.2.1]oct-8-yl)(3-fluoro-6-methoxypyridin-2-yl)methanone, (7-{[2-(4-isopropylphenyl)imidazo[1,2-a]pyrimidin-3-yl]methyl}-3-oxa-7,9-diazabicyclo[3.3.1]non-9-yl)(6-methoxypyridin -2-yl)methanone, (3-chloro-6-methoxypyridin-2-yl)(7-{[1-(4-isopropylphenyl)imidazo[1,2-a]pyrimidin-3-yl]methyl{-3-oxa-7,9- diazabicyclo[3.3.1]non-9-yl)methanone, (2-fluorophenyl)(7-{[2-(4-isopropylphenyl)imidazo[1,2-a]pyrimidin-3-yl]methyl -3-oxa-7,9-diazabicyclo[3.3.1]non-9-yl)methanone, (3-chloro-6-methoxypyridin-2-yl)(5-[2-(4-isopropylphenyl)imidazo[1,2-a]pyrimidin-3-yl]methyl}-2,5- diazabicyclo[2.2.2]oct-2-yl)methanone, (5-cyclopropyl-1,3-oxazol-4-yl)(5-[2-(4-isopropylphenyl)imidazo[1,2-a]pyrimidin-3-yl]methyl}-2,5-diazabicyclo[2.2.2]oct-2-yl)methanone, (3-fluoro-6-methoxypyridin-2-yl)(5-{[2-(4-isopropylphenyl)imidazo[1,2-a]pyrimidin-3-yl]methyl}-2,5-diazabicyclo[2.2.2]oct-2-yl)methanone, (3-fluoro-6-methoxypyridin-2-yl)(5-[2-(4-isopropylphenyl)imidazo[1,2-a]pyrimidin-3-yl]methyl}diazabicyclo[2.2.2]oct-2-yl)methanone, (5-{[2-(4-isopropylphenyl)imidazo[1,2-a]pyrimidin-3-yl]methyl}-2,5-diazabicyclo[2.2.2]oct-2-yl)(6-methoxypyridin-2-yl)methanone, (5-{[2-(4-isopropylphenyl)imidazo[1,2-a]pyrimidin-3-yl]methyl}-2,5- diazabicyclo[2.2.2]oct-2-yl)(6-methoxypyridin-2-yl)methanone,[6-(difluoromethoxy)pyridin-2-yl](5-{[2-(4-isopropylphenyl)imidazo[1,2-a]pyrimidin-3-yl]methyl}-2,5-diazabicyclo[2.2.2]oct-2- yl)methanone, (5-{[2-(4-isopropylphenyl)imidazo[1,2-a]pyrimidin-3-yl]methyl}-2,5-diazabicyclo[2.2.2]oct-2-yl)(6-methoxy-3-methylpyridin-2-yl)methanone, (5-{[2-(4-bromophenyl)imidazo[1,2-a]pyrimidin-3- yl]methyl}-2,5-diazabicyclo[2.2.2]oct-2-yl)(2-fluorophenyl)methanone, (5-{[2-(4-bromophenyl)imidazo[1,2-a]pyrimidin-3-yl]methyl}-2,5- diazabicyclo[2.2.2]oct-2-yl)(cyclopentyl)methanone, (5-{[2-(4-bromophenyl)imidazo[1,2-a]pyrimidin-3-yl]methyl}-2,5-diazabicyclo[2.2.2]oct-2-yl)(3-fluoro-6-methoxypyridin-2-yl)methanone, (3-{[2-(4-bromophenyl)imidazo[1,2-a]pyrimidin-3-yl]methyl}-3,8-diazabicyclo[3.2.1]oct-8-yl)(3-fluoro-6-methoxypyridin-2-yl)methanone, (3-{[2-(4-bromophenyl)imidazo[1,2-a]pyrimidin-3-yl]methyl}-3,8-diazabicyclo[3.2.1]oct-8-yl)(2-fluorophenyl)methanone, (3-{[2-(4-bromophenyl)imidazo[1,2-a]pyrimidin-3-yl]methyl}-3,8-diazabicyclo[3.2.1]oct-8-yl)(cyclopentyl)methanone, 3-{[2-(4-Chlorophenyl)imidazo[1,2-a]pyrimidin-3-yl]methyl}-N-(2,4- difluorophenyl)-3,8-diazabicyclo[3.2.1]octane-8-carboxamide, 3-{[2-(4-chlorophenyl)imidazo[1,2-a]pyrimidin-3-yl]methyl}-N-isopropyl-3,8-diazabicyclo[3.2.1]octane-8-carboxamide, 3-{[2-(4-Chlorophenyl)imidazo[1,2-a]pyrimidin-3-yl]methyl}-N-cyclopropyl-3,8- diazabicyclo[3.2.1]octane-8-carboxamide, 3-{[2-(4-chlorophenyl)imidazo[1,2-a]pyrimidin-3-yl]methyl}-N-(2,5-dicyhloro-4-methoxyphenyl)-3,8-diazabicyclo[3.2.1]octane-8-carboxamide, N-(3-chlorophenyl)-3-{[2-(4-chlorophenyl)imidazo[1,2-a]pyrimidin-3-yl]methyl}-3,8-diazabicyclo[3.2.1]octane-8-carboxamide, 3-{[2-(4-chlorophenyl)imidazo[1,2-a]pyrimidin-3-yl]methyl}-N-(2,6-difluorobenzyl)-3,8-diazabicyclo[3.2.1]octane-8-carboxamide, 3-{[2-(4-chlorophenyl)imidazo[1,2-a]pyrimidin-3-yl]methyl}-N-(2,6-dichlorophenyl)-3,8-diazabicyclo[3.2.1]octane-8-carboxamide, 3-{[2-(4-chlorophenyl)imidazo[1,2-a]pyrimidin-3-yl]methyl}-N-(2,6- dimethylphenyl)-3,8-diazabicyclo[3.2.1]octane-8-carboxamide, 3-{[2-(4-chlorophenyl)imidazo[1,2-a]pyrimidin-3-yl]methyl}-N-(2-fluorophenyl)-3,8-diazabicyclo[3.2.1]octane-8-carboxamide, 3-{[2-(4-chlorophenyl)imidazo[1,2-a]pyrimidin-3-yl]methyl}-N-(2,3- dichlorophenyl)-3,8-diazabicyclo[3.2.1]octane-8-carboxamide, 3-{[2-(4-chlorophenyl)imidazo[1,2-a]pyrimidin-3-yl]methyl}-N-(2-ethylphenyl)-3,8-diazabicyclo[3.2.1]octane-8-carboxamide, N-(2-chlorophenyl)-3-{[2-(4-chlorophenyl)imidazo[1,2-a]pyrimidin-3-yl]methyl}-3,8-diazabicyclo[3.2.1]octane-8-carboxamide, 3-{[2-(4-chlorophenyl)imidazo[1,2-a]pyrimidin-3-yl]methyl}-N-[2-chloro-5-(trifluoromethyl)phenyl]-3,8-diazabicyclo[3.2.1]octane-8-carboxamide, 3-{[1-(4-chlorophenyl)imidazo[1,2-a]pyrimidin-3-yl]methyl}-N-(2-ethyl-6-methylphenyl)-3,8-diazabicyclo[3.2.1]octane-8-carboxamide, 3-{[2-(4-chlorophenyl)imidazo-[1,2-a]pyrimidin-3-yl]methyl}-N-(2,5-dimethylphenyl)-3,8-diazabicyclo[3.2.1]octane-8-carboxamide, 3-{[2-(4-Chlorophenyl)imidazo[1,2-a]pyrimidin-3-yl]methyl}-N-cyclohexyl-3,8-diazabicyclo[3.2.1]octane-8-carboxamide, 3-{[2-(4-Chlorophenyl)imidazo[1,2-a]pyrimidin-3-yl]methyl}-N-isobutyl-3,8-diazabicyclo[3.2.1]octane-8-carboxamide, 3-{[2-(4-chlorophenyl)imidazo[1,2-a]pyrimidin-3-yl]methyl}-N-(3,4-dimethoxyphenyl)-3,8-diazabicyclo[3.2.1]octane-8-carboxamide, 3-{[2-(4-chlorophenyl)imidazo[1,2-a]pyrimidin-3-yl]methyl}-N-{4-[(trifluoromethyl)sulfanyl]phenyl}-3,8-diazabicyclo[3.2.1]octane-8-carboxamide, 3-{[2-(4-chlorophenyl)imidazo[1,2-a]pyrimidin-3-yl]methyl}-N-(3-fluorophenyl)-3,8-diazabicyclo[3.2.1]octane-8-carboxamide, 3-{[2-(4-chlorophenyl)imidazo[1,2-a]pyrimidin-3-yl]methyl}-N-(2,6-difluorophenyl)-3,8-diazabicyclo[3.2.1]octane-8-carboxamide, 3-{[2-(4-chlorophenyl)imidazo[1,2-a]pyrimidin-3-yl]methyl}-N-(4-chloro- 2-(trifluoromethyl)phenyl]-3,8-diazabicyclo[3.2.1]octane-8-carboxamide, 3-{[2-(4-chlorophenyl)imidazo[1,2-a]pyrimidin-3-yl]methyl}-N-(2-methylbenzyl)-3,8-diazabicyclo[3.2.1]octane-8-carboxamide, 3-{[2-(4-chlorophenyl)imidazo[1,2 -a]pyrimidin-3-yl]methyl}-N-methyl-N-phenyl-3,8-diazabicyclo[3.2.1]octane-8-carboxamide, 3-{[2-(4-chlorophenyl)imidazo[1,2-a]pyrimidin-3-yl]methyl}-N,N-diethyl-3,8-diazabicyclo[3.2.1]octane-8-carboxamide, (3-{[2-(4-chlorophenyl)imidazo[1,2-a]pyrimidin-3-yl]methyl}-3,8-diazabicyclo[3.2.1]oct-8-yl)(morpholin-4-yl)methanone, 3-{[2(4-chlorophenyl)imidazo[1,2-a]pyrimidin-3-yl]methyl}-N,N-diisopropyl-3,8- diazabicyclo[3.2.1]octane-8-carboxamide, 3-{[2-(4-chlorophenyl)imidazo[1,2-a]pyrimidin-3-yl]methyl}-N-cyclohexyl-N-ethyl-3,8-diazabicyclo[3.2.1]octane-8-carboxamide, (3-{[2-(4-chlorophenyl)imidazo[1,2-a]pyrimidin-3-yl]methyl}-3,8-diazabicyclo[3.2.1]oct-8-yl)(pyrrolidin-1-yl)methanone, 3-{[2-(4-chlorophenyl)imidazo[1,2-a]pyrimidin-3-yl]methyl}-N-ethyl-N-phenyl-3,8-diazabicyclo[3.2.1]octane-8-carboxamide, 3-{[2-(4-chlorophenyl)imidazo[1,2-a]pyrimidin-3-yl]methyl}-N-isopropyl-N-methyl-38-diazabicyclo[3.2.1]octane-8-carboxamide, (3-{[2-(4-chlorophenyl)imidazo[1,2-a]pyrimidin-3-yl]methyl}-3,8-diazabicyclo[3.2.1]oct-8-yl)(piperidin-1-yl)methanone, 3-{[2-(4-chlorophenyl)imidazo[1,2-a]pyrimidin-3-yl]methyl}-N-ethyl-N-(4- methylphenyl)-3,8-diazabicyclo[3.2.1]octane-8-carboxamide, N-(4-chlorophenyl)-3-{[2-(4-chlorophenyl)imidazo[1,2-a]pyrimidin-3-yl]methyl}-N-isopropyl-3,8-diazabicyclo[3.2.1]octane-8-carboxamide, 3-{[2-(4-chlorophenyl)imidazo[1,2-a]pyrimidin-3-yl]methyl}-N,N-dimethyl-3,8-diazabicyclo[3.2.1]octane-8-carboxamide, 3-{[2-(4-chlorophenyl)imidazo[1,2-a]pyrimidin-3-yl]methyl}-N-(4-ethoxyphenyl)-N-methyl-3,8-diazabicyclo[3.2.1]octane-8-carboxamide, 3-{[2-(4-chlorophenyl)imidazo[1,2 -a]pyrimidin-3-yl]methyl}-N-(3-methoxybenzyl)-N-methyl-3,8-diazabicyclo[3.2.1]octane-8-carboxamide, (3-{[2-(4-Chlorophenyl)imidazo[1,2-a]pyrimidin-3-yl]methyl}-3,8-diazabicyclo[3.2.1]oct-8-yl)(thiomorpholin-4-yl)methanone, methyl 3-{[2-(4-chlorophenyl)imidazo[1,2-a]pyrimidin-3-yl]methyl}-3,8-diazabicyclo[3.2.1]octane-8-carboxylate, ethyl 3-{[2-(4-chlorophenyl)imidazo[1,2-a]pyrimidin-3-yl]methyl}-3,8-diazabicyclo[3.2.1]octane-8-carboxylate, cyclopentyl-{[2-(4-chlorophenyl)imidazo[1,2-a]pyrimidin-3-yl]methyl}-3,8-diazabicyclo[3.2.1]octane-8-carboxylate, propyl 3-{[2-(4-chlorophenyl)imidazo[1,2-a]pyrimidin-3-yl]methyl}-3,8-diazabicyclo[3.2.1]octane-8-carboxylate, cyclohexylmethyl 3-{[2-(4-chlorophenyl)imidazo[1,2-a]pyrimidin-3-yl]methyl}-3,8-diazabicyclo[3.2.1 ]octane-8-carboxylate, cyclohexyl 3-{[2-(4-chlorophenyl)imidazo[1,2-a]pyrimidin-3-yl]methyl}-3,8-diazabicyclo[3.2.1]octane-8-carboxylate, 2,2-dimethylpropyl 3-{[2-(4-chlorophenyl)imidazo[1,2-a]pyrimidin-3-yl]methyl}-3,8- diazabicyclo[3.2.1]octane-8-carboxylate, tert-Butyl 3{[2-(4-isopropylphenyl)imidazo[1,2-a]pyrimidin-3-yl]methyl}-3,8-diazabicyclo[3.2.1]octane-8-carboxylate, (5-Cyclopropyl-1,3-oxazol-4-yl)(3{[2-(4-isopropylphenyl)imidazo[1,2-a]pyrimidin-3-yl]methyl}-3,8-diazabicyclo[3.2.1]oct-8-yl)methanone, tert-Butyl 3-{[2-(4-cyclopropylphenyl)imidazo[1,2-a]pyrimidin-3-yl ]methyl}-3,8-diazabicyclo[3.2.1]octane-8-carboxylate, (3-{[2-(4-cyclopropylphenyl)imidazo[1,2-a]pyrimidin-3-yl]methyl}-3,8-diazabicyclo[3.2.1]octan-8-yl)(2-fluorophenyl)methanone, cyclopentyl (3-{[2-(4-cyclopropylphenyl)imidazo[1,2-a]pyrimidin-3-yl]methyl}-3,8-diazabicyclo[3.2.1]octan-8-yl)methanone, (3-{[2-(4-cyclopropylphenyl)imidazo[1,2-a]pyrimidin-3-yl]methyl}-3,8-diazabicyclo[3.2.1]octan-8-yl)(3-fluoro-6-methoxypyridin-2-yl)methanone, (3-chloro-6-methoxypyridin-2-yl)(3-{[2-(4-cyclopropylphenyl)imidazo[1,2-a]pyrimidin-3-yl]methyl}-3,8-diazabicyclo[3.2.1]octan-8-yl)methanone, (3-{[2-(4-cyclopropylphenyl)imidazo[1,2-a]pyrimidin-3-yl]methyl}-3,8-diazabicyclo[3.2.1]octan-8-yl)(6-methoxypyridin-2-yl)methanone, (3-{[2-(4-cyclopropylphenyl)imidazo[1,2-a]pyrimidin-3-yl]methyl}-3,8-diazabicyclo[3.2.1]octan-8-yl)[6-(difluoromethoxy)pyridin-2-yl]methanone, (5-cyclopropyl-1,3- oxazol-4-yl)(3{[2-(4-cyclopropylphenyl)imidazo[1,2-a]pyrimidin-3-yl]methyl}-3,8-diazabicyclo[3.2.1]octan-8-yl)methanone, tert-Butyl 6-{[2(4-chlorophenyl)imidazo[1,2-a]pyrimidin-3-yl]methyl}-2,6-diazabicyclo[3.2.2]nonane-2-carboxylate, tert-butyl 6-{[2-(5-chloropyridin-2-yl)imidazo[1,2-a]pyridin-3-yl]methyl}-2,6-diazabicyclo[3.2.2]nonane-2-carboxylate, tert-butyl 6-{[2-(4-chlorophenyl)imidazo[1,2-a]pyridin-3-yl]methyl}-2,6-diazabicyclo[3.2.2]nonane-2-carboxylate, (−)-tert-butyl 6-{[2-(4-isopropylphenyl)imidazo[1,2-a]pyrimidin-3-yl]methyl}-2,6-diazabicyclo[3.2.2]nonane-2-carboxylate, tert-Butyl 9-{[2-(4-isopropylphenyl)imidazo[1,2-a]pyrimidin-3-yl]methyl}-3,9-diazabicyclo[4.2.1]nonane-3-carboxylate, 3-{[2-(4-Chlorophenyl)imidazo[1,2-a]pyridin-3-yl]methyl}-8-oxa-3,10-diazabicyclo[4.3.1]dec-10-yl](3-fluoro-6-methoxypyridin-2-yl)methanone, 3-{[2-(4-Chlorophenyl)imidazo[1,2-a]pyrimidin-3-yl]methyl}-8- oxa-3,10-diazabicyclo[4.3.1]dec-10-yl](3-fluoro-6-methoxypyridin-2-yl)methanone,[3-{[2(5-Chloropyridin-2-yl)imidazo[1,2-a]pyridin-3-yl]methyl}-3,9-diazabicyclo[4.2.1]non-9-yl](3-fluoro-6-methoxypyridin-2-yl)methanone, [6-{[2-(4-Chlorophenyl)imidazo[1,2-a]pyrimidin-3-yl]methyl}-2,6-diazabicyclo[3.2.2]non-2-yl](3-fluoro-6-methoxypyridin-2-yl)methanone,[6-{[2-(5-chloropyridin-2-yl)imidazo[1,2-a]pyridin-3-yl]methyl}-2,6-diazabicyclo[3.2.2]non-2-yl](6-methoxypyridin-2-yl)methanone, [6-{[2-(5-Chloropyridin-2-yl)imidazo[1,2-a]pyridin-3-yl]methyl}-2,6-diazabicyclo[3.2.2]non-2-yl](3-fluoro-6-methoxypyridin-2-yl)methanone, (3-Chloro-6-methoxypyridin-2-yl)[6-{[2-(5-chloropyridin-2-yl)imidazo[1,2-a]pyridin-3-yl]methyl}-2,6-diazabicyclo[3.2.2]non-2-yl]methanone, [6-{[2-(4-Chlorophenyl)imidazo[1,2-a]pyrimidin-3-yl]methyl}-2,6-diazabicyclo[3.2.2]non-2-yl](6-methoxypyridin-2-yl)methanone[6-{[2-(4-Chlorophenyl)imidazo[1,2-a]pyrimidin-3-yl]methyl}-2,6-diazabicyclo[3.2.2]non-2-yl](2-fluorophenyl)methanone, (3-chloro-6-methoxypyridin-2-yl)[6-{[1-(4-chlorophenyl)imidazo[1,2-a]pyrimidin-3-yl]methyl}-2,6-diazabicyclo[3.2.2]non-2-yl]methanone, (4-amino-1,2-oxadiazol-3-yl)[6-{[2-(4-chlorophenyl)imidazo[1,2-a]pyrimidin-3-yl]methyl}-2,6-diazabicyclo[3.2.2]non-2-yl]methanone, [6-{[2-(4-Chlorophenyl)imidazo[1,2-a]pyridin-3-yl]methyl}-2,6-diazabicyclo[3.2.2]non-2-yl](2-fluorophenyl)methanone, [6-{[1-(4- chlorophenyl)imidazo[1,2-a]pyridin-3-yl]methyl}-2,6-diazabicyclo[3.2.2]non-2-yl](6-methoxypyridin-2-yl)methanone,[6-{[2-(4-chlorophenyl)imidazo[1,2-a]pyridin-3-yl]methyl}-2,6-diazabicyclo[3.2.2]non-2-yl](3-fluoro-6-methoxypyridin-2-yl)methanone, (3-chloro-6-methoxypyridin-2-yl)[6-{[2-(4-chlorophenyl)imidazo[1,2-a]pyridin-3-yl]methyl}-2,6-diazabicyclo[3.2.2]non-2-yl]methanone, (4-amino-1,2,5-oxadiazol-3-yl)[6-{[2-(4-chlorophenyl)imidazo[1,2-a]pyridin-3-yl]methyl}-2,6-diazabicyclo[3.2.2]non-2-yl]methanone, [6-{[2-(5-Chloropyridin-2-yl)imidazo[1,2-a] pyridin-3-yl]methyl}-2,6-diazabicyclo[3.2.2]non-2-yl](2-fluorophenyl)methanone, [6-{[2(4- Chlorophenyl)imidazo[1,2-a]pyrimidin-3-yl]methyl}-2,6-diazabicyclo[3.2.2]non-2-yl](2-fluorophenyl)methanone, [6-{[2-(4-Chlorophenyl)imidazo[1,2-a]pyrimidin-3-yl]methyl}-2,6-diazabicyclo[3.2.2]non-2-yl](6-methoxypyridin-2-yl)methanone, [6-{[2-(4-Chlorophenyl)imidazo[1,2-a]pyrimidin-3-yl]methyl}-2,6-diazabicyclo[3.2.2]non-2-yl](3-fluoro-6-methoxypyridin-2-yl)methanone, (3-chloro-6-methoxypyridin-2-yl)[6-{[2-(4-chlorophenyl)imidazo[1,2-a]pyrimidin-3-yl]methyl}-2,6-diazabicyclo[3.2.2]non-2-yl]methanone, (4-amino-1,2,5 -oxadiazol-3-yl)[6-{[2-(4-chlorophenyl)imidazo[1,2-a]pyrimidin-3-yl]methyl}-2,6-diazabicyclo[3.2.1]non-2-yl]methanone, [6-{[2(4-Chlorophenyl)imidazo[1,2-a]pyridin-3-yl]methyl}-2,6-diazabicyclo[3.2.2]non-2-yl](2-fluorophenyl)methanone, [6-{[2-(4-chlorophenyl)imidazo[1,2-a]pyridin-3-yl]methyl}-2,6-diazabicyclo[3.2.2]non-2-yl](6-methoxypyridin-2-yl)methanone, [6-{[1-(4-chlorophenyl)imidazo[1,2-a]pyridin-3yl]methyl}-2,6-diazabicyclo[3.2.2]non-2-yl](3-fluoro-6-methoxypyridin-2-yl)methanone, (3-chloro-6-methoxypyridin-2-yl)[6-{[2-(4-chlorophenyl)imidazo[1,2-a]pyridin-3-yl]methyl}-2,6-diazabicyclo[3.2.2]non-2-yl]methanone, (4-amino-1,2,5-oxadiazol-3-yl)[6-{[1-(4-chlorophenyl)imidazo[1,2-a]pyridin-3-yl]methyl-2,6-diazabicyclo[3.2.2]non-2-yl]methanone, [6-{[2-(4 -isopropylphenyl)imidazo[1,2-a]pyrimidin-3-yl]methyl}-2,6-diazabicyclo[3.2.1]non-2-yl](6-methoxypyridin-2-yl)methanone, (3-fluoro-6-methoxypyridin-2-yl)[6-{[2-(4-isopropylphenyl)imidazo[1,2-a]pyrimidin-3-yl]methyl}-2,6-diazabicyclo[3.2.2]non-2-yl]methanone, (3-chloro-6-methoxypyridin-2-yl)[6-{[2-(4-isopropylphenyl)imidazo[1,2-a]pyrimidin-3-yl]methyl}-2,6-diazabicyclo[3.2.2]non-2-yl]methanone, cyclopentyl[6-{[2-(4-isopropylphenyl)imidazo[1,2-a]pyrimidin-3-yl]methyl}-2,6-diazabicyclo[3.2.2]non-2-yl]methanone,[6-(difluoromethoxy)pyridin-2-) yl][6-{[1-(4-isopropylphenyl)imidazo[1,2-a]pyrimidin-3-yl]methyl}-2,6-diazabicyclo[3.2.2]non-2-yl]methanone, (2-fluorophenyl)[6-{[2-(4-isopropylphenyl)imidazo[1,2-a]pyrimidin-3-yl]methyl}-2,6-diazabicyclo[3.2.2]non-2-yl]methanone, (2-fluorophenyl)[6-{[1-(4-isopropylphenyl)imidazo[1,2-a]pyrimidin-3-yl]methyl}-2,6-diazabicyclo[3.2.2]non-2-yl]methanone,[6-{[2-(4-isopropylphenyl)imidazo[1,2 -a]pyrimidin-3-yl]methyl}-2,6- diazabicyclo[3.2.2]non-2-yl](6-methoxypyridin-2-yl)methanone, (3-fluoro-6-methoxypyridin-2-yl)[6-{[2-(4-isopropylphenyl)imidazo[1,2-a]pyrimidin-3-yl]methyl}-2,6-diazabicyclo[3.2.2]non-2- yl]methanone, (3-chloro-6-methoxypyridin-2-yl)[6-{[2-(4-isopropylphenyl)imidazo[1,2-a]pyrimidin-3-yl]methyl}-2,6-diazabicyclo[3.2.2]non-2-yl]methanone, [6-(difluoromethoxy)pyridin-2-yl][6-{[2-(4-isopropylphenyl)imidazo[1,2-a]pyrimidin-3-yl]methyl}-2,6-diazabicyclo[3.2.2 ]non-2-yl]methanone, cyclopentyl[6-{[1-(4-isopropylphenyl)imidazo[1,2-a]pyrimidin-3-yl]methyl}-2,6-diazabicyclo[3.2.2]non-2-yl]methanone, (3-fluoro-6-methoxypyridin-2-yl)[9-[2-(4-isopropylphenyl)imidazo[1,2-a]pyrimidin-3-yl]methyl}-3,9-diazabicyclo[4.2.1]non-3-yl]methanone, (3-chloro-6-methoxypyridin-2-yl)[9-{[2-(4-isopropylphenyl)imidazo[1,2-a]pyrimidin-3-yl]methyl}-3,9-diazabicyclo[4.2.1]non-3-yl]methanone,[9-{[2-(4-isopropylphenyl)imidazo[1,2-a]pyrimidin-3-yl]methyl}-3,9-diazabicyclo[4.2.1]non-3-yl](6-methoxypyridin-2-yl)methanone, [6-(difluoromethoxy)pyridin-2-yl][9-{[2-(4-isopropylphenyl)imidazo[1,2-a]pyrimidin-3-yl]methyl}-3,9-diazabicyclo[4.2.1]non-3-yl]methanone, cyclopentyl[9-{[2-(4-isopropylphenyl)imidazo[1,2-a]pyrimidin-3-yl]methyl}-3,9-diazabicyclo[4.2.1]non-3-yl]methanone, (3-fluoro-6-methoxypyridin-2-yl)[9-{[2-(4-isopropylphenyl)imidazo[1,2-a]pyrimidin-3-yl]methyl}-3,9-diazabicyclo yl]methanone (3-chloro-6-methoxypyridin-2-yl)[9-{[2-(4-isopropylphenyl)imidazo[1,2-a]yl]methyl}-3,9-diazabicyclo[4.2.1]non-3-yl]methanone, [9-{[2-(4-isopropylphenyl)imidazo[1,2-a]pyrimidin-3-yl]methyl}-3,9-diazabicyclo[4.2.1]non-3-yl](6-methoxypyridin-2-yl)methanone, [6-(difluoromethoxy)pyridin-2-yl][9-{[2-(4- isopropylphenyl)imidazo[1,2-a]pyrimidin-3-yl]methyl}-3,9-diazabicyclo[4.2.1]non-3-yl]methanone, cyclopentyl[9-{[2-(4-isopropylphenyl)imidazo[1,2-a]pyrimidin-3-yl]methyl}-3,9-diazabicyclo[4.2.1]non-3-yl]methanone, (−)-(2-fluorophenyl)[9-{[2-(4-isopropylphenyl)imidazo[1,2-a]pyrimidin-3-yl]methyl}-3,9-diazabicyclo[4.2.1]non-3-yl]methanone, [6-{[2-(4-chlorophenyl)imidazo[1,2-a]pyrimidin-3-yl]methyl}-2,6-diazabicyclo[4.2.1non-2-yl][6-(trifluoromethoxy)pyridin-2-yl]methanone, [6-{[1-(4-chlorophenyl)imidazo[1,2-a]pyrimidin-3-yl]methyl}-2,6-diazabicyclo[3.2.2]non-2-yl][6-(difluoromethoxy)pyridin-2-yl]methanone, [3-{[2-(4-chlorophenyl)imidazo[1,2-a]pyridin-3-yl]methyl}-3,9-diazabicyclo[4.2.1]non-9-yl](2-fluorophenyl)methanone, [3-{[2-(4-chlorophenyl)imidazo[1,2-a]pyridin-3-yl]methyl}-3,9-diazabicyclo[4.2.1]non-9-yl](6-methoxypyridin-2-yl)methanone, (3-{[2-(4-chlorophenyl)imidazo[1,2-a]pyridin-3-yl]methyl}-8-oxa-3,10-diazabicyclo[4.3.1]dec-10-yl](2-fluorophenyl)methanone, [3-{[2-(4-chlorophenyl)imidazo[1,2-a]pyridin-3-yl]methyl}-8-oxa-3,10-diazabicyclo[4.3.1]dec-10-yl](6-methoxypyridin-2-yl)methanone, [3-{[2-(4-chlorophenyl)imidazo[1,2-a]pyridin-3-yl]methyl}-8-oxa-3,10-diazabicyclo[4.3.1]dec-10-yl](4-methyl-1,2,5-oxadiazol-3-yl)methanone, [3-{[2-(4-chlorophenyl)imidazo[1,2-a]pyrimidin-3-yl]methyl}-8- oxa-3,10-diazabicyclo[4.3.1]dec-10-yl](2-fluorophenyl)methanone, [3-{[2-(4-chlorophenyl)imidazo-[1,2-a]pyrimidin-3-yl]methyl}-8-oxa-3,10- diazabicyclo[4.3.1]dec-10-yl](6-methoxypyridin-2-yl)methanone, [3{[2-(4-chlorophenyl)imidazo[1,2-a]pyrimidin-3-yl]methyl}-8-oxa-3,10-diazabicyclo[4.3.1]dec-10-yl](4-methyl-1,2,5-oxadiazol-3-yl)methanone, (4-amino-1,2,5-oxadiazol-3-yl)[3-{[2-4-chlorophenyl)imidazo[1,2-a]pyrimidin-3-yl]methyl}-8-oxa-3,10-diazabicyclo[4.3.1]dec-10-yl]methanone, [3-{[2-(4-chlorophenyl)imidazo[1,2-a]pyridin-3-yl]methyl}-3,9-diazabicyclo[4.2.1]non-9-yl](3-fluoro-6-methoxypyridin-2-yl)methanone, [3-{[2-(4-chlorophenyl)imidazo[1,2-a]pyrimidin-3-yl]methyl}-3,9-diazabicyclo[4.2.1]non-9-yl](2-fluorophenyl)methanone, [3-{[2-(4-chlorophenyl)imidazo[1,2-a]pyrimidin-3-yl]methyl}-3,9-diazabicyclo[4.2.1]non-9-yl](3-fluoro-6-methoxypyridin-2-yl)methanone, [3-{[2-(5-chloropyridin-2-yl)imidazo[1,2-a]pyridin-3-yl]methyl}-3,9- diazabicyclo[4.2.1]non-9-yl](6-methoxypyridin-2-yl)methanone, [3-{[2(4-chlorophenyl)imidazo[1,2-a]pyrimidin-3-yl]methyl}-3,9-diazabicyclo[4.2.1]non-9-yl](6-methoxypyridin-2-yl)methanone, [3-{[2-(4-Chlorophenyl)imidazo[1,2-a]pyridin-3-yl]methyl}-3,9-diazabicyclo[4.2.1]non-9-yl](2-fluorophenyl)methanone, [3-{[2-(4-Chlorophenyl)imidazo[1,2-a]pyridin-3-yl]methyl}-3,9-diazabicyclo[4.2.1]non-9-yl](6-methoxypyridin-2-yl)methanone, [3-{[2-(4-chlorophenyl)imidazo[1,2-a]pyridin-3-yl]methyl}-8-oxa-3,10-diazabicyclo[4.3.1]dec-10-yl](3-fluoro-6-methoxypyridin-2-yl)methanone, [3-{[2-(4-chlorophenyl)imidazo[1,2-a]pyrimidin-3-yl]methyl}-8-oxa-3,10- diazabicyclo[4.3.1]dec-10-yl](3-fluoro-6-methoxypyridin-2-yl)methanone, [3-{[2-(4-chlorophenyl)imidazo[1,2-a]pyridin-3-yl]methyl}-3,6-diazabicyclo[3.2.2]non-6-yl](3-fluoro-6-methoxypyridin-2-yl)methanone, [3-{[2-(4-chlorophenyl)imidazo[1,2-a]pyridin-3-yl]methyl}-3,6-diazabicyclo[3.2.2]non-6-yl](6-methoxypyridin-2-yl)methanone, [3-{[2-(5-chloropyridin-2-yl)imidazo[1,2-a]pyridin-3-yl]methyl}-3,6-diazabicyclo[3.2.2]non-6-yl](3-fluoro-6-methoxypyridin-2-yl)methanone, [3-{[2-(4-chlorophenyl)imidazo[1,2-a]pyrimidin-3-yl]methyl}-3,6-diazabicyclo[3.2.2]non-6-yl](3-fluoro-6-methoxypyridin-2-yl)methanone, [3-{[2-(4-chlorophenyl)imidazo[1,2-a]pyrimidin-3-yl]methyl}-3,6-diazabicyclo[3.2.2]non-6-yl](6-methoxypyridin-2-yl)methanone, (3-chloro-6-methoxypyridin-2-yl)(3-{[2-(4-chlorophenyl)imidazo[1,2-a]pyrimidin-3-yl]methyl}-3,9-diazabicyclo[4.2.1]non-9-yl)methanone, (3-chloro-6-methoxypyridin-2-yl)(3-{[2-(5-chloropyridin-2-yl)imidazo[1,2-a]pyridin-3-yl]methyl}-3,9-diazabicyclo[4.2.1]non-9-yl)methanone, (3-Chloro-6-methoxypyridin-2-yl)[3-{[2-(4-isopropylphenyl)imidazo[1,2-a]pyrimidin-3-yl]methyl}-3,9-diazabicyclo[4.2.1]nonan-9-yl]methanone, (3-Fluoro-6-methoxypyridin-2-yl)[3-{[2-(4-isopropylphenyl)imidazo[1,2-a]pyrimidin-3-yl]methyl}-3,9-diazabicyclo[4.2.1]nonan-9-yl]methanone, [3-{[2-(4-Isopropylphenyl)imidazo[1,2-a]pyrimidin-3-yl]methyl}-3,9-diazabicyclo[4.2.1]nonan-9-yl](6-methoxypyridin-2-yl)methanone, (3-Chloro-6-methoxypyridin-2-yl)[3-{[2(4-isopropylphenyl)imidazo[1,2-a]pyrimidin-3-yl]methyl}-3,9-diazabicyclo[4.2.1]nonan-9-yl]methanone.

In a more preferred embodiment of the present invention is directed to combinations of compounds of formula (I) which arc selected from the group consisting of:

(S)-1-((2,3-Dihydrobenzo[b][1,4]dioxin-2-yl)methyl)-4-(2-(methoxymethyl)phenyl)piperazine, (R)-1-((2,3-Dihydrobenzo[b][1,4]dioxin-2-yl)methyl)-4-(2-(methoxymethyl)phenyl)piperazine, (2-(4-(2,3-Dihydrobenzo[b][1,4]dioxin-2-yl)methyl)piperazin-1-yl)-6-fluorophenyl)methanol, (S)-1-((2,3-Dihydrobenzo[b][1,4]dioxin-2-yl)methyl)-4-(2-(furan-2-yl)phenyl)piperazine, (S)-1-((2,3-Dihydrobenzo[b][1,4]dioxin-2-yl)methyl)-4-o-tolylpiperazine, methyl 2-(4-((2,3-dihydrobenzo[b][1,4]dioxin-2-yl)methyl)-1,4-diazepan-1-yl)benzoate, (S)-4(2,3-dihydrobenzo[b][1,4]dioxin-2-yl)methyl)-4-(3-(methoxymethyl)pyridin-2-yl)piperazine and of compounds of formula (II) which are selected from the group consisting of:

(4-{[2-(4-Bromophenyl)imidazo[1,2-a]pyridin-3-yl]methyl}piperazin-1-yl)(cyclopentyl)methanone, (4-{[2-(4-Chlorophenyl)imidazo[1,2-a]pyridin-3-yl]methyl}piperazin-1-yl)(cyclopentyl)methanone, (4-{[2-(4-Chlorophenyl)imidazo[1,2-a]pyridin-3-yl]methyl}piperazin-1-yl)(6-methoxypyridin-2-yl)methanone, (4-{[2-(4-Bromophenyl)imidazo[1,2-a]pyridin-3-yl]methyl}piperazin-1-yl)(2-fluorophenyl)methanone, (4-{[2-(4-chlorophenyl)imidazo[1,2-a]pyridin-3-yl]methyl}piperazin-1-yl)(6- isopropoxypyridin-2-yl)methanone, (4-{[2(4-bromophenyl)imidazo[1,2-a]pyridin-3-yl]methyl}piperazin-1-yl)(6-methoxypyridin-2-yl)methanone, (4-{[2-(4-Chlorophenyl)imidazo[1,2-a]pyridin-3-yl]methyl}piperazin-1-yl)[6-(trifluoromethoxy)pyridin-2-yl]methanone, (4-{[2-(4-Chlorophenyl)imidazo[1,2-a]pyridin-3-yl]methyl}piperazin-1-yl)(3-fluoro-6-methoxypyridin-2-yl)methanone, [5-{[2-(4-Chlorophenyl)imidazo[1,2-a]pyridin-3-yl]methyl}hexahydropyrrolo[3,4-c]pyrrol-2(1H)-yl](6-methoxypyridin-2-yl)methanone, [5-{[2-(4 -Isopropylphenyl)imidazo[1,2-a]pyridin-3-yl]methyl}hexahydropyrrolo[3,4-c]pyrrol-2(1H)-yl](6-methoxypyridin-2-yl)methanone, (3-Fluoro-6-methoxypyridin-2-yl)[5-{[2-(4-isopropylphenyl)imidazo[1,2-a]pyridin-3-yl]methyl}hexahydropyrrolo[3,4-c]pyrrol-2(1H)-yl]methanone, [5-{[2-(4-Chlorophenyl)imidazo[1,2-a]pyridin-3-yl]methyl}hexahydropyrrolo[3,4-c]pyrrol-2(1H)-yl](6-methoxy-3-methylpyridin-2-yl)methanone, (−)-[(1S,4S)-5-{[2-(4- Chlorophenyl)imidazo[1,2-a]pyridin-3-yl]methyl}-2,5-diazabicyclo[2.2.2]oct-2-yl](6-methoxypyridin-2-yl)methanone, (−)-(3-Chloro-6-methoxypyridin-2-yl)[(1S,4S)-5-{[2-(4-chlorophenyl)imidazo[1,2-a]pyridin-3-yl]methyl}-2,5-diazabicyclo[2.2.2]oct-2-yl]methanone, (−)-[(1S,4S)-5-{[2-(4-Chlorophenyl)imidazol[1,2-a]pyridin-3-yl]methyl}-2,5-diazabicyclo[2.2.2]oct-2-yl](3-fluoro-6-methoxypyridin-2-yl)methanone, (5-{[2-(5-Chloropyridin-2-yl)imidazo[1,2-a]pyridin-3-yl]methyl}-2,5- diazabicyclo[2.2.2]oct-2-yl)(3-fluoro-6-methoxypyridin-2-yl)methanone, (3-Chloro-6-methoxypyridin-2-yl)(5-{[2-(5-chloropyridin-2-yl)imidazo[1,2-a]pyridin-3-yl]methyl}-2,5-diazabicyclo[2.2.2]oct-2-yl)methanone, (−)-(5-{[2-(5-Chloropyridin-2-yl)imidazo[1,2-a]pyridin-3-yl]methyl}-2,5-diazabicyclo[2.2.2]oct-2-yl)(6-methoxypyridin-2-yl)methanone, (5-{[2-(5-Chloropyridin-2-yl)imidazo[1,2-a]pyridin-3-yl]methyl}-2,5-diazabicyclo[2.2]oct-2-yl)[6-(difluoromethoxy)pyridin-2-yl]methanone, (3-{[2-(4- Chlorophenyl)imidazo[1,2-a]pyrimidin-3-yl]methyl}-3,8-diazabicyclo[3.2.1]oct-8-yl)(6-methoxypyridin-2-yl)methanone, (3-Chloro-6-methoxypyridin-2-yl)(3-{[2-(4-chlorophenyl)imidazo[1,2-a]pyrimidin-3-yl]methyl}-3,8-diazabicyclo[3.2.1]oct-8-yl)methanone, (3-{[2(4-Chlorophenyl)imidazo[1,2-a]pyrimidin-3-yl]methyl}-3,8-diazabicyclo[3.2.1]oct-8-yl)(3-fluoro-6-methoxypyridin-2-yl)methanone, (3-Chloro-6-methoxypyridin-2-yl)(5-{[2-(4-isopropylphenyl)imidazo[1,2-a]pyrimidin-3-yl]methyl}-2,5-diazabicyclo[2.2.2]oct-2-yl)methanone, (3-Fluoro-6-methoxypyridin-2-yl)(3-{[2-(4-isopropylphenyl)imidazo[1,2-a]pyrimidin-3-yl]methyl}-3,8-diazabicyclo[3.2.1]oct-8-yl)methanone, (3-Chloro-6-methoxypyridin-2-yl)(3-{[2-(4-isopropylphenyl)imidazo[1,2-a]pyrimidin-3-yl]methyl}-3,8-diazabicyclo[3.2.1]oct-8-yl)methanone, (3-{[2-(4-cyclopropylphenyl)imidazo[1,2-a]pyrimidin-3-yl]methyl}-3,8-diazabicyclo[3.2.1]octan-8-yl)(3-fluoro-6-methoxypyridin-2-yl)methanone, (3-chloro-6-methoxypyridin-2-yl)(3-{[2-(4-cyclopropylphenyl)imidazo[1,2-a]pyrimidin-3-yl]methyl}-3,8-diazabicyclo[3.2.1]octan-8-yl)methanone, 3-{[2-(4-Chlorophenyl)imidazo[1,2-a]pyridin-3-yl]methyl}-8-oxa-3,10-diazabicyclo[4.3.1]dec-10-yl](3-fluoro-6-methoxypyridin-2-yl)methanone, 3-{[2-(4-Chlorophenyl)imidazo[1,2-a]pyrimidin-3-yl]methyl}-8-oxa-3,10-diazabicyclo[4.3.1]dec-10-yl](3-fluoro-6-methoxypyridin-2-yl)methanone, [3-{[2-(5-Chloropyridin-2-yl)imidazo[1,2-a]pyridin-3-yl]methyl}-3,9-diazabicyclo[4.2.1]non-9-yl](3-fluoro-6-methoxypyridin-2-yl)methanone, (3-Fluoro-6-methoxypyridin-2-yl)[3-{[2-(4-isopropylphenyl)imidazo[1,2-a]pyrimidin-3-yl]methyl}-3,9-diazabicyclo[4.2.1 ]nonan-9-yl]methanone.

In a most preferred embodiment the present invention is directed to combinations of compounds of formula (1) which are selected from the group consisting of:

(S)-1-((2,3-dihydrobenzo[b][1,4]dioxin-2- yl)methyl)-4-(3-(methoxymethyl)pyridin-2-yl)piperazine or (S)-1-((2,3-dihydrobenzo[b][1,4]dioxin-2-yl)methyl)-4-(3-([¹¹C]-methoxymethyl)pyridin-2-yl)piperazine

and

of compounds of formula (11) which are selected from the group consisting of:

(4-{[2-(4-Bromophenyl)imidazo[1,2-a]pyridin-3-yl]methyl}piperazin-1-yl)(cyclopentyl)methanone, (4-{[2-(4-Chlorophenyl)imidazo[1,2-a]pyridin-3-yl]methyl}piperazin-1-yl)(cyclopentyl)methanone, (4-{[2-(4-Chlorophenyl)imidazo[1,2-a]pyridin-3-yl]methyl}piperazin-1-yl)(6-methoxypyridin-2-yl)methanone, (4-{[2-(4-{[2-Bromophenyl)imidazo[1,2-a]pyridin-3-yl]methyl}piperazin-1-yl)(2-fluorophenyl)methanone, (4-{[2(4-chlorophenyl)imidazo[1,2-a]pyridin-3-yl]methyl}piperazin-1-yl)(6- isopropoxypyridin-2-yl)methanone, (4-{[2-(4-bromophenyl)imidazo[1,2-a]pyridin-3-yl]methyl}piperazin-1-yl)(6-methoxypyridin-2-yl)methanone, (4-{[2-(4-Chlorophenyl)imidazo[1,2-a]pyridin-3-yl]methyl}piperazin-1-yl)[6-(trifluoromethoxy)pyridin-2-yl]methanone, (4-{[2-(4-Chlorophenyl)imidazo[1,2-a]pyridin-3-yl]methyl}piperazin-1-yl)(3-fluoro-6-methoxypyridin-2-yl)methanone, [5-{[1-(4-Chlorophenyl)imidazo[1,2-a]pyridin-3-yl]methyl}hexahydropyrrolo[3,4-c]pyrrol-2(1H)-yl](6-methoxypyridin-2-yl)methanone, [5-{[2-(4-Isopropylphenyl)imidazo[1,2-a]pyridin-3-yl]methyl}hexahydropyrrolo[3,4-c]pyrrol-2(1H)-yl](6-methoxypyridin-2-yl)methanone, (3-Fluoro-6-methoxypyridin-2-yl)[5-{[2-(4-isopropylphenyl)imidazo[1,2-a]pyridin-3-yl]methyl}hexahydropyrrolo[3,4-c]pyrrol-2(1H)-yl]methanone, [5-{[2-(4-Chlorophenyl)imidazo[1,2-a]pyridin-3-yl]methyl}hexahydropyrrolo[3,4-c]pyrrol-2(1H)-yl](6-methoxy-3-methylpyridin-2-yl)methanone, (−)-[(1S,4S)-5-{[2-(4- Chlorophenyl)imidazo[1,2-a]pyridin-3-yl]methyl}-2,5-diazabicyclo[2.2.2]oct-2-yl](6-methoxypyridin-2-yl)methanone, (−)-(3-Chloro-6-methoxypyridin-2-yl)](1S,4S)-5-{[2-(4-chlorophenyl)imidazo[1,2-a]pyridin-3-yl]methyl}-[2,5-diazabicyclo[2.2.2]oct-2-yl]methanone, (−)-[(1S,4S)-5-[2-(4-Chlorophenyl)imidazo[1,2-a]pyridin-3-yl]methyl}-2,5-diazabicyclo[2.2.2 ]oct-2-yl](3-fluoro-6-methoxypyridin-2-yl)methanone, (5-{[2-(5-Chloropyridin-2-yl)imidazo[1,2-a]pyridin-3-yl]methyl}-2,5- diazabicyclo[2.2.2]oct-2-yl)(3-fluoro-6-methoxypyridin-2-yl)methanone, (3-Chloro-6-methoxypyridin-2-yl)(5-{[2-(5-chloropyridin-2-yl)imidazo[1,2-]pyridin-3-yl]methyl}-2,5-diazabicyclo[2.2.2]oct-2-yl)methanone, (−)-(5-{[2-(5-Chloropyridin-2-yl)imidazo[1,2-a]pyridin-3-yl]methyl}-2,5-diazabicyclo[2.2.2]oct-2-yl)(6-methoxypyridin-2-yl)methanone, (5-{[2-(5-Chloropyridin-2-yl)imidazo[1,2-a]pyridin-3-yl]methyl}-2,5-diazabicyclo[2.2.2]oct-2-yl)[6-(difluoromethoxy)pyridin-2-yl]methanone, (3-{[2-(4- Chlorophenyl)imidazo[1,2-a]pyrimidin-3-yl]methyl}-3,8-diazabicyclo[3.2.1]oct-8-yl)(6-methoxypyridin-2-yl)methanone, (3-Chloro-6-methoxypyridin-2-yl)(3-{[2-(4-chlorophenyl)imidazo[1,2-a]pyrimidin-3-yl]methyl}-3,8-diazabicyclo[2.2.2]oct-8-yl)methanone, (3-{[2-(4-Chlorophenyl)imidazo[1,2 -a]pyrimidin-3-yl]methyl}-3,8-diazabicyclo[3.2.1]oct-8-yl)(3-fluoro-6-methoxypyridin-2-yl)methanone, (3 -Chloro-6-methoxypyridin-2-yl)(5-{[2-(4-isopropylphenyl)imidazo[1,2-a]pyrimidin-3-yl]methyl}-2,5-diazabicyclo[2.2.2]oct-2-yl)methanone, (3-Fluoro-6-methoxypyridin-2-yl)(3-{[2-(4-isopropylphenyl)imidazo[1,2-a]pyrimidin-3-yl]methyl}-3,8-diazabicyclo[3.2.1]oct-8-yl)methanone, (3-Chloro-6-methoxypyridin-2-yl)(3-{[2-(4-isopropylphenyl)imidazo[1,2-a]pyrimidin-3-yl]methyl}-3,8-diazabicyclo[3.2.1]oct-8-yl)methanone, (3-{[2-(4-cyclopropylphenyl)imidazo[1,2-a]pyrimidin-3-yl]methyl}-3,8-diazabicyclo[3.2.1]octan-8-yl)(3-fluoro-6-methoxypyridin-2-yl)methanone, (3-chloro-6-methoxypyridin-2-yl)(3-{[2-(4-cyclopropylphenyl)imidazo[1,2-a]pyrimidin-3-yl]methyl}-3,8-diazabicyclo[3.2.1]octan-8-yl)methanone, 3-{[2-(4-Chlorophenyl)imidazo[1,2-a]pyridin-3-yl]methyl}-8-oxa-3,10-diazabicyclo[4.3.1]dec-10-yl](3-fluoro-6-methoxypyridin-2-yl)methanone, 3-{[2-(4-Chlorophenyl)imidazo[1,2-a]pyrimidin-3-yl]methyl}-8-oxa-3,10-diazabicyclo[4.3.1]dec-10-yl](3-fluoro-6-methoxypyridin-2-yl)methanone, [3-{[2-(5-Chloropyridin-2-yl)imidazo[1,2a]pyridin-3-yl]methyl}-3,9-diazabicyclo[4.2.1]non-9-yl](3-fluoro-6-methoxypyridin-2-yl)methanone, (3-Fluoro-6-methoxypyridin-2-yl)[3-{[2-(4-isopropylphenyl)imidazo[1,2-a]pyrimidin-3-yl]methyl}-3,9-diazabicyclo[4.2.1]nonan-9-yl]methanone.

An another embodiment the present invention is directed to combinations of compounds of

(S)-1-((2,3-dihydrobenzo[b][1,4]dioxin-2-yl)methyl)-4-(3-(methoxymethyl)pyridin-2-yl)piperazine and and one or more compounds selected from the list consisting of (4-{[2-(4-Bromophenyl)imidazo[1,2-a]pyridin-3-yl]methyl}piperazin-1-yl)(cyclopentyl)methanone, (4-{[2-(4-Chlorophenyl)imidazo[1,2-a]pyridin-3-yl]methyl}piperazin-1-yl)(cyclopentyl)methanone, (4-{[2-(4-Chlorophenyl)imidazo[1,2-a]pyridin-3-yl]methyl}piperazin-1-yl)(6-methoxypyridin-2-yl)methanone, (4-{[2-(4-Bromophenyl)imidazo[1,2-a]pyridin-3-yl]methyl}piperazin-1-yl)(2- fluorophenyl)methanone, (4-{[2-(4-chlorophenyl)imidazo[1,2-a]pyridin-3-yl]methyl}piperazin-1-yl)(6-isopropoxypyridin-2-yl)methanone, (4-{[2-(4-bromophenyl)imidazo[1,2-a]pyridin-3-yl]methyl}piperazin-1-yl)(6-methoxypyridin-2-yl)methanone, (4-{[2-(4-Chlorophenyl)imidazo[1,2-a]pyridin-3-yl]methyl}piperazin-1-yl)[6-(trifluoromethoxy)pyridin-2-yl]methanone, (4-{[2-(4-Chlorophenyl)imidazo[1,2a]pyridin-3-yl]methyl}piperazin-1-yl)(3-fluoro-6-methoxypyridin-2-yl)methanone,

[5-{[2-(4-Chlorophenyl)imidazo[1,2-a]pyridin-3-yl]methyl}hexahydropyrrolo[3,4-c]pyrrol-2(1H)-yl](6-methoxypyridin-2-yl)methanone, [5-{[2-(4-Isopropylphenyl)imidazo[1,2-a]pyridin-3-yl]methyl}hexahydropyrrolo[3,4-c]pyrrol-2(1H)-yl](6-methoxypyridin-2-yl)methanone, (3-Fluoro-6-methoxypyridin-2-yl)[5{[2-(4-isopropylphenyl)imidazo[1,2-a]pyridin-3-yl]methyl}hexahydropyrrolo[3,4-c]pyrrol-2(methanone, [5-{[2-(4-Chlorophenyl)imidazo[1,2-a]pyridin-3-yl]methyl}hexahydropyrrolo[3,4-c]pyrrol-2(1H)-yl](6-methoxy-3-methylpyridin-2-yl)methanone, (−)-[(1S, 4S)-5-{[2-(4- Chlorophenyl)imidazo[1,2-a]pyridin-3-yl]methyl}-2,5-diazabicyclo[2.2.2]oct-2-yl](6-methoxypyridin-2-yl)methanone, (−)-(3-Chloro-6-methoxypyridin-2-yl)[(1S,4S)-5-{[2-(4-chlorophenyl)imidazo[1,2-a]pyridin-3-yl]methyl}-2,5-diazabicyclo[2.2.2]oct-2-yl]methanone, (−)-[(1S,4S)-5-{[2-(4-Chlorophenyl)imidazo[1,2-a]pyridin-3-yl]methyl}-2,5-diazabicyclo[2.2.2]oct-2-yl](3-fluoro-6-methoxypyridin-2-yl)methanone, (5-{[2-(5-Chloropyridin-2-yl)imidazo[1,2-a]pyridin-3-yl]methyl}-2,5 - diazabicyclo[2.2.2]oct-2-yl)(3-fluoro-6-methoxypyridin-2-yl)methanone, (3-Chloro-6-methoxypyridin-2-yl)(5-{[2-(5-chloropyridin-2-yl)imidazo[1,2-a]pyridin-3-yl]methyl}-2,5-diazabicyclo[2.2.2]oct-2-yl)methanone, (−)-(5-{[2-(5-Chloropyridin-2-yl)imidazo[1,2-a]pyridin-3-yl]methyl}-2,5-diazabicyclo[2.2.2]oct-2-yl)(6-methoxypyridin-2-yl)methanone, (5-{[2-(5-Chloropyridin-2-yl)imidazo[1,2-a]pyridin-3-yl]methyl}-2,5-diazabicyclo[2.2.2]oct-2-yl)[6-(difluoromethoxy)pyridin-2-yl]methanone, (3-{[2-(4- Chlorophenyl)imidazo[1,2-a]pyrimidin-3-yl]methyl}-3,8-diazabicyclo[3.2.1]oct-8-yl)(6-methoxypyridin-2-yl)methanone, (3-Chloro-6-methoxypyridin-2-yl)(3-{[2-(4-chlorophenyl)imidazo[1,2-a]pyrimidin-3-yl]methyl}-3,8-diazabicyclo[3.2.1]oct-8-yl)methanone, (3-{[2-(4-Chlorophenyl)imidazo[1,2-a]pyrimidin-3-yl]methyl}-3,8-diazabicyclo[3.2.1]oct-8-yl)(3-fluoro-6-methoxypyridin-2-yl)methanone, (3-Chloro-6-methoxypyridin-2-yl)(5-{[2-(4-isopropylphenyl)imidazo[1,2-a]pyrimidin-3-yl]methyl}-2,5-diazabicyclo[2.2.2]oct-2-yl)methanone, (3-Fluoro-6-methoxypyridin-2-yl)(3-{[2-(4-isopropylphenyl)imidazo[1,2-a]pyrimidin-3-yl]methyl}-3,8-diazabicyclo[3.2.1]oct-8-yl)methanone, (3-Chloro-6-methoxypyridin-2-yl)(3-{[2-(4-isopropylphenyl)imidazo[1,2-a]pyrimidin-3-yl]methyl}-3,8-diazabicyclo[3.2.1]oct-8-yl)methanone, (3-{[2-(4-cyclopropylphenyl)imidazo[1,2-a]pyrimidin-3-yl]methyl}-3,8-diazabicyclo[3.2.1]octan-8-yl)(3-fluoro-6-methoxypyridin-2-yl)methanone, (3-chloro-6-methoxypyridin-2-yl)(3-{[2-(4-cyclopropylphenyl)imidazo[1,2-a]pyrimidin-3-yl]methyl}-3,8-diazabicyclo[3.2.1]octan-8-yl)methanone, 3-{[2-(4-Chlorophenyl)imidazo[1,2-a]pyridin-3-yl]methyl}-8-oxa-3,10-diazabicyclo[4.3.1]dec-10-yl](3-fluoro-6-methoxypyridin-2-yl)methanone 3-{[2-(4-Chlorophenyl)imidazo[1,2-a]pyrimidin-3-yl]methyl}-8-oxa-3,10-diazabicyclo[4.3.1]dec-10-yl](3-fluoro-6-methoxypyridin-2 -yl)methanone, [3-{[2-(5-Chloropyridin-2-yl)imidazo[1,2-a]pyridin-3-yl]methyl}-3,9-diazabicyclo[4.2.1]non-9-yl](3-fluoro-6-methoxypyridin-2-yl)methanone, (3-Fluoro-6-methoxypyridin-2-yl)[3-{[2(4-isopropylphenyl)imidazo[1,2-a]pyrimidin-3-yl]methyl}-3,9-diazabicyclo[4.2.1]nonan-9-yl]methanone.

An another embodiment the present invention is directed to combinations of compounds of

(S)-1-((2,3-dihydrobenzo[b][1,4]dioxin-2-yl)methyl)-4-(3-([¹¹C]-methoxymethyl)pyridin-2-yl)piperazine and one or more compounds selected from the list consisting of (4-{[2-(4-Bromophenyl)imidazo[1,2-a]pyridin-3-yl]methyl}piperazin-1-yl)(cyclopentyl)methanone, (4-{[2-(4-Chlorophenyl)imidazo[1,2-a]pyridin-3-yl]methyl}piperazin-1-yl)(cyclopentyl)methanone, (4-{[2-(4-Chlorophenyl)imidazo[1,2-a]pyridin-3-yl]methyl}piperazin-1-yl)(6-methoxypyridin-2-yl)methanone, (4-{[2-(4-Bromophenyl)imidazo[1,2-a]pyridin-3-yl]methyl}piperazin-1-yl)(2-fluorophenyl)methanone, (4-{[2-(4-chlorophenyl)imidazo[1,2-a]pyridin-3-yl]methyl}piperazin-1-yl)(6-isopropoxypyridin-2-yl)methanone, (4-{[2-(4-bromophenyl)imidazo[1,2-a]pyridin-3-yl]methyl}piperazin-1-yl)(6-methoxypyridin-2-yl)methanone (4-{[2-(4-Chlorophenyl)imidazo[1,2-a]pyridin-3-yl]methyl}piperazin-1-yl)[6-(trifluoromethoxy)pyridin-2-yl]methanone, (4-{[2-(4-Chlorophenyl)imidazo[1,2-a]pyridin-3-yl]methyl}piperazin-1-yl)(3-fluoro-6-methoxypyridin-2-yl)methanone, [5-{[2-(4-Chlorophenyl)imidazo[1,2-a]pyridin-3-yl]methyl}hexahydropyrrolo[3,4-c]pyrrol-2(1H)-yl](6-methoxypyridin-2-yl)methanone, [5-{[2-(4-Isopropylphenyl)imidazo[1,2-a]pyridin-3-yl]methyl}hexahydropyrrolo[3,4-c]pyrrol-2(1H)-yl]-(6-methoxypyridin-2-yl)methanone, (3-Fluoro-6-methoxypyridin-2-yl)[5-{[2-(4-isopropylphenyl)imidazo[1,2-a]pyridin-3-yl]methyl}hexahydropyrrolo[3,4-c]pyrrol-2(1H)-yl]methanone, [5-{[2-(4-Chlorophenyl)imidazo[1,2-a]pyridin-3-yl]methyl}hexahydropyrrolo[3,4-c]pyrrol-2(1H)-yl](6-methoxy-3-methylpyridin-2-yl)methanone, (−)-[(1S,4S)-5-{[2-(4- Chlorophenyl)imidazo[1,2-a]pyridin-3-yl]methyl}-2,5-diazabicyclo[2.2.2]oct-2-yl](6-methoxypyridin-2-yl)methanone, (−)-(3-Chloro-6-methoxypyridin-2-yl)[(1S,4S)-5-{[2-(4-chlorophenyl)imidazo[1,2-a]pyridin-3-yl]methyl}-2,5-diazabicyclo[2.2.2]oct-2-yl]methanone, (−)-[(1S,4S)-5-{[2-(4-Chlorophenyl)imidazo[1,2-a]pyridin-3-yl]methyl}-2,5-diazabicyclo[2.2.2]oct-2-yl](3-fluoro-6-methoxypyridin-2-yl)methanone, (5-{[2-(5-Chloropyridin-2-yl)imidazo[1,2-a]pyridin-3-yl]methyl}-2,5- diazabicyclo[2.2.2]oct-2-yl)(3-fluoro-6-methoxypyridin-2-yl)methanone, (3-Chloro-6-methoxypyridin-2-yl)(5-{[2-(5-chloropyridin-2-yl)imidazo[1,2-a]pyridin-3-yl]methyl}-2,5-diazabicyclo[2.2.2]oct-2-yl)methanone, (−)-(5-{[2-(5-Chloropyridin-2-yl)imidazo[1,2-a]pyridin-3-yl]methyl}-2,5-diazabicyclo[2.2.2]oct-2-yl)(6-methoxypyridin-2-yl)methanone, (5-{[2-(5-Chloropyridin-2-yl)imidazo[1,2-a]pyridin-3-yl]methyl}-2,5-diazabicyclo[2.2]oct-2-yl)[6-(difluoromethoxy)pyridin-2-yl]methanone, (3-{[2-(4- Chlorophenyl)imidazo[1,2-a]pyrimidin-3-yl]methyl}-3,8-diazabicyclo[3.2.1]oct-8-yl)(6-methoxypyridin-2-yl)methanone, (3-Chloro-6-methoxypyridin-2-yl)(3-{[2-(4-chlorophenyl)imidazo[1,2-a]pyrimidin-3-yl]methyl}-3,8-diazabicyclo[3.2.1]oct-8-yl)methanone, (3-{[2-(4-Chlorophenyl)imidazo[1,2-a]pyrimidin-3-yl]methyl}-3,8-diazabicyclo[3.2.1]oct-8-yl)(3-fluoro-6-methoxypyridin-2-yl)methanone, (3-Chloro-6-methoxypyridin-2-yl)(5-{[2-(4-isopropylphenyl)imidazo[1,2-a]pyrimidin-3-yl]methyl}-2,5-diazabicyclo[2.2.2]oct-2-yl)methanone, (3-Fluoro-6-methoxypyridin-2-yl)(3-{[2-(4-isopropylphenyl)imidazo[1,2-a]pyrimidin-3-yl]methyl}-3,8-diazabicyclo[3.2.1]oct-8-yl)methanone, (3-Chloro-6-methoxypyridin-2-yl)(3-{[2-(4-isopropylphenyl)imidazo[1,2-a]pyrimidin-3-yl]methyl}-3,8-diazabicyclo[3.2.1]oct-8-yl)methanone, (3-{[2-(4-cyclopropylphenyl)imidazo[1,2-a]pyrimidin-3-yl]methyl}-3,8-diazabicyclo[3.2.1]octan-8-yl)(3-fluoro-6-methoxypyridin-2-yl)methanone, (3-chloro-6-methoxypyridin-2-yl)(3-{[2-(4-cyclopropylphenyl)imidazo[1,2-a]pyrimidin-3-yl]methyl}-3,8-diazabicyclo[3.2.1]octan-8-yl)methanone, 3-{[2-(4-Chlorophenyl)imidazo[1,2-a]pyridin-3-yl]methyl}-8-oxa-3,10-diazabicyclo[4.3.1]dec-10-yl](3-fluoro-6-methoxypyridin-2-yl)methanone, 3-{[2-(4-Chlorophenyl)imidazo[1,2-a]pyrimidin-3-yl]methyl}-8-oxa-3,10-diazabicyclo[4.3.1]dec-10-yl](3-fluoro-6-methoxypyridin-2-yl)methanone, [3-{[2-(5-Chloropyridin-2-yl]imidazo[1,2-a]pyridin-3-yl]methyl}-3,9-diazabicyclo[4.2.1non-9-yl](3-fluoro-6-methoxypyridin-2-yl)methanone, (3-Fluoro-6-methoxypyridin-2-yl)[3-{[2-(4-isopropylphenyl)imidazo[1,2-a]pyrimidin-3-yl]methyl}-3,9-diazabicyclo[4.2.1]nonan-9-yl]methanone.

An another embodiment the present invention is directed to combinations of compounds of

(S)-1-((2,3-dihydrobenzo[b][1,4]dioxin-2-yl)methyl)-4-(3-(methoxymethyl)pyridin-2-yl)piperazine and

(4-{[2-(4-Chlorophenyl)imidazo[1,2-a]pyridin-3-yl]methyl}piperazin-1-yl)(6-methoxypyridin-2-yl)methanone or (3-Chloro-6-methoxypyridin-2-yl)(3-{[2-(4-isopropylphenyl)imidazo[1,2-a]pyrimidin-3-yl]methyl}-3,8-diazabicyclo[3.2.1]oct-8-yl)methanone.

An another embodiment the present invention is directed to combinations of compounds of

(S)-1((2,3-dihydrobenzo[b][1,4]dioxin-2-yl)methyl)-4-(3-(methoxymethyl)pyridin-2-yl)piperazine and (4-{[2-(4-Chlorophenyl)imidazo[1,2-a]pyridin-3-yl]methyl}piperazin-1-yl)(6-methoxypyridin-2-yl)methanone or (3-Chloro-6-methoxypyridin-2-yl)(3- {[2-(4-isopropylphenyl)imidazol[1,2-a]pyrimidin-3-yl]methyl}-3,8-diazabicyclo[3.2.1]oct-8-yl)methanone.

The terms employed herein have the meanings indicated below. The term “at least one” employed in the meanings below refers to one or several, such as one.

The term “hydroxy”, as employed herein as such or as part of another group, refers to a OH group.

In the context of the invention, (C₁-C₆)-alkyl is a straight-chain or branched alkyl radical having I to 6 carbon atoms. Examples include: methyl, ethyl, n-propyl, isopropyl, n-butyl, isobutyl, sec-butyl, tert-butyl, n-pentyl, 2-pentyl, 3-pentyl, neopentyl, n-hexyl, 2-hexyl and 3-hexyl.

In the context of the invention, (C₁-C₄)-alkyl is a straight-chain or branched alkyl radical having 1 to 4 carbon atoms. Examples include: methyl, ethyl, n-propyl, isopropyl, n-butyl, isobutyl, sec-butyl and tert-butyl.

In the context of the invention, (C₁-C₃)-alkyl is a straight-chain or branched alkyl radical having 1 to 3 carbon atoms. Examples include: methyl, ethyl, n-propyl and isopropyl.

The term (C₁-C₆)alkoxy, as employed herein as such or as part of another group, refers to an (C₁-C₆)alkyl group, as defined herein, appended to the parent molecular moiety through an oxygen atom. Representative examples of (C₁-C₆)alkoxy include, but are not limited to, methoxy, ethoxy, n-propoxy, n-butoxy, iso-butoxy, sec-butoxy, tert-butoxy, 2,2-dimethylpropoxy, 3-methylbutoxy, and n-hexoxy.

The term “halo” or “halogen”, as employed herein as such or as part of another group, refers to fluorine, chlorine, bromine or iodine.

Mono-(C₁-C₃ -alkylamino in the context of the invention is an amino group having a straight-chain or branched alkyl substituent having 1 to 3 carbon atoms. Examples include: methylamino, ethylamino, n-propylamino and isopropylamino.

Di-(C₁-C₃)alkylamino in the context of the invention is an amino group having two identical or different straight-chain or branched alkyl substituents each having 1 to 3 carbon atoms. Examples include: N,N-dimethylamino, N,N-diethylamino, N-ethyl-N-methylamino, N-methyl-N-n-propylamino, N-isopropyl-N-methylamino, N,N-di-n-propylamino, N-isopropyl-N-n-propylamino and N,N-diisopropylamino.

(C₁-C₃)-Alkylsulfanyl [also referred to as (C₁-C₃)-alkylthio] in the context of the invention is a straight-chain or branched alkyl radical having 1 to 3 carbon atoms which is attached to the remainder of the molecule via a sulfur atom. Examples include: methylsulfanyl, ethylsulfanyl, n-propylsulfanyl and isopropylsulfanyl.

(C₃-C₆)-Cycloalkyl in the context of the invention is a monocyclic saturated cycloalkyl group having 3 to 6 ring carbon atoms. Examples include: cyclopropyl, cyclobutyl, cyclopentyl and cyclohexyl.

(C₄-C₆ -Cycloalkyl in the context of the invention is a monocyclic saturated cycloalkyl group having 4 to 6 carbon atoms. Examples include: cyclobutyl, cyclopentyl and cyclohexyl.

The term hydroxy(C₁-C₆)alkyl, as employed herein as such or as part of another group, refers to at least one hydroxy group, as defined herein, appended to the parent molecular moiety through an (C₁-C₆)alkyl group, as defined herein. Representative examples of hydroxy(C₁-C₆)alkyl include, but are not limited to, hydroxymethyl, 1-hydroxyethyl, 2-hydroxyethyl, 2,2-dihydroxyethyl, 1-hydroxypropyl, 3-hydroxypropyl, 1-hydroxy-1-methylethyl, and 1-hydroxy-1-methylpropyl.

The term (C₁-C₆)alkoxy(C₁-C₆)alkyl, as employed herein as such or as part of another group. refers to at least one (C₁-C₆)alkoxy group, as defined herein, appended to the parent molecular moiety through an (C₁-C₆)alkyl group, as defined herein. When there are several (C₁-C₆)alkoxy groups, the (C₁-C₆)alkoxy groups can be identical or different.

Representative examples of (C₁-C₆)alkoxy(C₁-C₆)alkyl include, but are not limited to, methoxymethyl, ethoxymethyl, propoxymethyl, 2-methoxyethyl, 2-ethoxyethyl, 2,2-dimethoxyethyl, 1-methyl-2-propoxyethyl, 1-methoxy-1-methylethyl, and 4-methoxybutyl.

The term hydroxy(C₁-C₆)alkoxy, as employed herein as such or as part of another group, refers to at least one hydroxy group, as defined herein, appended to the parent molecular moiety through an (C₁-C₆)alkoxy group, as defined herein. Representative examples of hydroxy(C₁-C₆)alkoxy include, but are not limited to, hydroxymethoxy, dihydroxymethoxy, 2-hydroxyethoxy, 2-hydroxypropoxy, 3-hydroxypropoxy, 2-hydroxybutoxy, and 2-hydroxy-1-methylethoxy.

The term (C₁-C₆)alkoxy(C₁-C₆)alkoxy, as employed herein as such or as part of another group, refers to at least one (C₁-C₆)alkoxy group, as defined herein, appended to the parent molecular moiety through an (C₁-C₆)alkoxy group, as defined herein. The (C₁-C₆)alkoxy groups can be identical or different. Representative examples of (C₁-C₆)alkoxy(C₁-C₆)alkoxy include, but are not limited to, methoxymethoxy, propoxymethoxy, 2-methoxyethoxy, 2-ethoxyethoxy, 2-butoxyethoxy, 2,2-dimethoxyethoxy, 1-methyl-2-propoxyethoxy, 2-methoxypropoxy and 4-methoxybutoxy.

The term halo(C₁-C₆)alkoxy, as employed herein as such or as part of another group, refers to at least one halogen, as defined herein, appended to the parent molecular moiety through an (C₁-C₆)alkoxy group, as defined herein. When there are several halogens, the halogens can be identical or different.

Representative examples of halo(C₁-C₆)alkoxy include, but are not limited to, fluoromethoxy, chloromethoxy, difluoromethoxy, trifluoromethoxy, 2-bromoethoxy, 2,2,2-trichloroethoxy, 3-bromopropoxy, 2-chloropropoxy, and 4-chlorobutoxy.

The expression “compounds of the invention” as employed herein refers to the compounds of formula I.

Pharmaceutically acceptable salts, e.g. acid addition salts, with both organic and inorganic acids, are known in the field of pharmaceuticals. Representative examples of pharmaceutically acceptable acid addition salts include, but are not limited to, chlorides, bromides, sulfates, nitrates, phosphates, sulfonates, methane sulfonates, formates, tartrates, maleates, citrates, benzoates, salicylates, ascorbates, acetates and oxalates.

Hydrates or solvates are designated according to the invention as those forms of the compounds of the formula (I) which in the solid or liquid state form a molecular compound or a complex by hydration with water or coordination with solvent molecules. Examples of hydrates are sesquihydrates, monohydrates, dihydrates or trihydrates. Equally, the hydrates or solvates of salts of the compounds according to the invention are also suitable.

Is Pharmaceutically acceptable esters. when applicable, may be prepared by known methods using pharmaceutically acceptable acids that are conventional in the field of pharmaceuticals and that retain the pharmacological properties of the free form. Nonlimiting examples of these esters include esters of aliphatic or aromatic alcohols. Representative examples of pharmaceutically acceptable esters include, but are not limited to, methyl, ethyl, n-propyl, iso-propyl, n-butyl, iso-butyl, sec-butyl, tert-butyl, and benzylesters.

The invention includes within its scope all the possible geometric isomers, e.g. Z and E isomers (cis and trans isomers), of the compounds as well as all the possible optical isomers, e.g. diastereomers and enantiomers, of the compounds. Furthermore. the invention includes in its scope both the individual isomers and any mixtures thereof, e.g. racemic mixtures. The individual isomers may be obtained using the corresponding isomeric forms of the starting material or they may be separated after the preparation of the end compound according to conventional separation methods. For the separation of optical isomers, e.g. enantiomers, from the mixture thereof, conventional resolution methods, e.g. fractional crystallization may be used.

The compounds of formula (I), their production and their action as alpha2C antagonists for the treatment of diseases or conditions of the peripheric or central nervous system are disclosed in WO-A 2010/058060 in general and especially the compounds specifically are an explicit part of the description of the present invention and are hereby incorporated by reference.

The compounds of formula (II), their production and their action as selective blockers of TASK-1 and TASK-3 channels or the treatment of of respiratory disorders, sleep-related respiratory disorders. obstructive sleep apnoea, central sleep apnoea, snoring, cardiac arrhythmias, neurodegenerative disorders, neuroinflammatory disorders and neuroimmunological disorders are disclosed in WO 2017/097792 A1, WO 2017/097671 A1, WO 2018/015196 A1, WO 2018/228907 A1 and WO 2018/228909 A1 in general and especially the compounds specifically are an explicit part of the description of the present invention and are hereby incorporated by reference.

The term effective amount as used herein refers to an amount of a compound of formula (I) that is effective for treatment and/or prophylaxis of sleep-related breathing disorders, preferably obstructive and central sleep apneas and snoring.

The present invention relates to combinations of compounds of formula (I) and compounds formula (II) according to the invention for use in a method of treatment and/or prevention of respiratory disorders, sleep-related respiratory disorders. obstructive sleep apnoea, central sleep apnoea, snoring, cardiac arrhythmias, neurodegenerative disorders. neuroinflammatory disorders and neuroimmunological disorders.

The present invention relates also to the use of combinations of compounds of formula (I) and compounds of formula (II) according to the invention for production of a medicament for treatment and/or prevention of respiratory disorders, sleep-related respiratory disorders, obstructive sleep apnoea, central sleep apnoea. snoring, cardiac arrhythmias, neurodegenerative disorders, neuroinflammatory disorders and neuroimmunological disorders, preferably obstructive and central sleep apneas and snoring.

Moreover, the present invention relates to the use of one or more selective blockers of TASK-1 and TASK-3 channels in combination with one or more α2-Adrenoceptor subtype C (alpha-2C) antagonists for preparing a pharmaceutical composition for the treatment sleep-related breathing disorders.

A further subject of the present invention is the use of a combination of compounds of formula (I) and compounds of formula (II) according to the invention with one or more other active compounds in a method for the treatment and/ or prophylaxis sleep-related breathing disorders. preferably obstructive and central sleep apneas and snoring.

A further subject of the present invention is a medicament comprising at least one a combination of compounds of formula (I) and compounds of formula (II) according to the invention in combination with one or more inert non-toxic pharmaceutically suitable excipients for use in a method for the treatment and/ or prophylaxis sleep-related breathing disorders, preferably obstructive and central sleep apneas and snoring.

The present invention further relates to a medicament comprising at least one a combination of compounds of formula (I) and compounds of formula (II) according to the invention with one or more other active compounds in combination with one or more inert non-toxic pharmaceutically suitable excipients for use in a method for the treatment and/ or prophylaxis sleep-related breathing disorders, preferably obstructive and central sleep apneas and snoring.

The present invention is also directed to a method for the treatment and/or prophylaxis of sleep-related breathing disorders, by administering systemically and/or locally a therapeutically effective amount of at least one combination of compounds of formula (I) and compounds of formula (H) or a medicament comprising at least one combination of compounds of formula (I) and compounds of formula (II) according to the invention in combination with a inert, non-toxic, pharmaceutically acceptable additive.

Combination of compounds of formula (I) and compounds of formula (II) according to the invention can be used alone or, if required, in combination with one or more other pharmacologically active substances, provided that this combination does not lead to undesirable and unacceptable side effects. Preferred examples of combination suitable for the purpose to treat sleep-related breathing disorders, preferably obstructive and central sleep apneas and snoring, include:
respiratory stimulants such as, by way of example and with preference, theophylline, doxapram, nikethamide or caffeine;
psychostimulants such as, by way of example and with preference, modafinil or armodafinil;
amphetamines and amphetamine derivatives such as, by way of example and with preference, amphetamine, metamphetamine or methylphenidate;
serotonin reuptake inhibitors such as, by way of example and with preference, fluoxetine, paroxetine, citalopram, escitalopram, sertraline, fluvoxamine or trazodone;
serotonin precursors such as, by way of example and with preference, L-tryptophan;
selective serotonin noradrenaline reuptake inhibitors such as, by way of example and with preference, venlafaxine or duloxetine;
noradrenergic and specific serotonergic antidepressants such as, by way of example and with preference, mirtazapine,
selective noradrenaline reuptake inhibitors such as, by way of example and with preference, reboxetine or atomoxetine;
tricyclic antidepressants such as, by way of example and with preference, amitriptyline, protriptyline doxepine trimipramine, imipramine, clomipramine or desipramine;
muscarinic receptor antagonists, by way of example and with preference oxybutynin;
GABA agonists such as, by way of example and with preference, baclofen;
glucocorticoids such as, by way of example and with preference, fluticasone, budesonide, beclometasone, mometasone, tixocortol or triamcinolone;
cannabinoid receptor agonists;
carboanhydrase inhibitors such as, by way of example and with preference, acetazolamide, methazolamide or diclofenamide;
opioid and benzodiazepine receptor antagonists such as, by way of example and with preference, flumazenil, naloxone or naltrexone:
cholinesterase inhibitors such as, by way of example and with preference, neostigmine, pyridostigmine, physostigmine donepezil, galantamine or rivastiginine,
appetite suppressants such as, by way of example and with preference, sibutramin, opiramate, phentennine, lipase inhibitors or cannabinoid receptor antagonists;
mineralocorticoid receptor antagonists.

Medicament comprising combinations as defined in any of claims 1 to 5 in combination with one or more further active ingredients selected from the group consisting of muscarinic receptor antagonists, mineralocorticoid receptor antagonists, diuretics, corticosteroids.

A preferred subject of the present invention is a combination comprising combinations of compounds of formula (I) and compounds of formula (II) according to the invention and one or more other active compounds selected from the groups consisting of muscarinic receptor antagonists, mineralocorticoid receptor antagonists, diuretics, corticosteroids for use in a method for the treatment and/ or prophylaxis sleep-related breathing disorders, preferably obstructive and central sleep apneas and snoring.

Another preferred subject of the present invention is a medicament comprising combinations of compounds of formula (I) and compounds of formula (II) according to the invention in combination with one or more other active compounds selected from the groups consisting of muscarinic receptor antagonists.

In a preferred embodiment of the invention, the combinations of the invention are administered in combination with a muscarinic receptor antagonist, by way of example and with preference oxybutynin.

In a preferred embodiment of the invention, the combinations of the invention are administered in combination with a mineralocorticoid receptor antagonist, by way of example and with preference spironolactone, eplerenone or finerenone.

In a preferred embodiment of the invention, the combinations of the invention are administered in combination with a diuretic, by way of example and with preference furosemide, bumetanide, torsemide, bendroflumethiazide, chlorothiazide, hydrochlorothiazide, hydroflumethiazide, methyclothiazide, polythiazide, trichlonnethiazide, chlorthalidone, indapamide, metolazone, quinethazone, acetazolamide, dichlorphenamide, methazolamide, glycerol, isosorbide, mannitol, amiloride or triamterene.

In a preferred embodiment of the invention, the compounds of the invention are administered in combination with a corticosteroid, by way of example and with preference prednisone, prednisolone, methylprednisolone, triamcinolone, dexainethasone, betamethasone, beclomethasone, flunisolide, budesonide or fluticasone.

If required, aryl piperazines of formula (I) according to the invention can also be employed in conjunction with the use of one or more medical technical devices or auxiliaries, provided this does not lead to unwanted and unacceptable side-effects. Medical devices and auxiliaries suitable for such a combined application are, by way of example and with preference:
devices for positive airway pressure ventilation such as, by way of example and with preference, CPAP (continuous positive airway pressure) devices, BiPAP (bilevel positive airway pressure) devices and IPPV (intermittent positive pressure ventilation) devices;
neurostimulators of the Nervus hypoglossus:
intraoral auxiliaries such as, by way of example and with preference, protrusion braces;
nasal disposable valves;
nasal stents.

Aryl piperazines of formula (I) and compounds of formula (II) according to the invention can act systemically and/or locally. For this purpose. they can be administered in a suitable manner, for example by the oral, parenteral, pulmonal, intrapulmonal (inhalative), nasal, intranasal, pharyngeal, lingual, sublingual, buccal, rectal, dermal, transdermal, conjunctival or otic route, or as an implant or stent.

A further subject of the present invention is a pharmaceutical composition comprising a combination of a compound of the formula (I) and a compound of formula (II) according to the invention for the systemic and/or local administration by the oral, parenteral, pulmonal, intrapulmonal (inhalative), nasal, intranasal, pharyngeal, lingual, sublingual, buccal, rectal, dermal, transdermal, conjunctival or otic route, or as an implant or stent. The preferred administrations are the oral, nasal and pharyngeal routes.

For these administration routes, the compounds according to the invention can be administered in suitable administration forms.

For oral administration, administration forms which function according to the state of the art, releasing the compounds according to the invention rapidly and/or in a modified manner, which contain the compounds according to the invention in crystalline and/or amorphized and/or dissolved form, such as for example tablets (uncoated or coated tablets, for example with gastric juice-resistant or delayed dissolution or insoluble coatings, which control the release of the compound according to the invention), tablets rapidly disintegrating in the oral cavity or films/wafers, films/lyophilisates, capsules (for example hard or soft gelatine capsules), dragees, granules, pellets, powders, emulsions, suspensions, aerosols or solutions are suitable.

Parenteral administration can be effected omitting an absorption step (e.g. intravenous, intra-arterial, intracardial, intraspinal or intralumbar administration) or involving absorption (e.g. intra-muscular, subcutaneous, intracutaneous, percutaneous or intraperitoneal administration). Suitable administration forms for parenteral administration include injection and infusion preparations in the form of solutions, suspensions, emulsions, lyophilisates or sterile powders.

For the other administration routes, for example inhalation formulations (including powder inhalers and nebulisers), nasal drops, solutions or sprays, pharyngeal sprays, tablets for lingual, sublingual or buccal administration. tablets, films/wafers or capsules, suppositories, oral or ophthalmic preparations, vaginal capsules, aqueous suspensions (lotions, shakable mixtures), lipophilic suspensions, ointments, creams, transdermal therapeutic systems (e.g. plasters), milk, pastes. foams, dusting powders, implants or stents are suitable.

Oral or nasal and pharyngeal administration are preferred.

The compounds according to the invention can be converted into the stated administration forms. This can be effected in a manner known per se by mixing with inert. non-toxic, pharmaceutically suitable additives. These additives include carriers (for example microcrystalline cellulose, lactose, mannitol), solvents (e.g. liquid polyethylene glycols), emulsifiers and dispersants or wetting agents (for example sodium dodecylsulphate, polyoxysorbitan oleate), binders (for example polyvinylpyrrolidone), synthetic and natural polymers (for example albumin), stabilizers (e.g. antioxidants such as for example ascorbic acid), colourants (e.g. inorganic pigments such as for example iron oxides) and flavour or odour correctors.

In general, to achieve effective results in oral administration it has been found advantageous to administer quantities of about 0.01 to 100 mg/kg, preferably about 0.01 to 10 mg/kg body weight. In nasal or pharyngeal administration, the dosage is about 0.01 μg/kg to 1000 μg/kg, preferably about 0.1 to 10 μg/kg body weight. Nonetheless it can sometimes be necessary to deviate from the said quantities, namely depending on body weight, administration route, individual response to the active substance, nature of the preparation and time or interval at which administration takes place. Thus in some cases it can be sufficient to manage with less than the aforesaid minimum quantity, while in other cases the stated upper limit must be exceeded. In the event of administration of larger quantities, it may be advisable to divide these into several individual administrations through the day.

A further subject of the present invention is the combination of the systemic administration of a compound of formula (I) with the local administration of a compound of formula (II).

For this purpose, compound of formula (I) can be administered in a suitable manner, for example by the oral, parenteral, pulmonal, intrapulmonal (inhalative), nasal, intranasal, pharyngeal, lingual, sublingual, buccal, rectal, dermal, transdermal, conjunctival or otic route, or as an implant or stent and compounds of formula (II) can be administered for example by the nasal, intranasal, pharyngeal, lingual, sublingual, and buccal route.

The preferred administration is the oral route for a compound of of formula (I) and the nasal and pharyngeal route for a compound of formula (II).

For oral administration, administration forms which function according to the state of the art, releasing the compounds according to the invention rapidly and/or in a modified manner, which contain the compounds according to the invention in crystalline and/or amorphized and/or dissolved form, such as for example tablets (uncoated or coated tablets. for example with gastric juice-resistant or delayed dissolution or insoluble coatings, which control the release of the compound according to the invention), tablets rapidly disintegrating in the oral cavity or films/wafers, films/lyophilisates, capsules (for example hard or soft gelatine capsules), dragees, granules, pellets, powders, emulsions, suspensions, aerosols or solutions are suitable.

For the nasal and pharyngeal administration routes, for example nasal drops, solutions or sprays, pharyngeal sprays, tablets for lingual, sublingual or buccal administration, tablets, films/wafers or capsules, suppositories or oral preparations are suitable.

The following practical examples illustrate the invention. The invention is not limited to the examples.

### EXAMPLES

### A. Experimental Methods

Advantageous pharmacological properties of the combination of an α2-Adrenoceptor subtype C (alpha-2C) antagonists with a TASK1/3 channel blocker can be determined by the following methods.

The therapeutic potential of the the combination of an α2-Adrenoceptor subtype C (alpha-2C) antagonists with a TASK1/3 channel blocker according to the present invention in sleep apnea has been assessed preclinically in a pig model of obstructive sleep apnea (OSA).

Using negative pressure. it is possible to induce collapse and thus obstruction of the upper respiratory tract in anaesthetized, spontaneously breathing pigs (Wirth K. J. et al., Sleep 36(5) (2013) pp. 699-708).

German Landrace pigs are used for the model. The pigs are anaesthetized and tracheotomized. Two tracheal cannulas are inserted into the trachea, one into the rostral part and the other into the caudal part of the trachea. Using a connection piece. the rostral cannula is connected to a tube to the negative pressure device and to the distal tracheal cannula. The distal tracheal cannula is additionally connected to a tube with an open end to atmosphere via a connection piece that served for free tracheal breathing, circumventing the upper airway. By appropriate opening and clamping of those tubes breathing can be switched from nasal breathing to breathing through the caudal tracheal cannula, circumventing the upper airway. and the (isolated) upper airway can be connected to the negative pressure device, causing airflow in the inspiratory direction.

At certain points in time. the collapsibility of the upper respiratory tract is tested by having the pig breathe via the caudal cannula and applying negative pressures of −50, −100 and −150 cm water head (cm H₂O) to the upper respiratory tract. This causes the upper respiratory tract to collapse, which manifests itself in an interruption of the airflow and a pressure drop in the tube system. This test is conducted prior to the administration of the test substance and at certain intervals after the administration of the test substance. An appropriately effective test substance can prevent this collapse of the respiratory tract in the inspiratory phase.

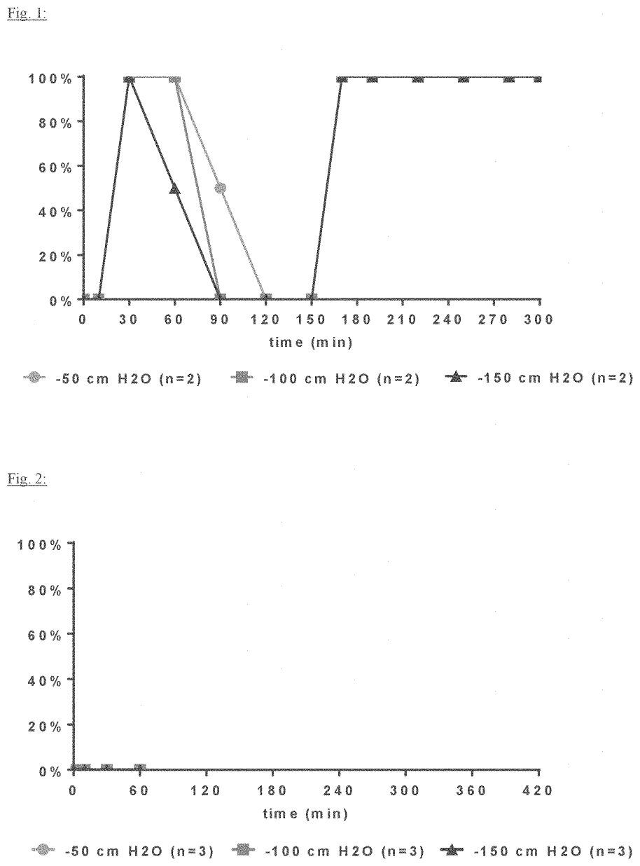

In this OSA pig model, systemic application of the a2-Adrenoceptor subtype C (alpha-2C) antagonists of formula (I) ((S)-1-((2,3-dihydrobenzo[b][1,4]dioxin-2-yl)methyl)-4-(3-(methoxymethyl)pyridin-2-yl)piperazine with i.v. bolus injection of 0.3μg/kg followed by an i.v. infusion of 0.13 μg/kg/h for five hours inhibited upper airway collapsibility at all negative pressures of −50, −100 and −150 cm head (cm H₂O) only at time point 30 min after bolus injection and start of infusion. At time point 120 min after bolus injection and start of infusion, upper airway collapsibility was induced at all negative pressures of −50, −100 and −150 cm head (cm H2O). The combination of this non effective dose of the α2-Adrenoceptor subtype C (alpha-2C) antagonists of formula (I) ((S)-1-((2,3-dihydrobenzo[b][1,4]dioxin-2-yl)methyl)-4-(3-(methoxymethyl)pyridin-2-yl)piperazine with the non effective dose of the TASK1/TASK3 channel blocker of 0.3 μg (4-{[2-(4-Chlorophenypimidazo[1,2-a]pyridin-3-yl]methyl}piperazin-1-yl)(6-methoxypyridin-2-yl)methanone inhibits upper airway collapsibility at all negative pressures of −50, 100 and −150 cm head (cm H₂O) for more than two hours (see Table 1, 2 and 3 and FIG. 1).

FIG. 1: Effect of i.v. bolus injection of 0.13 μg/kg followed by an i.v. infusion of 0.13 μg/kg/h for five hours of the α2-Adrenoceptor subtype C (alpha-2C) antagonists of formula (I) ((S)-1-((2,3-dihydrobenzo[b][1,4]dioxin-2-yl)methyl)-4-(3-(methoxymethyl)pyridin-2-yl)piperazine given at time point 0 min in combination with intranasal administration of 0.3 μg of the TASK1/TASK3 channel blocker (4-{[2-(4-Chlorophenyl)imidazo[1,2-a]pyridin-3 -yl]methyl}piperazin-1-yl)(6-methoxypyridin-2-yl)methanone given at time point 160 min after beginning of the experiment on upper airway collapsibility at different levels of negative pressure. Percentages of pigs with no collapse are given. Mean values.

TABLE 1
Combination of non effective dose of ((S)-1-((2,3-dihydrobenzo[b][1,4]dioxin-2-
yl)methyl)-4-(3-(methoxymethyl)pyridin-2-yl)piperazine with the non effective dose of
0.3 μg (4-{[2-(4-Chlorophenyl)imidazo[1,2-a]pyridin-3-yl]methyl}piperazin-1-yl)(6-
methoxypyridin-2-yl)methanone inhibits upper airway collapsibility at negative
pressures of −50 cm head (cm H₂O)

| | | Percent pigs without |
|---|---|---|
| | Time, min | collaps −50 cm H₂O, % |
| | 0 | 0 |
| | 10 | 0 |
| | 30 | 100 |
| | 60 | 100 |
| | 90 | 50 |
| | 120 | 0 |
| | 150 | 0 |
| Nasal application TASK1/3 | 160 | |
| channel blocker | | |
| | 170 | 100 |
| | 190 | 100 |
| | 220 | 100 |
| | 250 | 100 |
| | 280 | 100 |
| | 300 | 100 |

TABLE 2
Combination of non effective dose of ((S)-1-((2,3-dihydrobenzo[b][1,4]dioxin-2-
yl)methyl)-4-(3-(methoxymethyl)pyridin-2-yl)piperazine with the non effective dose of
0.3 μg (4-{[2-(4-Chlorophenyl)imidazo[1,2-a]pyridin-3-yl]methyl}piperazin-1-yl)(6-
methoxypyridin-2-yl)methanone inhibits upper airway collapsibility at negative
pressures of −100 cm head (cm H₂O)

| | Time, min | Percent pigs without collaps −100 cm H₂O, % |
|---|---|---|
| | 0 | 0 |
| | 10 | 0 |
| | 30 | 100 |
| | 60 | 100 |
| | 90 | 0 |
| | 120 | 0 |
| | 150 | 0 |
| Nasal application TASK1/3 | 160 | |
| channel blocker | | |
| | 170 | 100 |
| | 190 | 100 |
| | 220 | 100 |
| | 250 | 100 |
| | 280 | 100 |
| | 300 | 100 |

TABLE 3
Combination of non effective dose of ((S)-1-((2,3-dihydrobenzo[b][1,4]dioxin-2-
yl)methyl)-4-(3-(methoxymethyl)pyridin-2-yl)piperazine with the non effective dose of
0.3 μg (4-{[2-(4-Chlorophenyl)imidazo[1,2-a]pyridin-3-yl]methyl}piperazin-1-yl)(6-
methoxypyridin-2-yl)methanone inhibits upper airway collapsibility at negative
pressures of −150 cm head (cm H₂O)

| | Time, min | Percent pigs without collaps −150 cm H₂O, % |
|---|---|---|
| | 0 | 0 |
| | 10 | 0 |
| | 30 | 100 |
| | 60 | 50 |
| | 90 | 0 |
| | 120 | 0 |
| | 150 | 0 |
| Nasal application TASK1/3 | 160 | |
| channel blocker | | |
| | 170 | 100 |
| | 190 | 100 |
| | 220 | 100 |
| | 250 | 100 |
| | 280 | 100 |
| | 300 | 100 |

Table 4, 5 and 6 and FIG. 2: Effect of intranasal administration of 0.3 μg of the TASK1/TASK3 channel blocker (4-{[2-(4-Chlorophenyl)imidazo[1,2-a]pyridin-3-yl]methyl}piperazin-1 -yl)(6-methoxypyridin-2-yl)methanone given at time point 0 min on upper airway collapsibility at different levels of negative pressure. Percentages of pigs with no collapse are given. Mean values.

TABLE 4
Intranasal administration of 0.3 μg of the TASK1/TASK3 channel blocker (4-{[2-(4-
Chlorophenyl)imidazo[1,2-a]pyridin-3-yl]methyl}piperazin-1-yl)(6-methoxypyridin-2-
yl)methanone at negative pressures of −50 cm head (cm H₂O)

| Time, min | Percent pigs without collaps −50 cm H₂O, % |
|---|---|
| 0 | 0 |
| 10 | 0 |
| 30 | 0 |
| 60 | 0 |

TABLE 5
Intranasal administration of 0.3 μg of the TASK1/TASK3 channel blocker (4-{[2-(4-
Chlorophenyl)imidazo[1,2-a]pyridin-3-yl]methyl}piperazin-1-yl)(6-methoxypyridin-2-
yl)methanone at negative pressures of −100 cm head (cm H₂O)

| Time, min | Percent pigs without collaps −100 cm H₂O, % |
|---|---|
| 0 | 0 |
| 10 | 0 |
| 30 | 0 |
| 60 | 0 |

TABLE 6
Intranasal administration of 0.3 μg of the TASK1/TASK3 channel blocker (4-{[2-(4-
Chlorophenyl)imidazo[1,2-a]pyridin-3-yl]methyl}piperazin-1-yl)(6-methoxypyridin-2-
yl)methanone at negative pressures of −150 cm head (cm H₂O)

| Time, min | Percent pigs without collaps −150 cm H₂O, % |
|---|---|
| 0 | 0 |
| 10 | 0 |
| 30 | 0 |
| 60 | 0 |

From the above mentioned data it can be deducted that the combination of an α2-Adrenoceptor subtype C (alpha-2C) antagonists of formula (I) with a TASK 1/3 channel blocker inhibits upper airway collapsibility with improved efficacy compared to each treatment alone and is thus suitable to treat sleep-related breathing disorders, preferably obstructive and central sleep apneas and snoring.

1. A combination comprising a compound of formula (I) 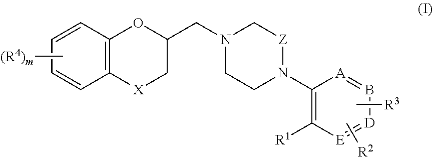
wherein
X is O, S or CH₂;
Z is —[CH₂]ₙ—;
A, B, D and E are independently C or N, provided that at least three of A, B, D and E are C;
R¹ is H, halogen, hydroxy, (C₁-C₆)alkyl, (C₁-C₆)alkoxy, hydroxy(C₁-C₆)alkyl, (C₁-C₆)alkoxy(C₁-C₆)alkyl, halo(C₁-C₆)alkoxy, halo(C₁-C₆)alkoxy(C₁-C₆)alkyl, hydroxy(C₁-C₆)alkoxy(C₁-C₆)alkyl, (C₁-C₆)alkoxy(C₁-C₆)alkoxy(C₁-C₆)alkyl, (C₁-C₆)alkoxy-(C═O)—, CN, (R₅)₂N—, (R₅)₂N—(C₁-C₆)alkyl, (R₅)₂N-(C═O)—, SH—(C₁-C₆)alkyl, hydroxy(C₁-C₆)alkyl-S-(C₁-C₆)alkyl, (C₁-C₆)alkoxy(C₁-C₆)alkyl-S—(C₁-C₆)alkyl, hydroxy(C₁-C₆)alkyl-S(Op)-(C₁-C₆)alkyl, (C₁-C₆)alkoxy(C₁-C₆)alkyl-S(Op)-(C₁-C₆)alkyl or furyl;
R² is H, halogen, (C₁-C₆)alkyl, (C₁-C₆)alkoxy or hydroxy(C₁-C₆)alkyl;
R³ is H, halogen, (C₁-C₆)alkyl or phenyl;
R⁴ is halogen, hydroxy, (C₁-C₆)alkyl, (C₁-C₆)alkoxy, CN or (R⁵)₂N—;
R⁵ is, independently at each occurence, H, (C₁-C₆)alkyl or (C₁-C₆)alkoxy(C₁-C₆)alkyl;
m is 0, 1 or 2;
n is 1 or 2; and
p is 1 or 2,
or a salt, solvate, or solvate of a salt thereof,
and a compound of formula (II) 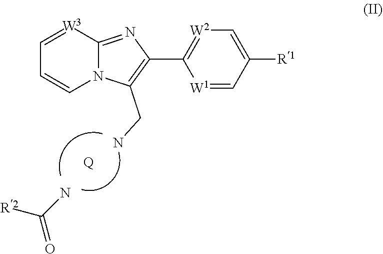
wherein
the ring Q represents a piperazine or a diazaheterobicyclic system of the formula 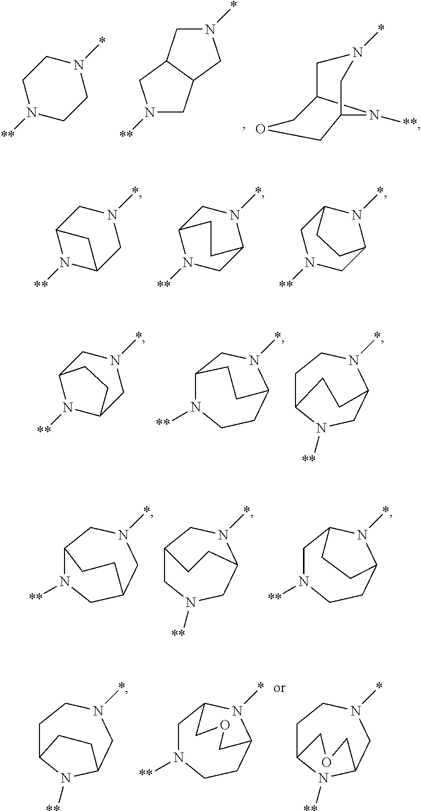
wherein * denotes a bond to the adjacent CHR′² group and ** a bond to the carbonyl group;
W¹, W² and W³ are each independently CH or N;
R′¹ is halogen, cyano, (C₁-C₄)-alkyl, cyclopropyl or cyclobutyl wherein (C₁-C₄)-alkyl may be up to trisubstituted by fluorine and cyclopropyl and cyclobutyl may be up to disubstituted by fluorine;
and
R′² is (C₄-C₆)-cycloalkyl in which a ring CH₂ group may be replaced by —O—;
or
R′² a phenyl group of the formula (a), a pyridyl group of the formula (b) or (c) or an azole group of the formula (d), (e), (f) or (g); 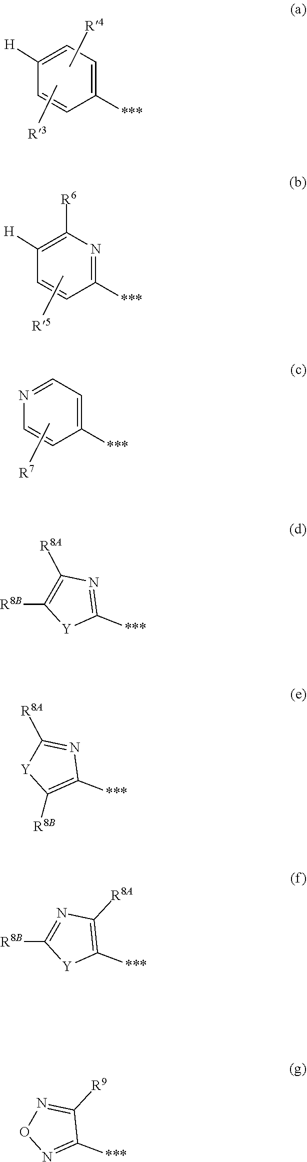
wherein *** marks a bond to the adjacent carbonyl group;
R′³ is hydrogen, fluorine, chlorine, bromine or methyl;
R′⁴ is hydrogen, fluorine, chlorine, bromine, cyano, (C₁-C₃)-alkyl or (C₁-C₃)-alkoxy;
wherein (C₁-C₃)-alkyl and (C₁-C₃)-alkoxy may each be up to trisubstituted by fluorine,
R′⁵ is hydrogen, fluorine, chlorine, bromine or methyl;
R′⁶ is hydrogen, (C₁-C₃)-alkoxy, cyclobutyloxy, oxetan-3-yloxy, tetrahydrofuran-3-yloxy, tetrahydro-2H-pyran-4-yloxy, mono-(C₁-C₃)-alkylamino, di-(C₁-C₃)-alkylamino or (C₁-C₃)-alkylsulfanyl[H];
wherein (C₁-C₃)-alkoxy may be up to trisubstituted by fluorine,
R⁷ is hydrogen, fluorine, chlorine, bromine, (C₁-C₃)-alkyl or (C₁-C₃)-alkoxy;
R^{8A} and R^{8B} are identical or different and independently of one another are hydrogen, fluorine, chlorine, bromine, (C₁-C₃)-alkyl, cyclopropyl or (C₁-C₃)-alkoxy
wherein (C₁-C₃)-alkyl and (C₁-C₃)-alkoxy may each be up to trisubstituted by fluorine;
R⁹ is hydrogen, (C₁-C₃)-alkyl or amino;
and
wherein in subformula (d)
Y is O, S or N(CH₃);
wherein in subformula (e) and (f),
Y is O or S,
or
R′² is OR¹⁰ or —NR¹¹R¹², wherein
R¹⁰ is (C₁-C₆)-alkyl, (C₄-C₆)-cycloalkyl or [(C₃-C₆)-cycloalkyl]methyl,
R¹¹ is hydrogen or (C₁-C₃)-alkyl
and
R¹² is (C₁-C₆)-alkyl, (C₃-C₆)-cycloalkyl, phenyl or benzyl, 1-phenylethyl or 2-phenylethyl,
wherein (C₁-C₆)-alkyl may be up to trisubstituted by fluorine,
and
wherein phenyl and a phenyl group in benzyl, 1-phenylethyl and 2-phenylethyl may be up to trisubstituted by identical or different radicals selected from the group consisting of fluorine, chlorine, methyl, ethyl, trifluoromethyl, methoxy, ethoxy, trifluoromethoxy and (trifluoromethyl)sulfanyl,
or
R¹¹ and R¹² are attached to one another and, together with the nitrogen atom to which they are bonded, form a pyrrolidine, piperidine, morpholine or thiomorpholine ring, or
R¹¹ and R¹² are attached to one another and, together with the nitrogen atom to which they are bonded, form a tetrahydroquinoline ring of formula (c) or a tetrahydroisoquinoline ring of formula (d), 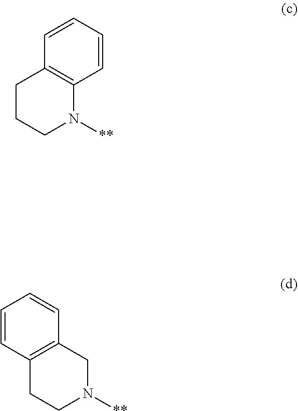
wherein ** marks a bond to the carbonyl group,
or a salt, solvate, or solvate of a salt thereof.

2. The combination of claim 1, wherein
X is O;
Z is —[CH₂]ₙ—;
A is N;
B, D and E are C;
R¹ is halogen, (C₁-C₆)alkyl, (C₁-C₆)alkoxy, hydroxy(C₁-C₆)alkyl, (C₁-C₆)alkoxy(C₁-C₆)alkyl, halo(C₁-C₆)alkoxy, halo(C₁-C₆)alkoxy(C₁-C₆)alkyl, (C₁-C₆)alkoxy-(C═O)—, CN, (R₅)₂N-(C₁-C₆)alkyl, (R₅)₂N—(C═O)— or furyl;
R² is H, halogen, (C₁-C₆)alkyl or hydroxy(C₁-C₆)alkyl;
R³ is H, (C₁-C₆)alkyl or phenyl;
R⁵ is, independently at each occurence, H or (C₁-C₆)alkyl;
m is 0; and
n is 1
and wherein
the ring Q represents a piperazine or a diazaheterobicyclic system of the formula 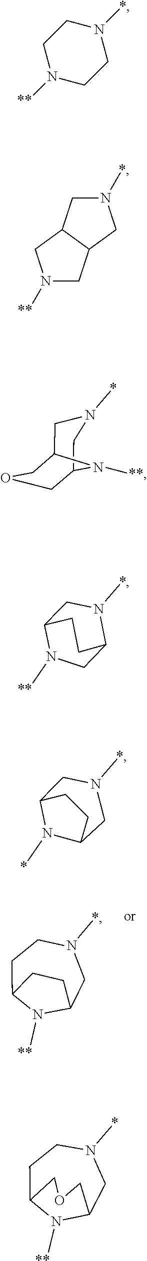
wherein * denotes a bond to the adjacent CHR² group and ** a bond to the carbonyl group,
W² is CH,
W¹ and W³ are independently CH or N,
R′¹ is fluorine, chlorine, bromine, methyl, tert.-butyl, isopropyl, cyclopropyl or cyclobutyl,
and
R′² is cyclobutyl, cyclopentyl or cyclohexyl,
or
R′² is a phenyl group of the formula (a), a pyridyl group of the formula (b) or an azole group of the formula (d) or formula (g) 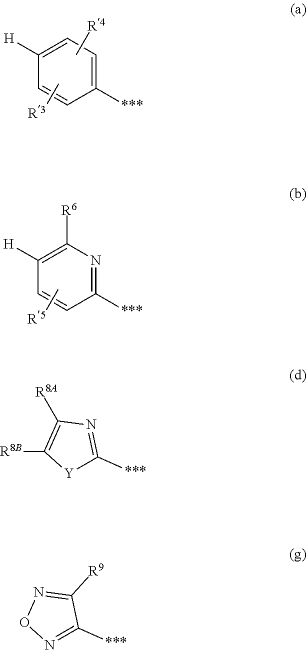
wherein *** marks a bond to the adjacent carbonyl group and
R′³ is hydrogen, fluorine or chlorine,
R′⁴ is fluorine, chlorine, methyl, isopropyl, methoxy or ethoxy,
R′⁵ is hydrogen, fluorine, chlorine, bromine or methyl,
R⁶ is methoxy, difluoromethoxy, trifluoromethoxy, isopropoxy, cyclobutyloxy or methylsulfanyl,
R^{8A} and R^{8B} are independently hydrogen, methyl, trifluoromethyl, ethyl, isopropyl or cyclopropyl,
and
R⁹ is methyl or amino
Y is O or S or N(CH₃)
or a salt, solvate, or solvate of a salt thereof.

3. The combination of claim 1, wherein the compound of formula (I) is selected from the group consisting of:
(S)-1-((2,3-Dihydrobenzo[b][1,4]dioxin-2-yl)methyl)-4-(2-(methoxymethyl)phenyl)piperazine,
(R)-1-((2,3-Dihydrobenzo[b][1,4]dioxin-2-yl)methyl)-4-(2-(methoxymethyl)phenyl)piperazine,
(2-(4-((2,3-Dihydrobenzo[b][1,4]dioxin-2-yl)methyl)piperazin-1-yl)-6-fluorophenyl)methanol,
(S)-1-((2,3-Dihydrobenzo[b][1,4]dioxin-2-yl)methyl)-4-(2-(furan-2-yl)phenyl)piperazine,
(S)-1-((2,3-Dihydrobenzo[b][1,4]dioxin-2-yl)methyl)-4-o-tolylpiperazine, methyl 2-(4-((2,3-dihydrobenzo[b][1,4]dioxin-2-yl)methyl)-1,4-diazepan-1-yl)benzoate, and
(S)-1-((2,3-dihydrobenzo[b][1,4]dioxin-2-yl)methyl)-4-(3-(methoxymethyl)pyridin-2-yl)piperazine
and the compound of formula (II) is selected from the group consisting of:
(4-{[2-(4-Bromophenyl)imidazo[1,2-a]pyridin-3-yl]methyl}piperazin-1-yl)(cyclopentyl)methanone,
(4-{[2-(4-Chlorophenyl)imidazo[1,2-a]pyridin-3-yl]methyl}piperazin-1-yl)(cyclopentyl)methanone,
(4-{[2-(4-Chlorophenyl)imidazo[1,2-a]pyridin-3-yl]methyl}piperazin-1-yl)(6-methoxypyridin-2-yl)methanone,
(4-{[2-(4-Bromophenyl)imidazo[1,2-a]pyridin-3- yl]methyl}piperazin-1yl)(2-fluorophenyl)methanone,
(4-{[2-(4-chlorophenyl)imidazo[1,2-a]pyridin-3-yl]methyl}piperazin-1-yl)(6-isopropoxypyridin-2-yl)methanone,
(4-{[2-(4-bromophenyl)imidazo[1,2-a]pyridin-3-yl]methyl}piperazin-1-yl)(6-methoxypyridin-2-yl)methanone,
(4-{[2-(4-Chlorophenyl)imidazo[1,2-a]pyridin-3-yl]methyl}piperazin-1-yl)[6-(trifluoromethoxy)pyridin-2-yl]methanone,
(4-{[2-(4-Chlorophenyl)imidazo[1,2-a]pyridin-3-yl]methyl}piperazin-1-yl)(3-fluoro-6-methoxypyridin-2-yl)methanone,
[5-{[2-(4-Chlorophenyl)imidazo[1,2-a]pyridin-3-yl]methyl}hexahydropyrrolo[3,4-c]pyrrol-2(1H)-yl](6-methoxypyridin-2-yl)methanone,
[5-{[2-(4-Isopropylphenyl)imidazo[1,2-a]pyridin-3-yl]methyl}hexahydropyrrolo[3,4-c]pyrrol-2(1H)-yl](6-methoxypyridin-2-yl)methanone,
(3 -Fluoro-6-methoxypyridin-2-yl)[5-{[2-(4-isopropylphenyl)imidazo[1,2-a]pyridin-3-yl]methyl}hexahydropyrrolo[3,4-c]pyrrol-2(1H)-yl]methanone,
[5-{[2-(4-Chlorophenyl)imidazo[1,2-a]pyridin-3-yl]methyl}hexahydropyrrolo[3,4-c]pyrrol-2(1H)-yl](6-methoxy-3-methylpyridin-2-yl)methanone,
(−)-[(1S,4S)-5-{[2-(4-Chlorophenyl)imidazo[1,2-a]pyridin-3-yl]methyl}-2,5-diazabicyclo[2.2.2]oct-2-yl](6-methoxypyridin-2-yl)methanone,
(−)-(3-Chloro-6-methoxypyridin-2-yl)[(1S,4S)-5-{[2-(4-chlorophenyl)imidazo[1,2-a]pyridin-3-yl]methyl}-2,5-diazabicyclo[2.2.2]oct-2-yl]methanone,
(−)-[(1S,4S)-5-{[2-(4-Chlorophenyl)imidazo[1,2-a]pyridin-3-yl]methyl}-2,5-diazabicyclo[2.2.2]oct-2-yl](3-fluoro-6-methoxypyridin-2-yl)methanone,
(5-{[2-(5-Chloropyridin-2-yl)imidazo[1,2-a]pyridin-3-yl]methyl}-2,5-diazabicyclo[2.2.2]oct-2-yl)(3-fluoro-6-methoxypyridin-2-yl)methanone,
(3-Chloro-6-methoxypyridin-2-yl)(5-{[2-(5-chloropyridin-2-yl)imidazo[1,2-a]pyridin-3-yl]methyl}-2,5-diazabicyclo[2.2.2]oct-2-yl)methanone,
(−)-(5-{[2-(5-Chloropyridin-2-yl)imidazo[1,2-a]pyridin-3-yl]methyl}-2,5-diazabicyclo[2.2.2]oct-2-yl)(6-methoxypyridin-2-yl)methanone,
(5-{[2-(5-Chloropyridin-2-yl)imidazo[1,2-a]pyridin-3-yl]methyl}-2,5-diazabicyclo[2.2.2]oct-2-yl)[6-(difluoromethoxy)pyridin-2-yl]methanone,
(3-{[2-(4-Chlorophenyl)imidazo[1,2-a]pyrimidin-3-yl]methyl}-3,8-diazabicyclo[3.2.1]oct-8-yl)(6-methoxypyridin-2-yl)methanone,
(3-Chloro-6-methoxypyridin-2-yl)(3-{[2-(4-chlorophenyl)imidazo[1,2-a]pyrimidin-3-yl]methyl}-3,8-diazabicyclo[3.2.1]oct-8-yl)methanone,
(3-{[2-(4-Chlorophenyl)imidazo[1,2-a]pyrimidin-3-yl]methyl}-3,8-diazabicyclo[3.2.1]oct-8-yl)(3-fluoro-6-methoxypyridin-2-yl)methanone,
(3-Chloro-6-methoxypyridin-2-yl)(5-{[2-(4-isopropylphenyl)imidazo[1,2-a]pyrimidin-3-yl]methyl}-2,5-diazabicyclo[2.2.2]oct-2-yl)methanone,
(3-Fluoro-6-methoxypyridin-2-yl)(3-{[2-(4-isopropylphenyl)imidazo[1,2-a]pyrimidin-3-yl]methyl}-3,8-diazabicyclo[3.2.1]oct-8-yl)methanone,
(3-Chloro-6-methoxypyridin-2-yl)(3-{[2-(4-isopropylphenyl)imidazo[1,2-a]pyrimidin-3-yl]methyl}-3,8-diazabicyclo[3.2.1]oct-8-yl)methanone,
(3-{[2-(4-cyclopropylphenyl)imidazo[1,2-a]pyrimidin-3-yl]methyl}-3,8-diazabicyclo[3.2.1]octan-8-yl)(3-fluoro-6-methoxypyridin-2-yl)methanone,
(3-chloro-6-methoxypyridin-2-yl)(3-{[2-(4-cyclopropylphenyl)imidazo[1,2-a]pyrimidin-3-yl]methyl}-3,8-diazabicyclo[3.2.1]octan-8-yl)methanone,
3-{[2-(4-Chlorophenyl)imidazo[1,2-a]pyridin-3-yl]methyl}-8-oxa-3,10-diazabicyclo[4.3.1]dec-10-yl](3-fluoro-6-methoxypyridin-2-yl)methanone,
3-{[2-(4-Chlorophenyl)imidazo[1,2-a]pyrimidin-3-yl]methyl}-8-oxa-3,10-diazabicyclo[4.3.1]dec-10-yl](3-fluoro-6-methoxypyridin-2-yl)methanone,
[3-{[2-(5-Chloropyridin-2-yl)imidazo[1,2-a]pyridin-3-yl]methyl}-3,9-diazabicyclo[4.2.1]non-9-yl](3-fluoro-6-methoxypyridin-2-yl)methanone, and
(3-Fluoro-6-methoxypyridin-2-yl)[3-{[2-(4-isopropylphenyl)imidazo[1,2-a]pyrimidin-3-yl]methyl}-3,9-diazabicyclo[4.2.1]nonan-9-yl]methanone.

4. The combination of claim 1, wherein the compound of formula (I) is
(S)-1-((2,3-dihydrobenzo[b][1,4]dioxin-2-yl)methyl)-4-(3-([¹¹C]-methoxymethyl)pyridin-2-yl)piperazine
and wherein the compound of formula (II) is selected from the group consisting of:
(4-{[2-(4-Chlorophenyl)imidazo[1,2-a]pyridin-3-yl]methyl}piperazin-1-yl)(6-methoxypyridin-2-yl)methanone and
(3-Chloro-6-methoxypyridin-2-yl)(3-{[2-(4-isopropylphenyl)imidazo[1,2-a]pyrimidin-3-yl]methyl}-3,8-diazabicyclo[3.2.1]oct-8-yl)methanone.

5. The combination of claim 1, wherein the compound of formula (I) is
(S)-1-((2,3-dihydrobenzo[b][1,4]dioxin-2-yl)methyl)-4-(3-([¹¹C]methoxymethyl)pyridin-2-yl)piperazine
and wherein the compound of formula (II) is
(4-{[2-(4-Chlorophenyl)imidazo[1,2-a]pyridin-3-yl]methyl}piperazin-1-yl)(6-methoxypyridin-2-yl)methanone.

6. The combination of claim 1, wherein wherein the compound of formula (I) is
(S)-1-((2,3-dihydrobenzo[b][1,4]dioxin-2-yl)methyl)-4-(3-([¹¹C]-methoxymethyl)pyridin-2-yl)piperazine
and wherein the compound of formula (II) is
(3-Chloro-6-methoxypyridin-2-yl)(3-{[2-(4-isopropylphenyl)imidazo[1,2-a]pyrimidin-3-yl]methyl}-3,8-diazabicyclo[3.2.1]oct-8-yl)methanone.

7-8. (canceled)

9. A pharmaceutical composition comprising the combination of claim 1 and one or more inert, nontoxic, pharmaceutically suitable excipients.

10. A pharmaceutical composition comprising the combination of claim 1 and one or more further active ingredients selected from the group consisting of muscarinic receptor antagonists, mineralocorticoid receptor antagonists, diuretics, and corticosteroids.

11. (canceled)

12. A method of treating and/or preventing respiratory disorders, sleep-related respiratory disorders, obstructive sleep apnoea, central sleep apnoea, snoring, cardiac arrhythmias, neurodegenerative disorders, neuroinflammatory disorders and neuroimmunological disorders in humans and animals by administration of an effective amount of the combination of claim 1 to a subject in need thereof.

13. (canceled)

14. A method of treating and/or preventing respiratory disorders, sleep-related respiratory disorders, obstructive sleep apnoea, central sleep apnoea, snoring, cardiac arrhythmias, neurodegenerative disorders, neuroinflammatory disorders and neuroimmunological disorders in humans and animals by administration of an effective amount of the composition of claim 9 to a subject in need thereof.

15. A method of treating and/or preventing respiratory disorders, sleep-related respiratory disorders, obstructive sleep apnoea, central sleep apnoea, snoring, cardiac arrhythmias, neurodegenerative disorders, neuroinflammatory disorders and neuroimmunological disorders in humans and animals by administration of an effective amount of the composition of claim 10 to a subject in need thereof.
